(19) United States
(12) Patent Application Publication
(10) Pub. No.: US 2025/0155437 A1
(43) Pub. Date: May 15, 2025

(54) METHODS AND SYSTEMS FOR DETECTING HUMAN PAPILLOMAVIRUSES (HPV) IN BIOLOGICAL SAMPLES

(71) Applicant: Northwestern University, Evanston, IL (US)

(72) Inventors: Laimonis A. Laimins, Evanston, IL (US); Conor Winslow Templeton, Evanston, IL (US)

(21) Appl. No.: 18/943,280

(22) Filed: Nov. 11, 2024

Related U.S. Application Data
(60) Provisional application No. 63/597,941, filed on Nov. 10, 2023.

(51) Int. Cl. G01N 33/569

(52) U.S. Cl. CPC G01N 33/56983; G01N 2333/025

(57) ABSTRACT

Provided herein are methods and systems for detecting one or more human papillomaviruses (HPV) in a biological sample. The methods can include detecting binding of an antibody or antibody fragment to DNA-RNA hybrids.

### CROSS-REFERENCE TO RELATED APPLICATIONS

This application claims priority to U.S. Provisional Application No. 63/597,941 filed on Nov. 10, 2023. The contents of which are herein incorporated by reference in its entirety.

### STATEMENT REGARDING FEDERALLY SPONSORED RESEARCH OR DEVELOPMENT

This invention was made with government support under grant numbers CA142861 and CA059655 awarded by the National Institutes of Health. The government has certain rights in the invention.

### REFERENCE TO A SEQUENCE LISTING

The contents of the electronic sequence listing (702581_02580_SL_ST26.xml; Size: 39,723 bytes; and Date of Creation: Nov. 8, 2024) is herein incorporated by reference in its entirety.

### BACKGROUND

R-loops are trimeric structures consisting of an RNA-DNA hybrid and a displaced DNA strand that are formed during transcription. These structures form at promoters and sites of termination to regulate transcription; however, aberrant R-loop formation or turnover can lead to genomic instability and DNA breaks. R-loop homeostasis is maintained by enzymes such as RNase H1 and H2 as well as senataxin. RNase H1 can degrade the RNA moiety in R-loops and controls the formation of aberrant R-loops. Similarly, senataxin resolves R-loops that form at the 3′ end of the transcribed genes. In normal cells, R-loops have functions in regulating transcription, DNA damage repair, and other functions, while in cancers, they can result in genomic instability.

### SUMMARY

In one aspect, a method for detecting one or more human papillomaviruses (HPV) in a biological sample is provided. The method can include: exposing a biological sample to an antibody or antibody fragment that binds to DNA-RNA hybrids; detecting binding of the antibody or antibody fragment to DNA-RNA hybrids in the biological sample; and determining that the biological sample contains HPV based on the detecting binding of the antibody or antibody fragment to DNA-RNA hybrids in the biological sample.

In another aspect, a method for detecting one or more human papillomaviruses (HPV) in a biological sample is provided. The method can include: exposing a first portion of the biological sample to an antibody or antibody fragment that binds to p16 protein; detecting binding of the antibody or antibody fragment to p16 in the first portion of the biological sample; exposing a second portion of the biological sample to an antibody or antibody fragment that binds to DNA-RNA hybrids; and detecting binding of the antibody or antibody fragment to DNA-RNA hybrids in the second portion of the biological sample.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-1H. R-loop levels are increased in HPV positive cells. (FIG. 1A) Dot-blot analysis of nucleic acid extracts from undifferentiated (UD) HFKs, HFK 31, and CIN 612 cells or differentiated (D) for 72 hr in a high calcium medium probed with S9.6 antibody showing increased levels in HPV positive cells. Left panel shows dot-blot assays using technical triplicates. Right panel shows quantitation of data from 4 experiments and is plotted as the average of the mean with the error bars representing the standard error of the mean (SEM; A, right; ns, not significant; ****, p<0.0001). (FIG. 1B) Graph showing quantitation of dot-blot assays of nucleic acids isolated from undifferentiated HFKs, CIN 612, HFK 31, HFK 16, and HFK 18 cells probed with the S9.6 antibody. Quantification of three biological replicates is plotted as the average and SEM (B; *, p<0.05; ***, p<0.001; ****, p<0.0001). (FIG. 1C) R-loops form as puncta within the nuclei of HPV positive cells. Immunofluorescence analysis of undifferentiated HFKs, HFK 31, and CIN 612 cells using the S9.6 antibody (n=3, a representative field is shown). (FIG. 1D) Hematoxylin and eosin staining was performed on paraffin-embedded tissue from high-grade cervical carcinomas that included normal tissue at adjacent margins. Cross sections of the same tissue were used for immunofluorescence analysis with antibodies recognizing R-loops (S9.6) and (FIG. 1E) DAPI. (FIG. 1F) DNA:RNA immunoprecipitation assays (DRIP) of HFKs, HFK 31, and CIN 612 cells were performed, and immunoprecipitated chromatin was quantified by qPCR analysis. R-loops form on the viral genome and ALU sequences in HPV positive cells. Fold enrichment for each primer set over IgG is shown: (S9.6ₓ/IgGₓ)/(S9.6_{HF}K/IgG_{HFK}), where x is Ct values from either HFK 31 or CIN 612. The error bars represent the SEM of six biological replicates. (FIG. 1G) DRIP assays for R-loops were performed and analyzed by qPCR for BRCA1 ORF, E7 ORF, or URR sequences in HFKs, HFK 31, or CIN 612 cells. Three biological replicates were analyzed (ns, not significant; *, p<0.05; **, p<0.01; ***, p<0.001; ****, p<0.0001). (FIG. 1H) DRIP assays using HFK 31, and CIN 612 cells were performed, and immunoprecipitated nucleic acid signals were quantified by qPCR analysis using primers from the p97 promoter, E2, early poly-A, and late poly-A regions of HPV 31. Fold enrichment for each primer set was measured as (S9.6ₓ/IgGₓ).

FIG. 2. S9.6 specificity for R-loop detection through dot blot analysis. The S9.6 antibody is specific for R-loops in HPV positive cells. Nucleic acids were extracted from HFK 31 and CIN 612 cells and either left untreated or treated with RNase H prior to S9.6 antibody dot blot analysis. Increasing DNA concentrations were loaded, ranging from 25 to 800 ng. A subset was denatured and probed with a ssDNA antibody to control for DNA loading, while the rest were probed with the S9.6 antibody. A representative image of three biological replicates is shown.

FIG. 3. S9.6 staining of fixed tissues is sensitive to RNase H treatment. Immunofluorescence analysis was performed on paraffin-embedded tissue from high-grade cervical carcinomas, including normal tissue at adjacent margins. Immunohistochemistry identified normal tissue and tumor. Tissues were permeabilized and either left untreated (top), digested with 2.5 U of RNase T and III (middle), or digested with 2.5 U RNase H (bottom). Cross sections of the same tissue were used for immunofluorescence analysis with antibodies recognizing R-loops (S9.6) and DAPI.

FIGS. 4A-4F. RNase H1 levels and localization are altered in HPV positive cells. Levels of R-loop resolving enzymes are increased in undifferentiated HPV positive cells. (FIG. 4A) Western blot analysis for RNase H1 for HFK, HFK 31, and CIN 612 cells either undifferentiated (UD) or differentiated (D) for 72 hr in a high calcium medium (n=3, a representative image is shown). Senataxin is post-translationally modified and its modified forms are marked by asterisks [65, 66]. Non-specific bands are seen in other studies [67]. (FIG. 4B, FIG. 4C) RNase H1 is recruited to nuclear foci and nucleoli in HPV positive cells. Immunofluorescence analysis of RNase H1 and γH2AX or RNase H1 and nucleolin in undifferentiated HFK, HFK 31, and CIN 612 cells (n=3; a representative field is shown). Quantification of nucleolin foci in HFKs or CIN 612 cells is shown (C, right) (n=3, 150 cells) (FIG. 4D). RNase H1 is bound to both viral and cellular sequences in HPV positive cells. Graph showing chromatin immunoprecipitation analysis of RNase H1 binding to ALU or HPV 31 URR sequences using extracts from HFKs, HFK 31, and CIN 612 cells. Fold enrichment is quantified as (RNase H1ₓ/IgGₓ)/(RNase H1_{HFK}/IgG_{HFK}) where x are Ct values from either HFK 31 or CIN 612 cells (n=3; ns, not significant; **, p<0.01; ****, p<0.0001). (FIG. 4E). Depletion of RNase H1 increases R-loop levels and impairs the maintenance of HPV genomes in undifferentiated CIN 612 cells. Cells were infected with retroviruses expressing either scramble control or 3 different shRNAs against RNase H1. Left panel shows western blot analysis of CIN 612 cells transduced with shRNA sequences targeting RNase H1 together with SETX, Mre11, γH2AX or GAPDH loading control. The right panel shows S9.6 dot blots performed, showing that depletion of RNase H1 resulted in increased global R-loop levels. The data is plotted as the average of three biological replicates and the error bars are the SEM (****, p<0.0001). (FIG. 4F). RNase H1 depletion reduces HPV episomes within CIN 612 cells. Southern blot analysis showing cells stably expressing a scrambled control or shRNAs to RNase H1 depleted. A representative Southern blot is shown (left), and the panel on the right shows the genome copy numbers quantified as the average and SEM of three biological replicates (right; ****, p<0.0001).

FIG. 5. Senataxin is not recruited to nucleoli within HPV positive cells. Immunofluorescence analysis of undifferentiated HFKs, HFK31, and CIN 612 cells using the senataxin and nucleolin antibody (n=3, a representative field is shown).

FIGS. 6A-6D. Depletion of RNase H1 alters viral and cellular gene expression in HPV positive cells. (FIG. 6A) RT-qPCR was performed for viral transcripts encoding either E6, E7, or E1 from CIN 612 cells stably expressed scramble control shRNA (Scrm) or CIN 612 cells stably expressed shRNAs to RNase H1 (clone 3-1 or 5-1). The data is plotted as the average of three biological replicates and the error bars are the SEM (**, p<0.01). (FIG. 6B) RNA-seq analysis identifying major pathways whose expression is altered relative in cells with shRNAs to RNase H1 relative to scramble control CIN 612 cells. The graph represents the results of gene ontology enrichment analysis of the RNA-seq data. The number of genes in each pathway that exhibit decreased transcript levels comparing scrm control to shRNase H1 CIN 612 cells and p-value are plotted on the y-axis at the right (p<2.03×10⁻⁴³ to p<5.61×10⁻¹⁹). mRNA-sequencing analysis was performed in biological duplicate on each cell line: scrm CIN 612, shRNH1 3-1 CIN 612, and shRNH1 5-1 CIN 612. (FIG. 6C) Western blot analysis of representative genes downregulated at the transcript level through RNA-seq. Levels of FANCD2, Mre11, RNase H1, and ATR levels decreased upon depletion of RNase H1 consistent with the RNA-seq data (n=4, a representative image is shown). (FIG. 6D) Graph identifying pathways where expression of genes was increased in shRNase H1 CIN 612 cells relative to scramble control as determined by gene ontology enrichment analysis of RNA sequencing data (n=4). P-values are shown on the y-axis to the right (p<1.18×10⁻⁵ to p<0.0021).

FIG. 7. Overexpression of RNase H1 reduces R-loop levels within CIN 612 cells. Overexpression of RNase H1 substantially reduced R-loop levels within HPV positive cells. Dot blot analysis of Scrm, o/eRNH1, and parental CIN 612 cells to HFKs using the S9.6 antibody (n=4; a representative image is shown). The data is plotted as the average of four replicated with the error bars representing SEM.

FIGS. 8A-8E. RNase H1 overexpression impairs genome maintenance and promotes the expression of immune response genes. (FIG. 8A) Overexpression of RNase H1 reduced HPV episomes. A Southern blot is shown of scrm CIN 612 cells compared to CIN 612 cells either overexpressing RNase H1 (o/e RNH1) or depleted of RNase H1. The associated graph shows data from the average of four biological replicates, and the error bars are the SEM (***, p<0.001). (FIG. 8B) Viral transcription is hindered by overexpression of RNase H1. RT-qPCR analysis of scrm or o/eRNH1 CIN 612 cells for viral transcripts encoding E1, E6, and E7 (n=3; ****, p<0.0001). (FIG. 8C) RNA-seq analysis of cells overexpressing RNase H1 (o/e CIN612) showing pathways whose expression was increased as identified by gene ontology enrichment analysis. P-values are shown on the y-axis to the right (p<2.72×10⁻²⁷ to p<0.0063) (FIG. 8D) Western blot analysis corresponding to representative genes upregulated at the transcript level through RNA-seq (n=4, a representative blot is shown). Rig-I, TRIM25, DDX1, and RPA2 levels are shown to be increased. (FIG. 8E) R-loops are responsible for ˜50% of the DNA breaks within CIN 612 cells. COMET assays were performed on HFKs and CIN 612 cells either transduced with a scrambled shRNA sequence, depleted of RNase H1 (shRNH1), or overexpressing RNase H1 (o/eRNH1). Tail moment (Tail length (px)×Tail DNA %) was calculated from three independent experiments. Individual points are plotted with the bars representing a 95% confidence interval from the mean (n=50; a representative graph is shown).

FIGS. 9A-9D. HPV 31 E6 is the primary viral factor responsible for increased levels of R-loops. (FIG. 9A). Keratinocytes stably infected with retroviruses expressing HPV 31 E6 or E7 were examined for R-loop levels by S9.6 dot blot analysis. Undifferentiated (UD) or differentiated (D) HFKs infected with empty vector control, HFK E6, or HFK E7 cells were examined (n=3; ns, not significant; ****, p<0.0001). (FIG. 9B) Western blot analysis for R-loop regulatory factors SetX, Mre11, RNase H1, and γH2AX in undifferentiated HFKs, HFK E6, or HFK E7 cells are shown (n=3; a representative image is shown). (FIG. 9C). E6 expression induces nuclear R-loop puncta in HFK E6 expressing cells similar to those seen in HPV positive cells. Immunofluorescence analysis for S9.6 antibody and DAPI is shown (n=3; a representative field is shown). (FIG. 9D) DRIP analysis of HFKs, HFK E6, or HFK E7 analyzing R-loop levels on ALU respective elements or BRCA1 coding sequences is shown for HFKs, HFK E6, and HFK E7 (n=3; ****, p<0.0001).

FIGS. 10A-10C. HPV 31 E6 alters RNase H1 subcellular localization. RNase H1 but not senataxin exhibits an altered subcellular localization in HFK #6 cells. (FIG. 10A) Immunofluorescence analysis of RNase H1 and γH2AX or RNase H1 and nucleolin in undifferentiated HFK, HFK E6, and HFK E7 cells (n=3; a representative field is shown). (FIG. 10B) Immunofluorescence analysis of undifferentiated HFKs, HEF E6, and HFK E7 cells using antibodies to senataxin and nucleolin antibody (n=3; a representative field is shown). (FIG. 10C). Chromatin immunoprecipitations of sequences bound by RNase H1 in HFKs, HFK E6, and HFK E7. Fold enrichment was calculated as RNase H1ₓ/IgGₓ)/(RNase H1_{HFK}/IgG_{HFK}) where x is the Ct value from the HFK E6 or HFK E7 cells. The data is plotted as the average of three biological replicates, and the error bars represent the SEM (****, p<0.0001).

FIGS. 11A-11D. p53 inactivation or depletion drives R-loop formation within HFKs expressing HPV 31 E7. Transient depletion of p53 with transfected siRNAs in HFK E7 cells increases R-loop levels. (FIG. 11A) HFKs expressing E6 or E7 either not transfected (−), transfected with an siCntrl vector (C), or transfected with siRNA targeting p53. Samples were collected 24 to 72 hr post-transfection and silencing of p53 was validated by western blot analysis. (FIG. 11B) S9.6 dot blot analysis of the same experimental samples as described above (*, p<0.05; ****, p<0.0001). Assays were repeated 4 times with similar results. Inhibition of p53 using pifithrin a similarly increases R-loop levels within E7 expressing HFKs (FIG. 11C, FIG. 11D). Western blot analysis of HFK, HFK E6, and HFK E7 cells treated with pifithrin a for 24 hr (a representative image is shown, n=3). (FIG. 11D) S9.6 dot blot analysis of the same experimental samples as described above in C (n=3; error bars represent the SEM). Ns, not significant; ****, p<0.0001.

FIGS. 12A-12B. Comparison of p16 and R-loop staining of normal and HPV positive tumor tissue. FIG. 12B is a close up view of the yellow inset in FIG. 12A.

FIGS. 13A-13H. Distribution of R-loops in HPV positive cells compared to normal keratinocytes. (FIG. 13A) S9.6 dot blot analysis from whole cell nucleic acid extracts of normal keratinocytes (HFKs), transfected, HPV positive keratinocytes (HFK-31), and HPV positive keratinocytes derived from a cervical CIN 1 lesion (CIN 612). Total nucleic acid levels were measured via methylene blue staining (top), and the specificity of the S9.6 monoclonal antibody was assessed through RNase H treatment. (FIG. 13B) DNA:RNA immunoprecipitation assays (DRIP) were performed on HFKs, HFK 31, and CIN 612 cells for six representative cellular sites, and immunoprecipitated chromatin was analyzed by quantitative PCR (qPCR). Primers mapped to EGR1, RPL13a, SLC35B2, and LGALS2 were used as positive controls for regions previously characterized to contain R-loops; SNRPN was used as a negative control, while MYADM has variable reports of its association with R-loops [12, 16, 35-37]. Fold enrichment for each primer set over HFKS9.6 is plotted: (S9.6ₓ/IgGₓ)/(S9.6_{HFK}/IgG_{HFK}) where x is Ct values from either HFK 31 or CIN 612 cells. The error bars represent the standard error of the mean (n=3, ns, not significant; p<0.05, *; p<0.001, ***; p<0.0001, ****). (FIG. 13C) DRIP-qPCR of three regions on the HPV 31 genome in comparison to ALU repetitive cellular elements. DRIP-qPCR was performed on HFKs, HFK 31, and CIN 612 cells using primers mapping to ALU elements and viral genomic elements (early polyA site, upstream regulatory region (URR), and the late polyA site). Percentage input was plotted: Input %=100/2^{(_}^{^{−}}^{Ct [nonmalized to input control])} The error bars represent the standard error of the mean (n=3, p<0.001, ***; p<0.00001, ****). (FIG. 13D) Metaplot distribution of S9.6 signal (IP−input) through genic regions, including 2 kb flanking upstream or downstream (n=2, top). Heat map of S9.6 intensity through genic and 2 kb flanking regions (n=2, bottom). Depth graphs of input normalized S9.6 reads through MYADM (FIG. 13F), LGALS2 (FIG. 13G), RPL13a (FIG. 13E), and ZNF554 (FIG. 13H) in normal keratinocytes (black) and CIN 612 cells (red).

FIGS. 14A-14E. R-loops form preferentially on genes in pathways responsible for the cancer progression and viral pathogenesis in CIN 612 cells. (FIG. 14A) Venn diagram of the genomic regions containing R-loop peaks (MACS) overlapping between normal keratinocytes and HPV positive cells (CIN612) (n=2, a representative image is shown). Total R-loops in CIN 612 (pink) and HFK (red) with common sites (brown). (FIG. 14B) MA plot analysis of the common R-loop containing genes between CIN 612 cells and normal keratinocytes. Log₂ enrichment of R-loop levels over the matched input control is plotted on the x-axis, and Log₂ enrichment of R-loop levels in genes present within CIN 612 cells over HFKs is plotted on the y-axis. (FIG. 14C) Distribution of R-loop peaks relative to genomic locations in HFKs and CIN 612 cells. CHIPSEEKER was used to analyze the location of R-loop reads within each sample. HOMER was used to identify the location of where R-loop peaks occurred within HFK (FIG. 14E) and CIN 612 (FIG. 14D) cells. Intergenic R-loops were filtered out, leaving R-loops that fell within introns, exons, TES, TSS, 3′UTR, and 5′UTR. Common genes found in both HFKs and CIN 612 cells were also filtered out. Pathway analysis was then performed on the genes to which these R-loops were assigned to either the CIN 612 cells or the HFKs using Shiny GO 0.80. KEGG pathways or molecular function analyses are shown.

FIGS. 15A-15H. Formation of R-loops at unique sites in HPV positive, CIN 612 cells correlates with differential gene expression. (FIG. 15A) Venn diagram of the differentially expressed genes in precancerous CIN 612 cells compared to normal keratinocytes (n=2, RPKM>0) Genes upregulated in CIN 612 (green), upregulated in HFKs (red), and those with no difference (<1 Log₂ FC, brown). (FIG. 15B) MA plot analysis of the differentially expressed genes exhibiting a similar distribution of downregulation and upregulation. The differentially expressed genes were divided between those upregulated (FIG. 15C) and downregulated (FIG. 15D) in CIN 612 cells compared to HFKs. Pathway analysis of the biological processes of these genes was performed using Shiny GO 0.80. (FIG. 15E) mRNA levels of genes R-loop positive or negative in HFKs and CIN 612 cells. The line represents the mean (p<0.0001; ****). (FIG. 15F) Around 25% of all differentially expressed genes are associated with R-loops only in precancerous CIN 612 cells. R-loop peaks in the genic or 2 kb flanking regions of genes in HFKs or those that were common to both HFKs and CIN 612 cells were filtered out. The remaining genes were screened against the differentially expressed genes in CIN 612 cells compared to HFKs. Fold enrichment of S9.6 reads over input is plotted on the X-axis, while fold change of mRNA levels in CIN 612 cells compared to HFKs is plotted on the Y-axis (left). Pathway analysis of the R-loop containing genes upregulated (FIG. 15G) and downregulated (FIG. 15H) in CIN 612 cells.

FIGS. 16A-16B. Reduction of R-loops through RNase H1 overexpression in CIN 612 cells identifies genes that are functionally dependent on their formation. (FIG. 16A) Major pathways whose expression is dependent on R-loops present only in CIN 612 cells as determined by RNase H1 overexpression. Pathway analysis was performed using the Hallmark database in Shiny GO 0.80 of R-loop regulated genes that contain R-loops unique to the CIN 612 cells. (FIG. 16B) Innate immune response genes whose expression is regulated by R-loops that are present only in CIN 612 cells. Fold changes are normalized to the mRNA counts in normal keratinocytes (n=2). Linkages to H3K36me3 and γH2AX histones are shown on the right.

FIGS. 17A-17B. Modified histones are differentially associated with actively transcribed genes and R-loops in HPV positive cells. (FIG. 17A) Total mRNA levels of genes that are H3K36me3 positive or negative (left) as well as H3K9me3 positive or negative (right) in HFKs (top panels) and CIN 612 cells (bottom two panels). The red line represents the mean. The error bars are SEM (ns, not significant; p<0.0001, ****). (FIG. 17B) Venn diagrams of the genomic regions containing H3K36me3 (left) or H3K9me3 (right) and R-loop peaks (MACS) overlapping between the HPV negative (HFKs) and positive (CIN612) cells (n=2, a representative image is shown). Genes containing H3K36me3 or H3K9me3 peaks (red), R-loop peaks (green), and both (brown). P-values represent that the overlap between genic marks is above or below that expected from random distribution. RF represents representation factors. RF values greater than 1 suggest more overlap than expected from random distribution, while RF values less than 1 imply less overlap.

FIGS. 18A-18E. γH2AX is associated with R-loop formation and H3K36me3 deposition in CIN 612 cells. (FIG. 18A) mRNA levels of genes γH2AX that are negative or positive in HFKs (left) and CIN 612 (right) cells. The red line represents the mean. The error bars are SEM (p<0.05, *; p<0.0001, ****). (FIG. 18B) Venn diagrams of the genes containing γH2AX and R-loop peaks (MACS) overlapping between the HFKs (left) and CIN 612 (right) cells (n=2). Genes containing γH2AX peaks (red), R-loop peaks (green), and both (brown). P-values represent that the overlap between genic marks is above or below that expected from random distribution. RF represents representation factors. RF values greater than 1 suggest more overlap than expected from random distribution, while RF values less than 1 imply less overlap. Venn diagrams of the genes containing γH2AX, H3K36me3, and R-loops in CIN 612 cells (FIG. 18C) and the GO biological processes pathway analysis of these genes (FIG. 18D). (FIG. 18E) A table showing DNA repair and metabolism genes that are differentially expressed in CIN 612 cells with corresponding marks of R-loops, γH2AX, and H3K36me3, which are present on these genes only in the precancerous cells.

FIGS. 19A-19C. Input normalized S9.6 reads of two regions associated with R-loops in HFK and CIN 612 cells. Depth graphs of S9.6 reads normalized to the corresponding cell line's input control reads. Reads were binned into 300 bp regions during quantification using deeptools2 (BAMcompare). Two regions are pictured: Lig4 (FIG. 19A) and CALML5 (FIG. 19B). The red represents CIN 612 cells, while the black represents HFKs (n=2, mean is shown).

FIGS. 20A-20B. R-loop association with RNA at different genomic locations does not demonstrate any preferential increases or decreases in either HFKs or CIN 612 cells. (FIG. 20A) Fold enrichment of S9.6 reads over input was taken from two independent DRIP-sequencing experiments. (FIG. 20B) Genes that contained R-loops in the coding sequence or the corresponding 2 kb flanking regions were then analyzed for their mRNA levels from two independent RNA sequencing experiments. These genes were plotted as average mRNA read counts (y-axis) versus fold enrichment of R-loops over input (x-axis). The line of best fit was calculated using Least Squares Regression (GraphPad Prism), where dashed lines represent the 95% confidence intervals (Q=1% for detection of outliers, red). The equation for the line of best fit is shown for each graph in the top right corner.

FIGS. 21A-21B. Pathway analysis of differentially expressed genes in CIN 612 cells overexpressing (o/e) RNase H1 using the Hallmark MSigDB database. (FIG. 21A) Cumulative numbers of differentially expressed genes in the CIN 612 cells overexpressing RNase H1 versus parental CIN 612 cells (red=downregulated, green=upregulated). The corresponding pathway analysis used Shiny GO 0.80 (http://bioinformatics.sdstate.edu/go/) and the Hallmark MSigDB database (downregulated=left, upregulated=right). Pathways of particular interest were those upregulated upon overexpression of RNase H1, including those responsible for an interferon alpha/gamma response, IL6 JAK STAT3 signaling, and IL2 STAT5 signaling (right). (FIG. 21B) Flow chart of how R-loop dependent gene expression was determined in CIN 612 cells. Negatively regulated genes were identified as those with reduced expression in parental CIN 612 cells compared to normal keratinocytes, which then increased in expression upon loss of R-loops through RNase H1 overexpression (left). 542 genes were identified, most of which were involved in immune surveillance and signaling. Positively regulated genes were identified as those with increased expression in parental CIN 612 cells compared to normal keratinocytes, which then decreased in expression upon loss of R-loops upon RNase H1 overexpression (right). 722 genes were identified, many of which were involved in DNA metabolism. Of the 1,264 genes identified as being R-loop regulated in CIN 612 cells, 833 of them contained R-loops only in the CIN 612 cells. These genes were deemed as being functionally regulated by R-loop formation for the analyses performed in FIG. 16A.

FIGS. 22A-22F. H3K36me3 and H3K9me3 are differentially present on host chromatin within CIN 612 cells compared to HFKs. (FIG. 22A) Venn diagram of the genomic regions containing H3K36me3 peaks (left) and H3K9me3 peaks (right) (MACS) overlapping between the HPV negative (HFKs) and positive (CIN612) cells (n=2, a representative image is shown). (FIG. 22B) Fingerprint plot of H3K36me3 and H3K9me3 distribution compared to input control in HFKs and CIN 612 cells. (FIGS. 22C-D) Distribution of H3K36me3 and H3K9me3 reads relative to genomic locations in HFKs (FIG. 22D) and CIN 612 (FIG. 22C) cells. CHIPSEEKER was used to analyze the location of each modified histones' reads within each sample (FIG. 22C, left and FIG. 22D, right). Deeptools2 (BAMcompare) was used to input normalize H3K36me3 (FIG. 22C, top right and FIG. 22D, top left) and H3K9me3 (FIG. 22C, bottom right and FIG. 22D, bottom left). Regions analyzed were set as 500 bp, flanking the coding sequence, and the average genic profile was visualized (ComputeMatrix). HOMER was used to identify the location of where H3K36me3 (FIG. 22E) or H3K9me3 (FIG. 22F) peaks occurred within HFK and CIN 612 cells. Intergenic histone marks were filtered out, leaving only histone marks that fell within introns, exons, TES, TSS, 3′UTR, and 5′UTR. Common genes found in both HFKs and CIN 612 cells were also filtered out. Pathway analysis was then performed on the genes to which these histone marks were assigned in the CIN 612 cells or the HFKs using Shiny GO 0.80. The GO biological process database was used for all analyses.

FIGS. 23A-23D. Pathway analyses of genes enriched with modified histones (γH2AX, H3K36me3, or H3K9me3) and R-loops in CIN 612 cells. Genes containing a modified histone mark and R-loops unique to CIN 612 cells (FIGS. 23A-C) or HFKs (FIG. 23D) were analyzed using Shiny GO 0.80 and the GO biological process database. Due to the lack of H3K9me3 or γH2AX and R-loop containing genes in HFKs, no analysis of those genes is depicted.

FIGS. 24A-24E. γH2AX is significantly enriched on host chromatin and on genes responsible for important processes during HPV infection in CIN 612 cells. (FIG. 24A) Distribution of γH2AX reads relative to genomic locations in HFKs and CIN 612 cells. CHIPSEEKER was used to analyze the location of R-loop reads within each sample (left and right). (FIG. 24B) Fingerprint plot of γH2AX distribution compared to input control in HFKs and CIN 612 cells. (FIG. 24C) Deeptools2 (BAMcompare) was used to input normalize γH2AX reads. Regions analyzed were set as 500 bp, flanking the coding sequence, and the average genic profile was visualized (ComputeMatrix). (FIG. 24D) Venn diagram of the genomic regions containing γH2AX peaks (right) (MACS) overlapping between HFK and CIN 612 cells (n=2, a representative image is shown). (FIG. 24E) HOMER was used to identify the location of where γH2AX peaks occurred within HFK (bottom) and CIN 612 cells (top). Intergenic histone marks were filtered out, leaving only histone marks that fell within introns, exons, TES, TSS, 3′UTR, and 5′UTR. Common genes found in both HFKs and CIN 612 cells were also filtered out. Pathway analysis was then performed on the genes to which these histone marks were assigned in the CIN 612 cells or the HFKs using Shiny GO 0.80. The GO biological process database was used for all analyses.

FIGS. 25A-25D. Validation of DRIP-sequencing replicates. (FIG. 25A) XY correlation plots of S9.6 reads in biological replicates from HFKs (top left) and CIN 612 cells (top right). Pearson's coefficient is labeled on each respective scatter plot. XY correlation plot comparing HFK S9.6 reads to CIN 612 S9.6 reads with Spearman's coefficient labeled (bottom middle). These data support that there is a strong agreement between the S9.6 pulldown replicates in HFK and CIN 612 cells and that there are higher read counts in similar genomic regions in the CIN 612 cells. (FIG. 25B) Heatmap of Pearson coefficient values between input controls and S9.6 pulldown assays in HFK and CIN 612 cells. A strong correlation is seen among the S9.6 pulldown assays, suggesting that a majority of the reads are located in similar genomic regions. (FIG. 25C) Fingerprint plot analysis of input control samples and S9.6 pulldown replicates in HFK and CIN 612 cells. Input DNA reads are broadly distributed across the genome, while S9.6 reads are enriched on a much smaller proportion of DNA. (FIG. 25D) Principal component analysis of input control samples and S9.6 pulldown replicates in HFK and CIN 612 cells. A high degree of clustering is seen between the S9.6 replicates from the HFK and CIN 612 cells.

FIGS. 26A-26F. Validation of Modified Histones ChIP-sequencing replicates. (FIG. 26A) Heatmap of Pearson coefficient values between input controls and H3K36me3 pulldown assays in HFK and CIN 612 cells. (FIG. 26B) Principal component analysis of input control samples and H3K36me3 pulldown replicates in HFK and CIN 612 cells. (FIG. 26C) Heatmap of Pearson coefficient values between input controls and H3K9me3 pulldown assays in HFK and CIN 612 cells. (FIG. 26D) Principal component analysis of input control samples and H3K9me3 pulldown replicates in HFK and CIN 612 cells. (FIG. 26E) Heatmap of Pearson coefficient values between input controls and γH2AX pulldown assays in HFK and CIN 612 cells. (FIG. 26F) Principal component analysis of input control samples and γH2AX pulldown replicates in HFK and CIN 612 cells.

### DETAILED DESCRIPTION

High-risk human papillomaviruses (HPV) are the etiological agents of genital and oropharyngeal cancers. Although prophylactic vaccines are effective in blocking initial infection by these viruses, they are not effective against existing lesions. Understanding the mechanisms regulating HPV pathogenesis is therefore important for the identification of new biomarkers and for the development of novel therapeutics. As described herein, high levels of trimeric RNA:DNA structures called R-loops are present in HPV positive cells derived from low-grade cervical lesions as well as in squamous cell carcinomas. These elevated R-loop levels play a role in both viral gene expression and DNA replication. R-loops play a role in cellular regulators of HPV pathogenesis, and are useful as novel biomarkers for viral infection or therapeutic targets.

In various aspects, as described herein, methods are provided that include detecting binding of an antibody or antibody fragment to RNA-DNA hybrids in a biological sample. The methods can further include determining that the biological sample contains HPV based on the binding of an antibody or antibody fragment to RNA-DNA hybrids. In various aspects, such a method exhibits increased sensitivity to identifying HPV positive lesions as compared to the current conventional method, which relies on the presence of the p16 protein in a biological sample. In various aspects, such increased sensitivity can allow for increased identification of HPV positive tissue, which can lead to better treatments, such as identifying the margins of a tumor for potential removal.

### Definitions

The disclosed subject matter may be further described using definitions and terminology as follows. The definitions and terminology used herein are for the purpose of describing particular embodiments only and are not intended to be limiting.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

### Polynucleotides and Synthesis Methods

The terms “nucleic acid” and “oligonucleotide,” as used herein, refer to polydeoxyribonucleotides (containing 2-deoxy-D-ribose), polyribonucleotides (containing D-ribose), and to any other type of polynucleotide that is an N glycoside of a purine or pyrimidine base. There is no intended distinction in length between the terms “nucleic acid”, “oligonucleotide” and “polynucleotide”, and these terms will be used interchangeably. These terms refer only to the primary structure of the molecule. Thus, these terms include double- and single-stranded DNA, as well as double- and single-stranded RNA. For use in the present methods, an oligonucleotide also can comprise nucleotide analogs in which the base, sugar, or phosphate backbone is modified as well as non-purine or non-pyrimidine nucleotide analogs.

Oligonucleotides can be prepared by any suitable method, including direct chemical synthesis by a method such as the phosphotriester method of Narang et al., 1979, Meth. Enzymol. 68:90-99; the phosphodiester method of Brown et al., 1979, Meth. Enzymol. 68:109-151; the diethylphosphoramidite method of Beaucage et al., 1981, Tetrahedron Letters 22:1859-1862; and the solid support method of U.S. Pat. No. 4,458,066, each incorporated herein by reference. A review of synthesis methods of conjugates of oligonucleotides and modified nucleotides is provided in Goodchild, 1990, Bioconjugate Chemistry 1(3): 165-187, incorporated herein by reference.

The term “amplification reaction” refers to any chemical reaction, including an enzymatic reaction, which results in increased copies of a template nucleic acid sequence or results in transcription of a template nucleic acid. Amplification reactions include reverse transcription, the polymerase chain reaction (PCR), including Real Time PCR (see U.S. Pat. Nos. 4,683,195 and 4,683,202; PCR Protocols: A Guide to Methods and Applications (Innis et al., eds, 1990)), and the ligase chain reaction (LCR) (see Barany et al., U.S. Pat. No. 5,494,810). Exemplary “amplification reactions conditions” or “amplification conditions” typically comprise either two or three step cycles. Two-step cycles have a high temperature denaturation step followed by a hybridization/elongation (or ligation) step. Three step cycles comprise a denaturation step followed by a hybridization step followed by a separate elongation step.

The terms “target,” “target sequence”, “target region”, and “target nucleic acid,” as used herein, are synonymous and refer to a region or sequence of a nucleic acid which is to be amplified, sequenced, or detected.

The term “hybridization,” as used herein, refers to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which hybridization of fully complementary nucleic acid strands is strongly preferred are referred to as “stringent hybridization conditions” or “sequence-specific hybridization conditions”. Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art (see, e.g., Sambrook et al., 1989, Molecular Cloning-A Laboratory Manual, Cold Spring Harbor Laboratory, Cold Spring Harbor, New York; Wetmur, 1991, Critical Review in Biochem. and Mol. Biol. 26(3/4):227-259; and Owczarzy et al., 2008, Biochemistry, 47: 5336-5353, which are incorporated herein by reference).

The term “primer,” as used herein, refers to an oligonucleotide capable of acting as a point of initiation of DNA synthesis under suitable conditions. Such conditions include those in which synthesis of a primer extension product complementary to a nucleic acid strand is induced in the presence of four different nucleoside triphosphates and an agent for extension (for example, a DNA polymerase or reverse transcriptase) in an appropriate buffer and at a suitable temperature.

A primer is preferably a single-stranded DNA. The appropriate length of a primer depends on the intended use of the primer but typically ranges from about 6 to about 225 nucleotides, including intermediate ranges, such as from 15 to 35 nucleotides, from 18 to 75 nucleotides and from 25 to 150 nucleotides. Short primer molecules generally require cooler temperatures to form sufficiently stable hybrid complexes with the template. A primer need not reflect the exact sequence of the template nucleic acid, but must be sufficiently complementary to hybridize with the template. The design of suitable primers for the amplification of a given target sequence is well known in the art and described in the literature cited herein.

Primers can incorporate additional features which allow for the detection or immobilization of the primer but do not alter the basic property of the primer, that of acting as a point of initiation of DNA synthesis. For example, primers may contain an additional nucleic acid sequence at the 5′ end which does not hybridize to the target nucleic acid, but which facilitates cloning or detection of the amplified product, or which enables transcription of RNA (for example, by inclusion of a promoter) or translation of protein (for example, by inclusion of a 5′-UTR, such as an Internal Ribosome Entry Site (IRES) or a 3′-UTR element, such as a poly(A)n sequence, where n is in the range from about 20 to about 200). The region of the primer that is sufficiently complementary to the template to hybridize is referred to herein as the hybridizing region.

As used herein, a primer is “specific,” for a target sequence if, when used in an amplification reaction under sufficiently stringent conditions, the primer hybridizes primarily to the target nucleic acid. Typically, a primer is specific for a target sequence if the primer-target duplex stability is greater than the stability of a duplex formed between the primer and any other sequence found in the sample. One of skill in the art will recognize that various factors, such as salt conditions as well as base composition of the primer and the location of the mismatches, will affect the specificity of the primer, and that routine experimental confirmation of the primer specificity will be needed in many cases. Hybridization conditions can be chosen under which the primer can form stable duplexes only with a target sequence. Thus, the use of target-specific primers under suitably stringent amplification conditions enables the selective amplification of those target sequences that contain the target primer binding sites.

As used herein, a “polymerase” refers to an enzyme that catalyzes the polymerization of nucleotides. “DNA polymerase” catalyzes the polymerization of deoxyribonucleotides. Known DNA polymerases include, for example, Pyrococcus furiosus (Pfu) DNA polymerase, E. coli DNA polymerase I, T7 DNA polymerase and Thermus aquaticus (Taq) DNA polymerase, among others. “RNA polymerase” catalyzes the polymerization of ribonucleotides. The foregoing examples of DNA polymerases are also known as DNA-dependent DNA polymerases. RNA-dependent DNA polymerases also fall within the scope of DNA polymerases. Reverse transcriptase, which includes viral polymerases encoded by retroviruses, is an example of an RNA-dependent DNA polymerase. Known examples of RNA polymerase (“RNAP”) include, for example, T3 RNA polymerase, T7 RNA polymerase, SP6 RNA polymerase and E. coli RNA polymerase, among others. The foregoing examples of RNA polymerases are also known as DNA-dependent RNA polymerase. The polymerase activity of any of the above enzymes can be determined by means well known in the art.

The term “promoter” refers to a cis-acting DNA sequence that directs RNA polymerase and other trans-acting transcription factors to initiate RNA transcription from the DNA template that includes the cis-acting DNA sequence.

As used herein, “expression template” refers to a nucleic acid that serves as substrate for transcribing at least one RNA that can be translated into a sequence defined biopolymer (e.g., a polypeptide or protein). Expression templates include nucleic acids composed of DNA or RNA. Suitable sources of DNA for use a nucleic acid for an expression template include genomic DNA, cDNA and RNA that can be converted into cDNA. Genomic DNA, cDNA and RNA can be from any biological source, such as a tissue sample, a biopsy, a swab, sputum, a blood sample, a fecal sample, a urine sample, a scraping, among others. The genomic DNA, cDNA and RNA can be from host cell or virus origins and from any species, including extant and extinct organisms. As used herein, “expression template” and “transcription template” have the same meaning and are used interchangeably.

In certain exemplary embodiments, vectors such as, for example, expression vectors, containing a nucleic acid encoding one or more rRNAs or reporter polypeptides and/or proteins described herein are provided. As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Such vectors are referred to herein as “expression vectors.” In general, expression vectors of utility in recombinant DNA techniques are often in the form of plasmids. In the present specification, “plasmid” and “vector” can be used interchangeably. However, the disclosed methods and compositions are intended to include such other forms of expression vectors, such as viral vectors (e.g., replication defective retroviruses, adenoviruses and adeno-associated viruses), which serve equivalent functions.

In certain exemplary embodiments, the recombinant expression vectors comprise a nucleic acid sequence (e.g., a nucleic acid sequence encoding one or more rRNAs or reporter polypeptides and/or proteins described herein) in a form suitable for expression of the nucleic acid sequence in one or more of the methods described herein, which means that the recombinant expression vectors include one or more regulatory sequences which is operatively linked to the nucleic acid sequence to be expressed. Within a recombinant expression vector, “operably linked” is intended to mean that the nucleotide sequence encoding one or more rRNAs or reporter polypeptides and/or proteins described herein is linked to the regulatory sequence(s) in a manner which allows for expression of the nucleotide sequence (e.g., in an in vitro transcription and/or translation system). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990).

Oligonucleotides and polynucleotides may optionally include one or more non-standard nucleotide(s), nucleotide analog(s) and/or modified nucleotides. Examples of modified nucleotides include, but are not limited to diaminopurine, S2T, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine and the like. Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone.

As utilized herein, a “deletion” means the removal of one or more nucleotides relative to the native polynucleotide sequence. The engineered strains that are disclosed herein may include a deletion in one or more genes (e.g., a deletion in gmd and/or a deletion in waaL). Preferably, a deletion results in a non-functional gene product. As utilized herein, an “insertion” means the addition of one or more nucleotides to the native polynucleotide sequence. The engineered strains that are disclosed herein may include an insertion in one or more genes (e.g., an insertion in gmd and/or an insertion in waaL). Preferably, a deletion results in a non-functional gene product. As utilized herein, a “substitution” means replacement of a nucleotide of a native polynucleotide sequence with a nucleotide that is not native to the polynucleotide sequence. The engineered strains that are disclosed herein may include a substitution in one or more genes (e.g., a substitution in gmd and/or a substitution in waaL). Preferably, a substitution results in a non-functional gene product, for example, where the substitution introduces a premature stop codon (e.g., TAA, TAG, or TGA) in the coding sequence of the gene product. In some embodiments, the engineered strains that are disclosed herein may include two or more substitutions where the substitutions introduce multiple premature stop codons (e.g., TAATAA, TAGTAG, or TGATGA).

In some embodiments, the engineered strains disclosed herein may be engineered to include and express one or more heterologous genes. As would be understood in the art, a heterologous gene is a gene that is not naturally present in the engineered strain as the strain occurs in nature. A gene that is heterologous to E. coli is a gene that does not occur in E. coli and may be a gene that occurs naturally in another microorganism or a gene that does not occur naturally in any other known microorganism (i.e., an artificial gene).

### Peptides, Polypeptides, Proteins, and Synthesis Methods

As used herein, the terms “peptide,” “polypeptide,” and “protein,” refer to molecules comprising a chain a polymer of amino acid residues joined by amide linkages. The term “amino acid residue,” includes but is not limited to amino acid residues contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The term “amino acid residue” also may include nonstandard or unnatural amino acids. The term “amino acid residue” may include alpha-, beta-, gamma-, and delta-amino acids.

In some embodiments, the term “amino acid residue” may include nonstandard or unnatural amino acid residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine. The term “amino acid residue” may include L isomers or D isomers of any of the aforementioned amino acids.

Other examples of nonstandard or unnatural amino acids include, but are not limited, to a p-acetyl-L-phenylalanine, a p-iodo-L-phenylalanine, an O-methyl-L-tyrosine, a p-propargyloxyphenylalanine, a p-propargyl-phenylalanine, an L-3-(2-naphthyl)alanine, a 3-methyl-phenylalanine, an O-4-allyl-L-tyrosine, a 4-propyl-L-tyrosine, a tri-O-acetyl-GlcNAcpp-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-bromophenylalanine, a p-amino-L-phenylalanine, an isopropyl-L-phenylalanine, an unnatural analogue of a tyrosine amino acid; an unnatural analogue of a glutamine amino acid; an unnatural analogue of a phenylalanine amino acid; an unnatural analogue of a serine amino acid; an unnatural analogue of a threonine amino acid; an unnatural analogue of a methionine amino acid; an unnatural analogue of a leucine amino acid; an unnatural analogue of a isoleucine amino acid; an alkyl, aryl, acyl, azido, cyano, halo, hydrazine, hydrazide, hydroxyl, alkenyl, alkynl, ether, thiol, sulfonyl, seleno, ester, thioacid, borate, boronate, ufa hor, phosphono, phosphine, heterocyclic, enone, imine, aldehyde, hydroxylamine, keto, or amino substituted amino acid, or a combination thereof, an amino acid with a photoactivatable cross-linker; a spin-labeled amino acid; a fluorescent amino acid; a metal binding amino acid; a metal-containing amino acid; a radioactive amino acid; a photocaged and/or photoisomerizable amino acid; a biotin or biotin-analogue containing amino acid; a keto containing amino acid; an amino acid comprising polyethylene glycol or polyether; a heavy atom substituted amino acid; a chemically cleavable or photocleavable amino acid; an amino acid with an elongated side chain; an amino acid containing a toxic group; a sugar substituted amino acid; a carbon-linked sugar-containing amino acid; a redox-active amino acid; an α-hydroxy containing acid; an amino thio acid; an α,α disubstituted amino acid; a β-amino acid; a γ-amino acid, a cyclic amino acid other than proline or histidine, and an aromatic amino acid other than phenylalanine, tyrosine or tryptophan.

As used herein, a “peptide” is defined as a short polymer of amino acids, of a length typically of 20 or less amino acids, and more typically of a length of 12 or less amino acids (Garrett & Grisham, Biochemistry, 2nd edition, 1999, Brooks/Cole, 110). In some embodiments, a peptide as contemplated herein may include no more than about 2, 3, 4, 5, 6, 7, 8, 9, 10, 11, 12, 13, 14, 15, 16, 17, 18, 19, or 20 amino acids. A polypeptide, also referred to as a protein, is typically of length >100 amino acids (Garrett & Grisham, Biochemistry, 2nd edition, 1999, Brooks/Cole, 110). A polypeptide, as contemplated herein, may comprise, but is not limited to, 100, 101, 102, 103, 104, 105, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or more amino acid residues.

A peptide as contemplated herein may be further modified to include non-amino acid moieties. Modifications may include but are not limited to acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine or histidine).

The terms “antibody” or “antibody molecule” are used herein interchangeably and refer to immunoglobulin molecules or other molecules which comprise an antigen binding domain. The term “antibody” or “antibody molecule” as used herein is thus intended to include whole antibodies (e.g., IgG, IgA, IgE, IgM, or IgD), monoclonal antibodies, chimeric antibodies, humanized antibodies, and antibody fragments, including single chain variable fragments (ScFv), single domain antibody, and antigen-binding fragments, genetically engineered antibodies, among others, as long as the characteristic properties (e.g., ability to bind RNA-DNA hyrbids) are retained.

As stated above, the term “antibody” includes “antibody fragments” or “antibody-derived fragments” and “antigen binding fragments” which comprise an antigen binding domain. The term “antibody fragment” as used herein is intended to include any appropriate antibody fragment that displays antigen binding function, for example, Fab, Fab′, F(ab′)2, scFv, Fv, dsFv, ds-scFv, Fd, dAbs, TandAbs dimers, mini bodies, monobodies, diabodies, and multimers thereof and bispecific antibody fragments.

### Methods and Systems

As discussed above, in various aspects, methods are disclosed for detecting one or more human papillomaviruses (IPV) in a biological sample.

The biological sample can be any type of biological sample. In various aspects, the biological sample can include any bodily fluid or tissue. In certain aspects, the biological sample can include a biopsy specimen. In various aspects, the biopsy specimen can be from a subject having a tumor or suspected tumor and/or the biopsy specimen can include at least a portion of a tumor or suspected tumor. In one or more aspects, the biopsy specimen can include a cervical biopsy specimen and/or an oropharyngeal biopsy specimen. In various aspects, the biological sample can be prepared for use in an enzyme-linked immunosorbent assay (ELISA), immunohistochemistry, microscopy, fluorescent microscopy, or a combination thereof. In one example aspect, the biological sample may be a frozen section biopsy specimen. In the same or alternative aspects, the biological sample may be a permanent section biopsy specimen.

In various aspects, the methods can include exposing the biological sample to an agent that binds to DNA-RNA hybrids and/or R-loops. In various aspects, the agent can be an antibody or antibody fragment, RNaseH or modified version thereof, such as an enzymatically inactive version, RNA polymerase, including bacterial and eukaryotic versions, such as RNA Polymerase II, and RNA Polymerase III. In various aspects, the antibody or fragment can be any antibody or fragment that is capable of binding to DNA-RNA hybrids and/or R-loops. In one aspect, the antibody or antibody fragment can bind to DNA-RNA hybrids and/or R-loops of a length of from about 5 base pairs to about 50 base pairs, of from about 5 base pairs to about 30 base pairs, or of from about 8 base pairs to about 25 base pairs. In various aspects, the antibody or antibody fragment can include the S9.6 antibody or Fab fragment.

In one or more aspects, the methods can further include detecting the binding of the agent, e.g., antibody or antibody fragment, to DNA-RNA hybrids and/or R-loops in the biological sample. In various aspects, such a step can include any convenient method for detecting binding of an agent to DNA-RNA hybrids and/or R-loops. For instance, in one aspect, such a step can include the use of an enzyme-linked immunosorbent assay (ELISA), immunohistochemistry, microscopy, fluorescent microscopy, or a combination thereof.

In various aspects, the methods can include determining that the biological sample contains HPV based on detecting the binding of the agent, e.g., antibody or antibody fragment, to DNA-RNA hybrids and/or R-loops in the biological sample. In one or more aspects, such a step can include comparing a level of binding of the antibody or antibody fragment to DNA-RNA hybrids in the biological sample to a level of binding of the antibody or antibody fragment to DNA-RNA hybrids and/or R-loops in a control sample. The control sample can be a sample that includes tissue from the subject but from a region not having a tumor or suspected tumor. The control sample can be a sample that includes tissue from a control subject who does not have an HPV infection. In one or more aspects, the comparison can include comparing results for the control and the biological sample from an ELISA, immunohistochemistry, microscopy, fluorescent microscopy, or a combination thereof. In one aspect, a level of binding of the antibody, antibody fragment, or agent can be quantified. In various aspects, determining that the biological sample contains HPV based on detecting the binding of the agent, e.g., antibody or antibody fragment, to DNA-RNA hybrids and/or R-loops in the biological sample, and can include comparing the level of binding to a threshold value. In various aspects, a threshold value can be a level of detected binding observed in a control sample, and can vary based on the method of detection. In some embodiments, the presence of HPV in the biological sample is indicated by a level of binding observed in the biological sample that is at least 0.1-fold, 0.5-fold, 1-fold, 5-fold, 10-fold, 25-fold, 50-fold, 100-fold, 500-fold, or 1000-fold greater than the level of binding observed in the control sample, or is within a range bounded by any of the forgoing. In some embodiments, the determining is based on the total level of binding to DNA-RNA hybrids and/or R-loops observed in the biological sample. In some embodiments, the determining is based on level of binding to DNA-RNA hybrids and/or R-loops observed at a specific gene locus. In some embodiments, the gene locus is MYADM, RPL13a, SLC35B2, LGAL2, or ALU elements.

In one or more aspects, the methods can include analyzing a biological sample for the presence of the p16 protein and for the presence of DNA-RNA hybrids and/or R-loops. Detecting the presence of p16 protein in a biological sample is a current method for identifying HPV positive biological samples and/or biopsy specimens. In one aspect, the method can include exposing a portion of a biological sample to an antibody or antibody fragment that binds to p16 and detecting binding of the antibody or antibody fragment to the biological sample. The biological sample can include any or all of the properties and/or parameters discussed above. In one or more aspects, an antibody or antibody fragment that binds to p16 can be any convenient antibody or fragment that binds to p16 and may be commercially obtained. In various aspects, the method may include exposing another portion of the biological sample to an antibody or antibody fragment that binds to DNA-RNA hybrids, and detecting binding of the antibody or antibody fragment to DNA-RNA hybrids in that portion of the biological sample. In such aspects, the detection of DNA-RNA hybrids can be used to confirm p16 positive results and/or detection of p16 can be used to confirm R-loop positive results (e.g., via detection of DNA-RNA hybrids). In one or more aspects, the detection of DNA-RNA hybrids in a biological sample is more sensitive than current methods for detection of p16.

In various aspects, the methods can include therapeutically treating a subject. In such aspects, the subject can be therapeutically treated based on determining that the biological sample contains HPV, which can be based on detecting the binding of the agent, e.g., antibody or antibody fragment, to DNA-RNA hybrids and/or R-loops in the biological sample. In various aspects, the therapeutic treatment can include removal of the tumor or suspected tumor and/or administering one or more therapeutic agents.

### Exemplary Embodiments

Embodiment 1. A method for detecting one or more human papillomaviruses (HPV) in a biological sample, comprising:
exposing a biological sample to an antibody or antibody fragment that binds to DNA-RNA hybrids;
detecting binding of the antibody or antibody fragment to DNA-RNA hybrids in the biological sample; and
determining that the biological sample contains HPV based on the detecting binding of the antibody or antibody fragment to DNA-RNA hybrids in the biological sample.

Embodiment 2. The method of embodiment 1, wherein the antibody or antibody fragment comprises a S9.6 antibody.

Embodiment 3. The method of embodiment 1 or 2, wherein the biological sample comprises a biopsy specimen.

Embodiment 4. The method of embodiment 3, wherein the biopsy specimen is a cervical biopsy specimen or an oropharyngeal biopsy specimen.

Embodiment 5. The method of any one of embodiments 1-4, wherein the biological sample is from a subject having a tumor or suspected tumor, and wherein the biological sample comprises at least a portion of the tumor or suspected tumor.

Embodiment 6. The method of any one of embodiments 1-5, wherein the determining comprises comparing a level of binding of the antibody or antibody fragment to DNA-RNA hybrids in the biological sample to a level of binding of the antibody or antibody fragment to DNA-RNA hybrids in a control sample.

Embodiment 7. The method of embodiment 6, wherein the control sample comprises tissue from the subject from a region not having a tumor or suspected tumor.

Embodiment 8. The method of any one of embodiments 1-7, wherein the detecting binding of the antibody or antibody fragment to DNA-RNA hybrids in the biological sample comprises the use of an enzyme-linked immunosorbent assay (ELISA), immunohistochemistry, fluorescent microscopy, or a combination thereof.

Embodiment 9. The method of embodiment 5, further comprising therapeutically treating the subject, based on the determining that the biological sample contains HPV.

Embodiment 10. The method of embodiment 9, wherein the therapeutically treating the subject comprises removing the tumor or suspected tumor and/or administering one or more anti-cancer therapeutic agents.

Embodiment 11. A method for detecting one or more human papillomaviruses (HPV) in a biological sample, comprising:
exposing a first portion of the biological sample to an antibody or antibody fragment that binds to p16 protein;
detecting binding of the antibody or antibody fragment to p16 in the first portion of the biological sample;
exposing a second portion of the biological sample to an antibody or antibody fragment that binds to DNA-RNA hybrids; and
detecting binding of the antibody or antibody fragment to DNA-RNA hybrids in the second portion of the biological sample.

Embodiment 12. The method of embodiment 11, further comprising determining that the biological sample contains HPV based on: the detecting binding of the antibody or antibody fragment to p16 in the first portion of the biological sample; and the detecting binding of the antibody or antibody fragment to DNA-RNA hybrids in the second portion of the biological sample.

Embodiment 13. The method of embodiment 11 or 12, wherein the biological sample comprises a biopsy specimen.

Embodiment 14. The method of embodiment 13, wherein the biopsy specimen is a cervical biopsy specimen or an oropharyngeal biopsy specimen.

Embodiment 15. The method of any one of embodiments 11-14, wherein the biological sample is from a subject having a tumor or suspected tumor, and wherein the biological sample comprises at least a portion of the tumor or suspected tumor.

Embodiment 16. The method of any one of embodiments 11-15, wherein the antibody or antibody fragment that binds to DNA-RNA hybrids comprises a S9.6 antibody.

Embodiment 17. The method of embodiment 12, wherein the determining comprises comparing a level of binding of the antibody or antibody fragment to DNA-RNA hybrids in the second portion of the biological sample to a level of binding of the antibody or antibody fragment to DNA-RNA hybrids in a control sample.

Embodiment 18. The method of embodiment 17, wherein the control sample comprises tissue from the subject from a region not having a tumor or suspected tumor.

Embodiment 19. The method of any one of embodiments 11-18, wherein the detecting binding of the antibody or antibody fragment to DNA-RNA hybrids in the second portion of the biological sample comprises the use of an enzyme-linked immunosorbent assay (ELISA), immunohistochemistry, fluorescent microscopy, or a combination thereof.

Embodiment 20. The method of embodiment 12, further comprising therapeutically treating the subject based on the determining that the biological sample contains HPV.

Embodiment 21. The method of embodiment 20, wherein therapeutically treating the subject comprises removing the tumor or suspected tumor and/or administering one or more anti-cancer therapeutics.

### EXAMPLES

The following Examples are illustrative and should not be interpreted to limit the scope of the claimed subject matter.

### Example 1

### Summary

R-loops are trimeric RNA:DNA hybrids that are important physiological regulators of transcription; however, their aberrant formation or turnover leads to genomic instability and DNA breaks. High-risk human papillomaviruses are the causative agents of genital as well as oropharyngeal cancers and exhibit enhanced amounts of DNA breaks. The levels of R-loops were found to be increased up to 50-fold in cells that maintain high-risk HPV genomes and were readily detected in squamous cell cervical carcinomas in vivo but not in normal cells. The high levels of R-loops in HPV positive cells were present on both viral and cellular sites together with RNase H1, an enzyme that controls their resolution. Depletion of RNase H1 in HPV positive cells further increased R-loop levels, resulting in impaired viral transcription and replication along with reduced expression of the DNA repair genes such as FANCD2 and ATR, both of which are necessary for viral functions. Overexpression of RNase H1 decreased total R-loop levels, resulting in a reduction of DNA breaks by over 50%. Furthermore, increased RNase H1 expression blocked viral transcription and replication while enhancing the expression of factors in the innate immune regulatory pathway. This suggests that maintaining elevated R-loop levels is important for the HPV life cycle. The E6 viral oncoprotein was found to be responsible for inducing high levels of R-loops by inhibiting p53's transcriptional activity. The data presented herein indicates that high R-loop levels are involved in HPV pathogenesis and that this depends on suppressing the p53 pathway.

High-risk human papillomaviruses (HPV) are the etiological agents of genital and oropharyngeal cancers. Although prophylactic vaccines are effective in blocking initial infection by these viruses, they are not effective against existing lesions. Understanding the mechanisms regulating HPV pathogenesis is therefore important for the identification of new biomarkers and for the development of novel therapeutics. The data presented herein demonstrates that high levels of trimeric RNA:DNA structures called R-loops are present in HPV positive cells derived from low-grade cervical lesions as well as in squamous cell carcinomas. These elevated R-loop levels are necessary for both viral gene expression and DNA replication. The date presented herein demonstrate that R-loops can function as cellular regulators of HPV pathogenesis, and they may be useful as novel biomarkers for viral infection or therapeutic targets.

As discussed above, R-loops are trimeric structures consisting of an RNA-DNA hybrid and a displaced DNA strand that are formed during transcription [1-3]. These structures form at promoters and sites of termination to regulate transcription [4-6]; however, aberrant R-loop formation or turnover can lead to genomic instability and DNA breaks [7, 8]. R-loop homeostasis is maintained by enzymes such as RNase H1 and H2 as well as senataxin [9]. RNase H1 can degrade the RNA moiety in R-loops and controls the formation of aberrant R-loops [10, 11]. Similarly, senataxin resolves R-loops that form at the 3′ end of the transcribed genes [12, 13]. In normal cells, R-loops function in regulating transcription, DNA damage repair, and other functions, while in cancers, they can result in genomic instability.

Human papillomaviruses are the causative agents of cervical and most oropharyngeal cancers [14-17]. HPVs infect cells in the basal layer of stratified epithelia and establish their genomes as nuclear episomes at about 100 copies per cell [18, 19]. Initial studies indicated that the levels of R-loops were increased in HPV positive cells but whether this had an effect on viral pathogenesis was unclear [20]. In precancerous lesions, HPV genomes are maintained at a constant copy number in basal cells and replicated simultaneously with cellular DNA [21]. As HPV positive cells migrate from the basal layer they re-enter S/G2 in suprabasal layers, where productive replication occurs in a process called amplification [22]. Both stable maintenance replication and amplification depend on activation of the ATM and ATR DNA repair pathways by the E6 and E7 viral proteins through induction of high levels of DNA breaks [23, 24]. The preferential and rapid repair of these breaks in HPV DNAs is necessary for viral replication [20]. DNA breaks result from the improper formation or resolution of R-loops but whether they contribute to HPV pathogenesis cells is unknown [25].

In this example, high levels of R-loops were detected on both viral and cellular sequences in cells that stably maintain episomes as well as squamous cell cervical carcinomas. The levels of R-loop regulatory enzymes such as RNase H1 were similarly increased. Knockdown of RNase H1 increased the levels of R-loops and at the same time impaired viral transcription and stable replication of HPV episomes. The resultant increased levels of R-loops also repressed expression of cellular genes involved in DNA damage repair including FANCD2 and ATR both of which are involved in viral replication [26, 27]. The increased levels of R-loops were the result of E6 directed inhibition of p53 function. These studies identify R-loops as regulators of HPV pathogenesis, whose altered homeostasis is dependent upon repression of p53.

### Determining Role of R-Loops in HPV Life Cycle

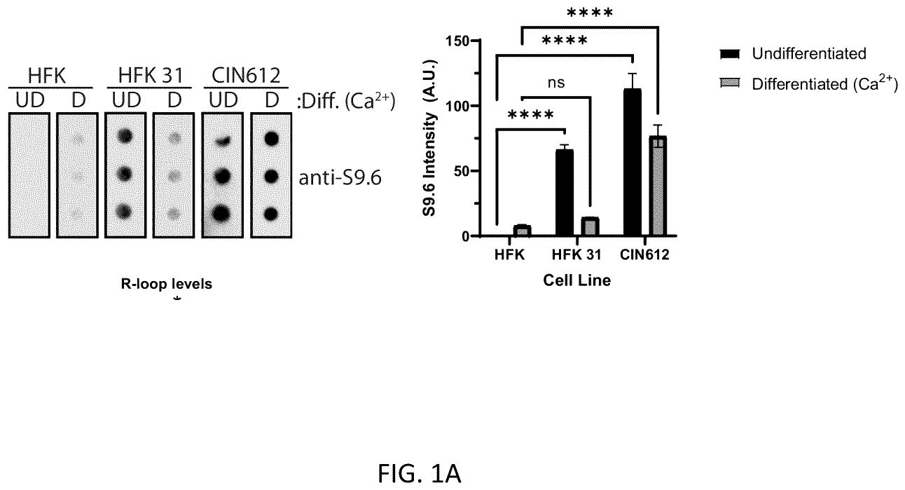

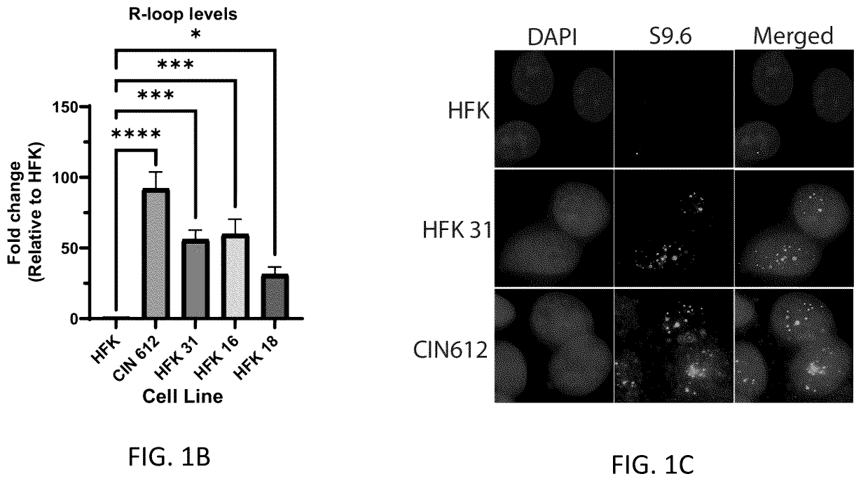

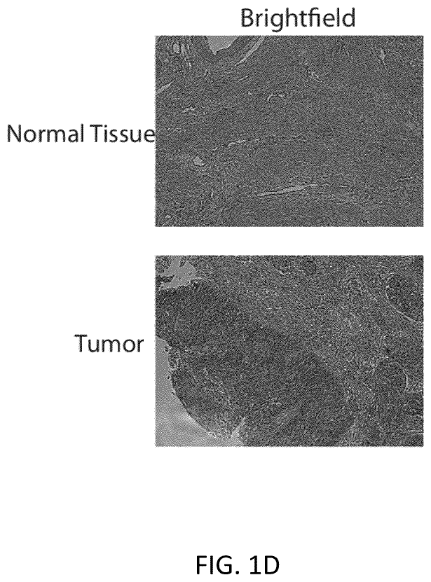

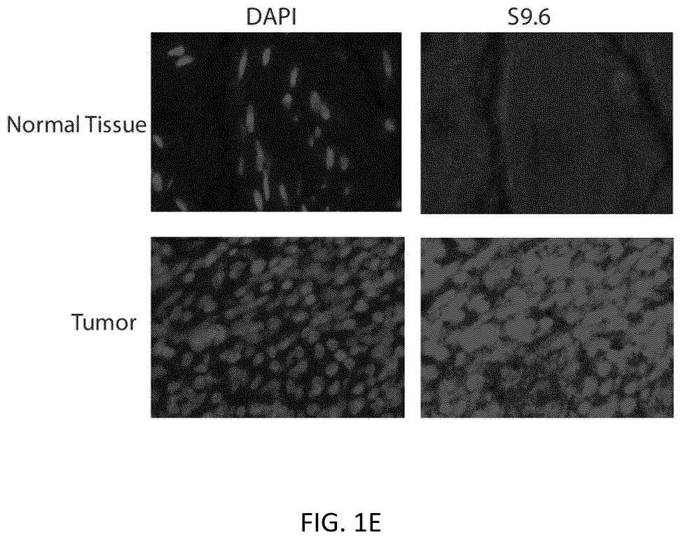

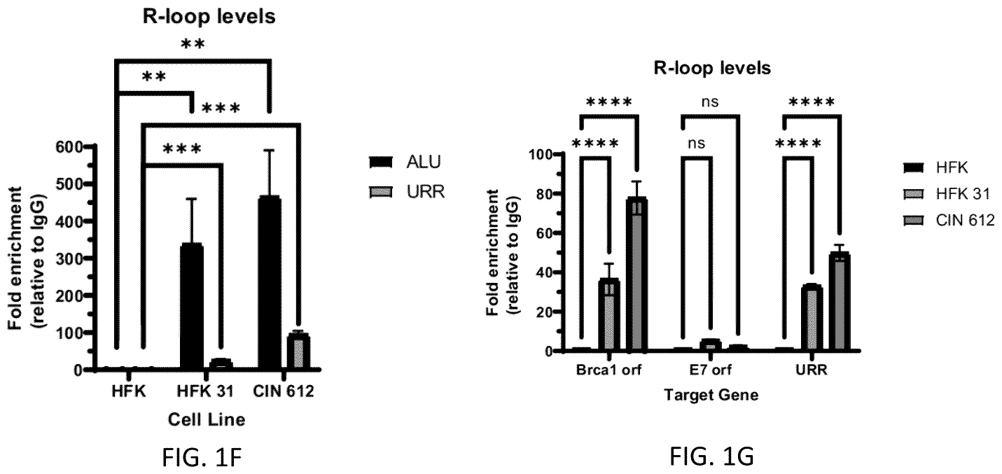

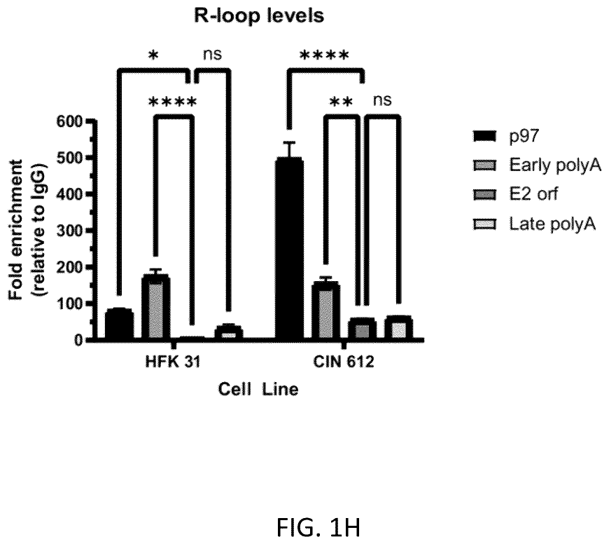

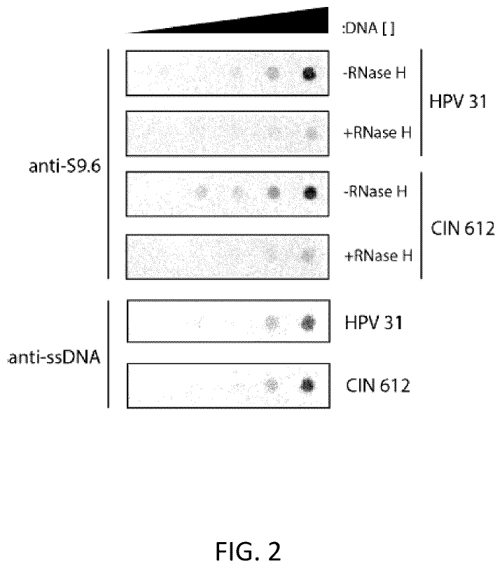

To investigate what role, if any, R-loops might play in the HPV life cycle, the levels were examined in cells that stably maintain high-risk HPV 31 episomes. CIN612 cells were derived from a low-grade CIN biopsy while HFK-31 cells were generated by transfection of cloned viral sequences into primary human keratinocytes (HFK) [28, 29]. DNA-RNA dot blot analysis was performed utilizing an antibody that preferentially recognizes R-loops (S9.6) [30, 31], and 50-100 fold higher levels were detected in HPV positive cells as compared to HFKs (FIG. 1A). In order to confirm that the increase was due to R-loops, samples were treated with RNase H to remove the RNA component prior to spotting on a membrane, and the signal was shown to be specific (FIG. 2). To determine if the presence of high R-loop levels extended to other high-risk types, we performed S9.6 dot blot assays using extracts from cells that stably maintain HPV 16 or 18 episomes and detected levels similar to those seen in HPV 31 positive cells (FIG. 1B). This indicates that high levels of R-loops are present in cells from multiple high-risk HPV types.

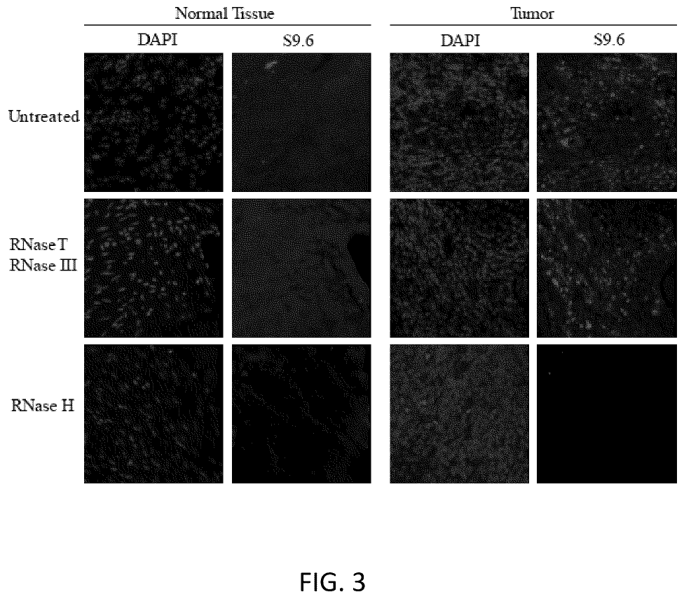

The life cycle of HPV is linked to the differentiation of the host keratinocyte [32], so it was important to determine if levels of R-loops in HFKs, HFK 31, and CIN 612 cells changed upon calcium-induced differentiation (FIG. 1A). The switch from low to high calcium media with keratinocytes grown in monolayer cultures induces differentiation that initiates around 48 hours and peaks at 72 hours [29]. In HPV positive cells, R-loop levels were reduced upon differentiation and this was most pronounced in HFK-31 cells as compared to CIN 612. Interestingly, the levels of R-loops in HFKs were modestly increased upon differentiation in contrast to the decrease seen in HPV positive cells. In this study we focus our analyses on R-loop effects in undifferentiated cells. We next investigated where R-loops were localized in HPV positive cells through immunofluorescence assays using the S9.6 antibody. R-loops were detected in multiple nuclear foci in undifferentiated HPV positive cells, while only a small number of such foci were detected in HFKs (FIG. 1C). High levels of R-loops were also detected in tissue sections from biopsies of squamous cell cervical carcinomas and were absent in adjacent normal tissues (FIG. 1D, 1E). Specificity of the S9.6 antibody in detecting R-loops from crosslinked tissues was addressed by treating tissues with RNase T and RNase III or RNase H and examining the staining of S9.6. In these tissues, S9.6 staining is sensitive to RNase H treatment and minimally to RNase T and III treatment (FIG. 3).

It was next important to determine if R-loops were associated with viral or cellular DNAs through DNA-RNA immunoprecipitation (DRIP) assays. This method uses the S9.6 antibody to precipitate R-loop complexes followed by qPCR for the DNA region of interest to measure binding [33]. The formation of R-loops on the upstream regulatory region (URR) of the HPV genome was examined by DRIP analysis and compared to that seen on cellular sequences using ALU sequences as examples. High levels of R-loops were detected at both the URR of HPV 31 as well as at ALU sequences in both HFK-31 and CIN 612 cells (FIG. 1F). In contrast, very low levels of R-loops were observed at ALU sequences in HFKs. Since ALU sequences are present at high copy numbers within the cell, the presence of R-loops was also examined at promoter sequences around the BRCA1 gene which has previously been reported to maintain R-loops and whose expression is upregulated in HPV positive cancers [34, 35]. R-loops were detected within both coding and promoter regions of BRCA1 in the HFKs but were significantly increased in HPV positive cells (FIG. 1G). These observations indicate that the enhanced levels of R-loops detected in HPV positive cells were not the result of high-level formation only on viral episomes but were increased on cellular sequences as well. We next investigated if R-loops formed uniformly on the HPV genome or whether they were preferentially localized to the URR. For this analysis, R-loop formation within the coding sequences of E7 was compared to its association with the URR and found substantially higher levels present on the latter (FIG. 1H). DRIP analysis was also performed on the late and early polyA sites [36], the E2 orf together with the p97 promoter in the URR (FIG. 1F). Both the early polyA site and the p97 promoter region had significantly more R-loops compared to the E2 orf or the late polyA site. These data indicate that R-loops preferentially form at regions on the viral genome important for transcription of early viral genes.

Undifferentiated HPV-Positive Cells have Increased Levels of Proteins Responsible for R-Loop Resolution

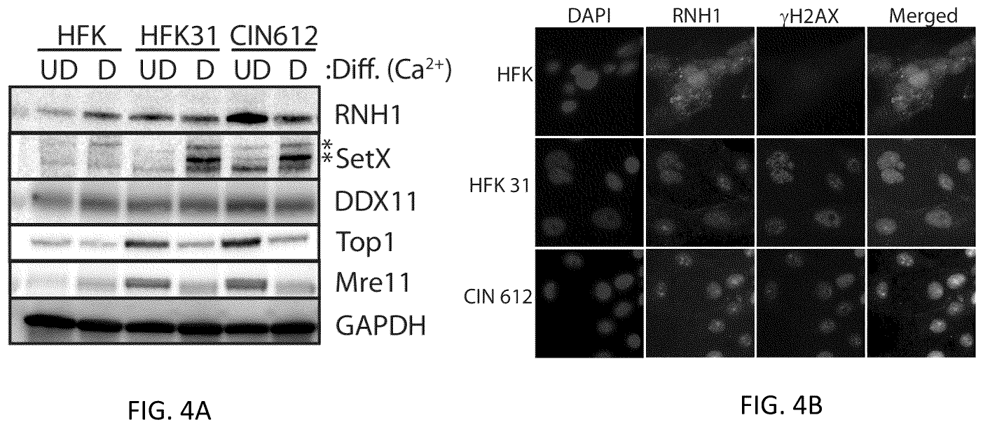

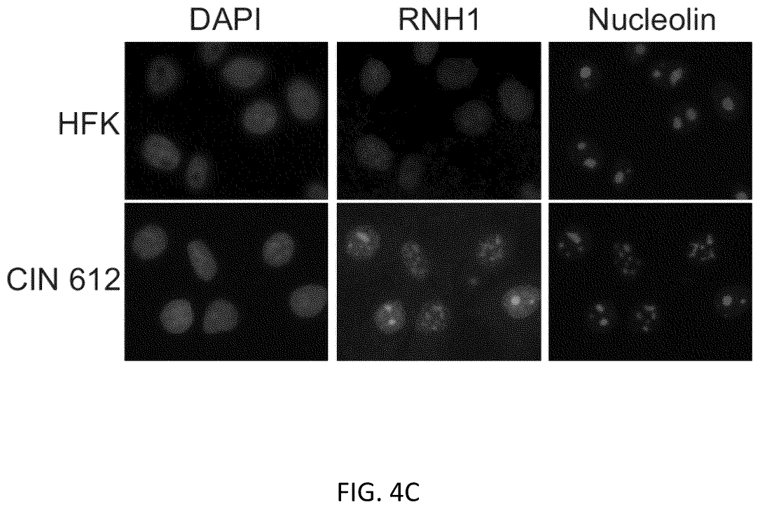

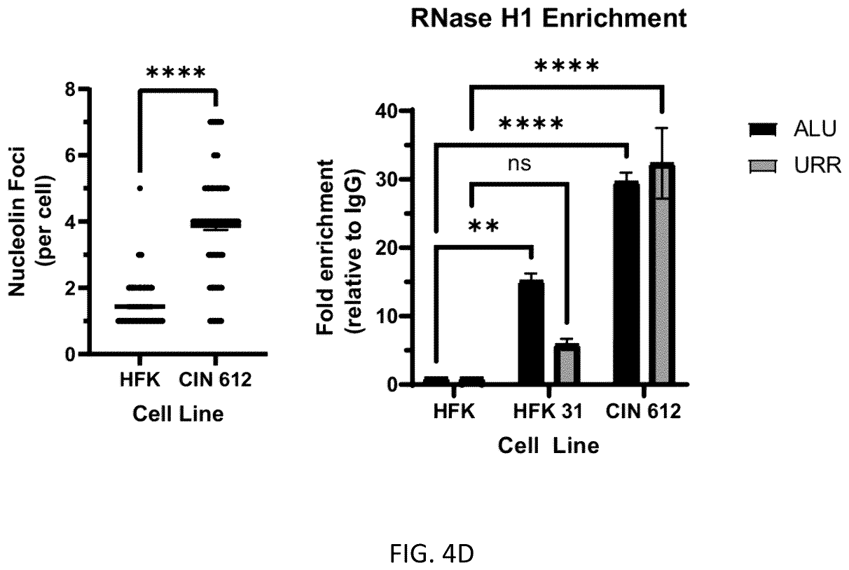

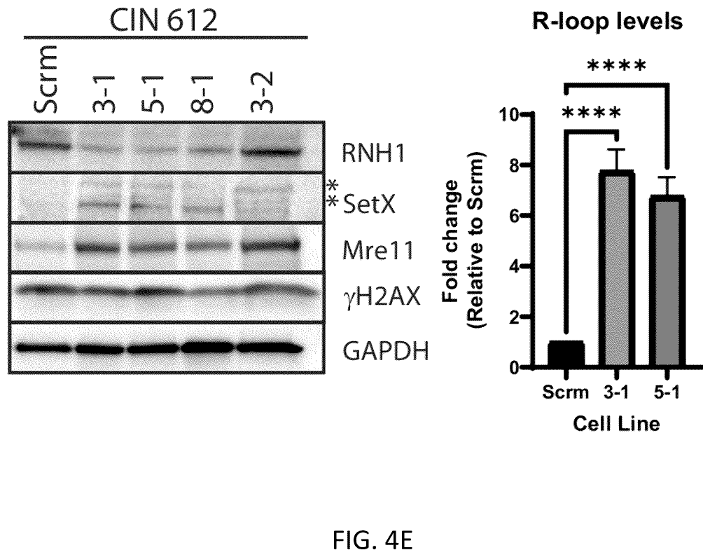

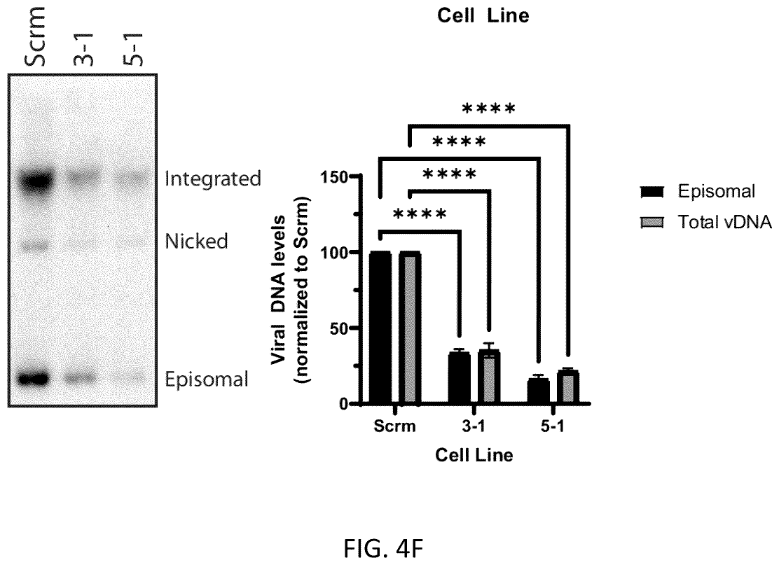

Since the formation and turnover of R-loops is regulated by enzymes such as RNase H1, senataxin, Mre11, DDX11, as well as TOP1 [37], it was important to determine whether the high-levels of R-loops in HPV positive cells was due to a reduction in the levels of these factors. Western blot analysis of undifferentiated HPV-positive cells demonstrated increased levels of all these factors compared to HFKs and paralleled the high levels of R-loops detected in these cells (FIG. 4A). Upon calcium-induced differentiation, the levels of RNase H1, Mre11, DDX1, and Top1 1 decreased in the HPV positive cells to the levels seen in HFKs even though the number of R-loops remained high. Only the levels of senataxin were found to remain elevated upon differentiation. This indicates that increased levels of R-loops in HPV positive cells are not due to decreased steady-state levels of proteins responsible for the resolution of these structures.

RNase H1 is Enriched within the Nucleoli of HPV-Positive Cells

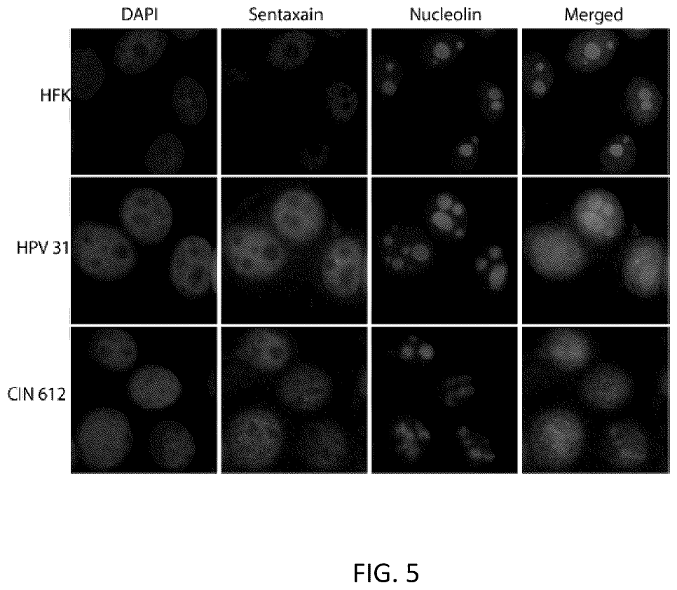

While HPV positive cells maintain a high level of RNase H1, it was possible that its subcellular localization was altered to inhibit its action. Immunofluorescence analysis of RNase H1 in HFKs demonstrated a pan-nuclear distribution with some cytoplasmic localization (FIG. 4B, top row). In CIN612 and HFK-31 cells, RNase H1 also exhibited a pan-nuclear distribution along with a number of densely staining foci. In addition, the total relative intensity of the signal increased compared to HFKs (FIG. 4B, rows 2-3). Since these RNase H1 positive foci resembled nucleoli, immunofluorescence for nucleolin, a marker of nucleoli [38], was performed and identified these areas as nucleoli enriched with RNase H1 (FIG. 4C, left). In addition, approximately 3 times more nucleolin puncta were observed in HPV-positive cells than HFKs (FIG. 4C, right). RNase H1 has been reported to act with RNA poll in mediating rRNA transcription and may explain this localization to nucleoli [39]. In contrast the R-loop regulatory enzyme senataxin exhibited the same subcellular localization in both HFKs and HPV positive cells with no recruitment to nucleoli though again total levels were increased (FIG. 5). R-loops can also be associated with the formation of DNA breaks, but only modest co-localization with γH2AX, a surrogate marker for breaks [40], was observed (FIG. 4B). To determine whether RNase H1 was recruited to viral or cellular genomes, chromatin immunoprecipitation assays were performed. HFK 31 and CIN 612 cells exhibited high levels of RNase H1 on both the HPV URR and ALU sequences with the latter being increased in comparison to the HFKs (FIG. 4D). Interestingly, the amount of RNaseH1 binding per viral genome was significantly higher than the binding per ALU element. ALU elements are present at over 500 thousand copies per cell, while the HPV genomes are maintained at 50-100 copies.

### Depletion of RNase H1 Impairs HPV Genome Maintenance

The presence of high-levels of both RNase H1 and R-loops on viral genomes suggested they may play a role in the HPV life cycle. Therefore, the effect of depleting RNase H1 on viral replication and transcription was investigated. RNase H1 was stably depleted in CIN 612 cells by transduction with lentiviruses expressing shRNAs and western analysis showed levels were reduced by 3-fold relative to the scrambled shRNA control (FIG. 4E). This moderate reduction in RNase H1 levels, however, increased the amounts of R-loop levels by 5 to 10-fold, confirming its functions in the resolution of these structures (FIG. 4E). Since RNase H1 provides essential functions [41], the depleted cells could only be passaged up to 5 times before cell growth was arrested or suppression of RNase H1 expression was lost [10, 42]. We screened for effects on HPV replication by Southern analysis at the second passage after depletion and found HPV genomes were reduced by approximately 4-fold (FIG. 4F). This indicates that stable replication of HPV episomes is impaired as a result of RNase H1 knockdown with the corresponding increase in R-loops.

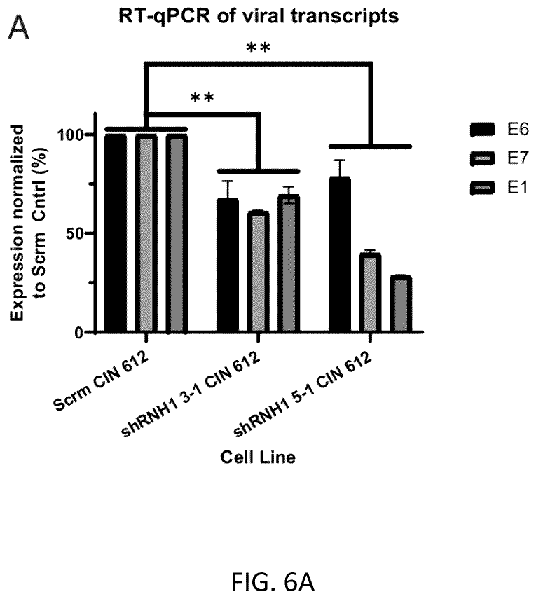

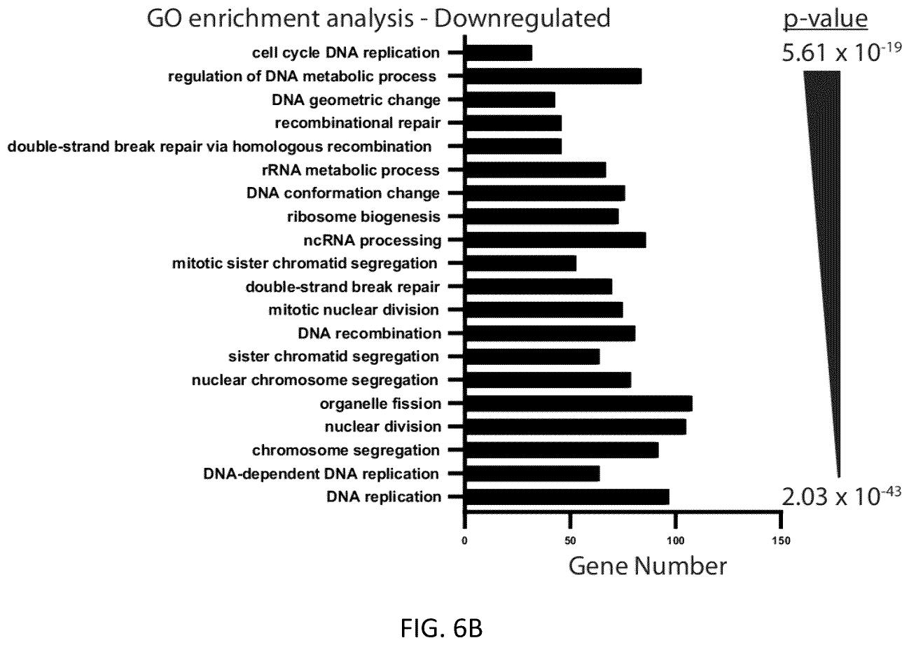

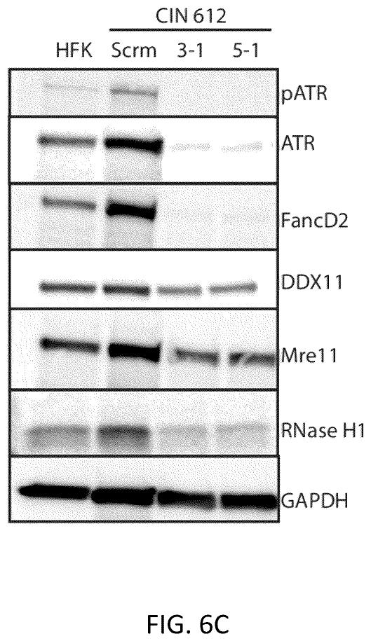

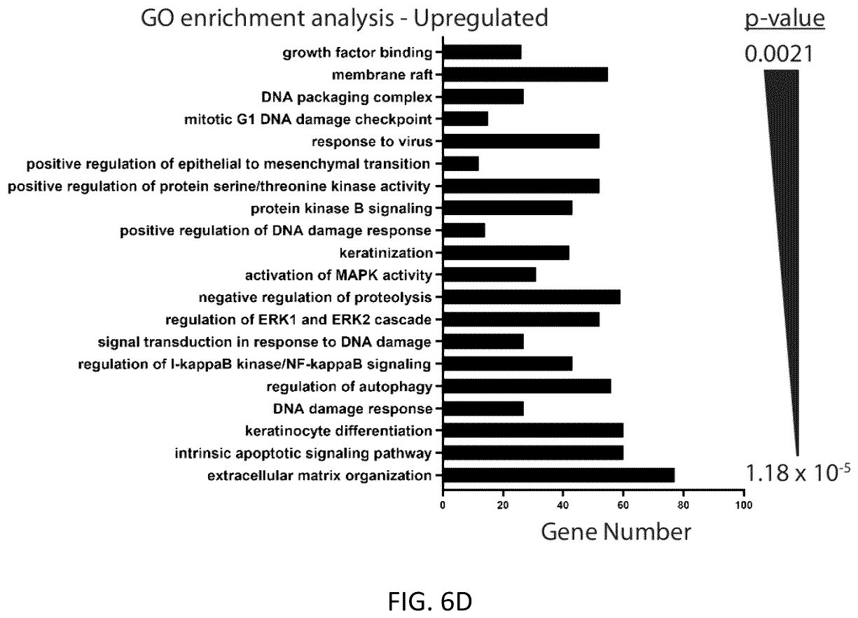

R-loops can positively regulate both initiation and termination of transcription and can decrease levels if improperly formed or resolved. To determine what effect increased levels of R-loops had on viral transcription, RT-qPCR was used to examine levels of E6, E7, and E1 transcripts in undifferentiated CIN 612 cells that were depleted of RNase H1 and compared to the scramble control. Depletion of RNase H1 decreased transcript levels by 30-50% suggesting that viral gene expression correlated with R-loop homeostasis (FIG. 6A).

Depletion of RNase H1, which leads to an increase in R-loops, could also affect cellular gene expression. To investigate how depletion of RNase H1 affected cellular gene expression, RNA-sequencing analysis (RNA-seq) was performed on CIN 612 cells that were depleted of RNase H1. Depletion of RNase H1 affected the expression of a number of cellular pathways including those involved in DNA replication, DNA damage response, and DNA recombination all of which impact the HPV life cycle (FIG. 6B). Of particular interest were reductions in factors such as FANCD2, ATR and Mre11 which have been shown to be important for HPV replication [26, 27, 43]. Western blot analysis confirmed that the reduced transcript levels of FANCD2, ATR, Mre11, and RNase H1 corresponded to lower protein steady-state levels (FIG. 6C). One important factor is FANCD2 as its knockdown impairs HPV replication in undifferentiated cells and may act together with ATR and Mre11 to explain the effects on viral replication upon depletion of RNase H1 [26]. Other genes were upregulated by RNase H1 depletion including those in the p53 arm of the DNA damage response [44], such as GADD45A, MDM2, and p21 (FIG. 6D). In addition, the expression of transcriptional repressors such as SNAIL along with members of the epithelial cell integrity pathway, including KLK5, KLK6, and KLK13 were similarly increased.

### Overexpression of RNase H1 Impair HPV Genome Maintenance

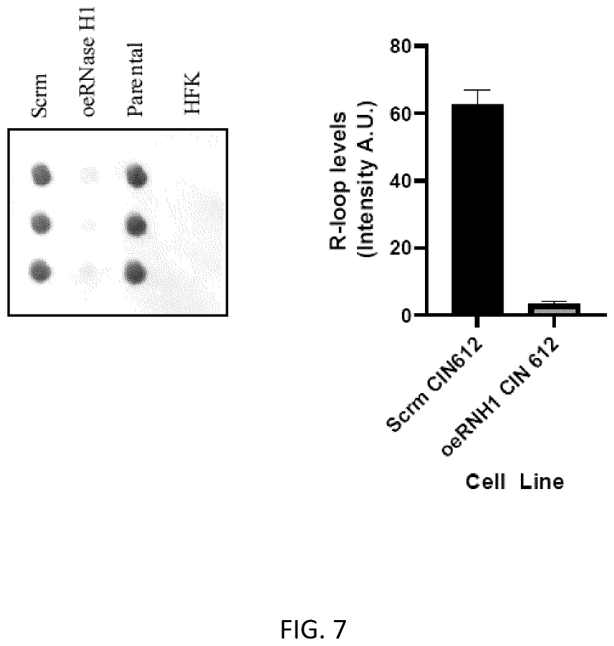

While knockdown of RNase H1 increased the levels of R-loops, overexpression can reduce levels [45]. To investigate how increasing RNase H1 levels impacted viral functions, HPV positive cells were transfected with a vector expressing a GFP-tagged RNase H1 that lacked the N-terminal mitochondrial localization signal (ILS), so it only localized to the nucleus [46]. Increased levels of RNase H1 were confirmed by western analyses and localization to the nucleus was detected by immunofluorescence analyses. In addition, a decrease in the level of R-loops was confirmed by S9.6 dot blot assays (FIG. 7).

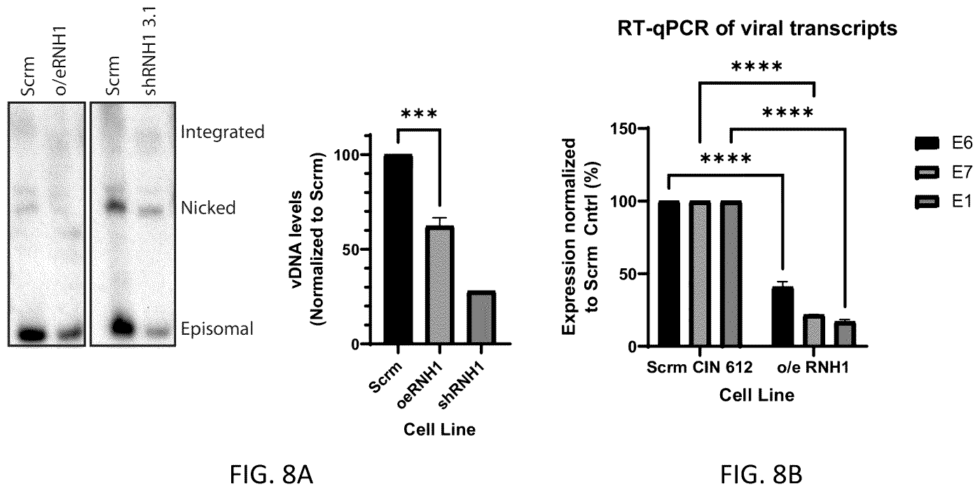

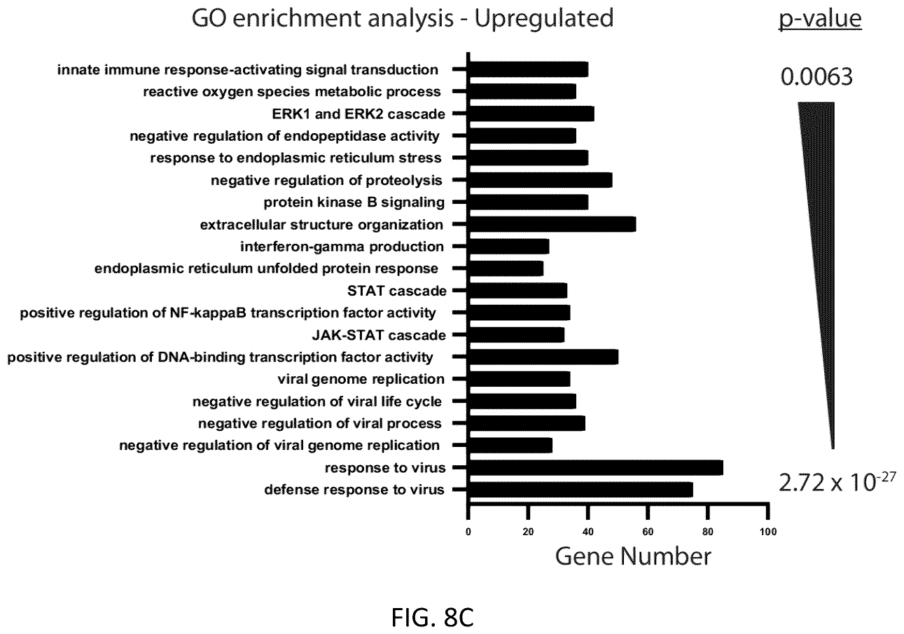

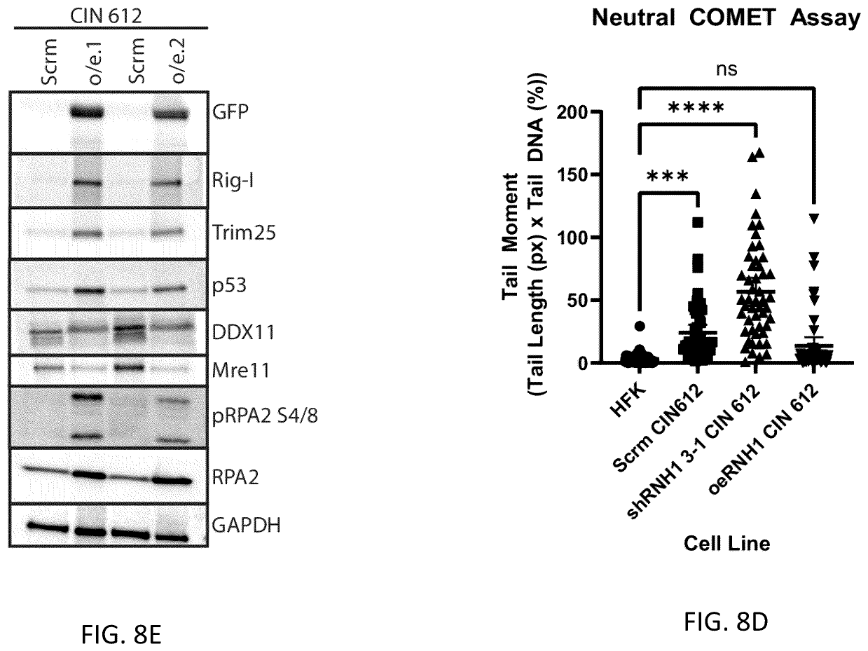

The effect of increased expression of RNase H1 on viral genome maintenance was next examined by Southern blot analysis (FIG. 8A). CIN 612 cells overexpressing RNase H1 substantially reduced viral episomes relative to the scramble control cells after as early as one passage. Furthermore, the levels of viral transcripts for E6, E7 and E1 were reduced by 60 to 80% that of the control CIN 612 cells (FIG. 8B). RNA-seq was performed on RNase H1 overexpressing CIN 612 cells to examine how reducing R-loops affected cellular gene expression. The most significant pathway altered was the innate immune signaling pathway where expression was increased as compared to the control CIN 612 cells and included genes such as DDX58, OASL, TRIM25, IL1A, IFIT2, and IFIT3 (FIG. 8C). In addition, transcriptional regulators such as p73, EGR1, KLF15, STAT4, and E2F2 were found to be downregulated. Western blot analysis confirmed the upregulation of DDX58 and TRIM25 at the protein level; both of which are inhibitory to HPV replication [47](FIG. 8D). These data indicate that overexpression of RNase H1 and the resultant reductions in R-loops within HPV positive cells is important for regulating immune signaling and gene expression.

### DNA Breaks in HPV Positive Cells are Caused by R-Loop Formation

A major activity associated with the aberrant formation or resolution of R-loops is the induction of DNA breaks [20]. HPVs have been shown to induce high levels of DNA breaks in cells which leads to the activation of DNA repair pathways [20], so we investigated if high levels of R-loops could be a major source. For this analysis, COMET assays were performed using CIN612 cells that were either depleted or overexpressing RNase H1 and compared to HFKs and the scramble control CIN 612 cells. The scramble control CIN 612 cells exhibited about an 8-fold greater DNA breaks compared to HFKs, consistent with previous reports (FIG. 8E). When RNase H1 was depleted using shRNAs from CIN 612 cells, there was an approximate 2.5-fold further increase in DNA breaks compared to the scramble control. In contrast, overexpression of RNase H1 reduced breaks over 50%. These data indicate that R-loops provide a source for DNA breaks within HPV positive cells, and that the altered R-loop homeostasis within HPV positive cells is responsible for over 50% of the DNA breaks.

### HPV 31 E6 Induces R-Loop Formation in HFKs

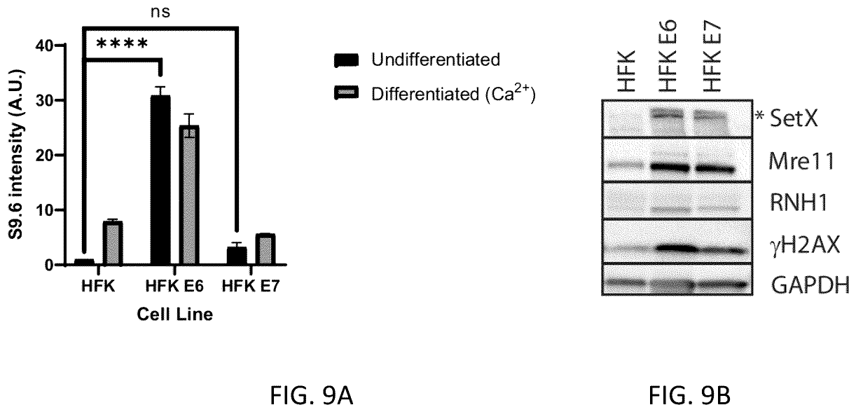

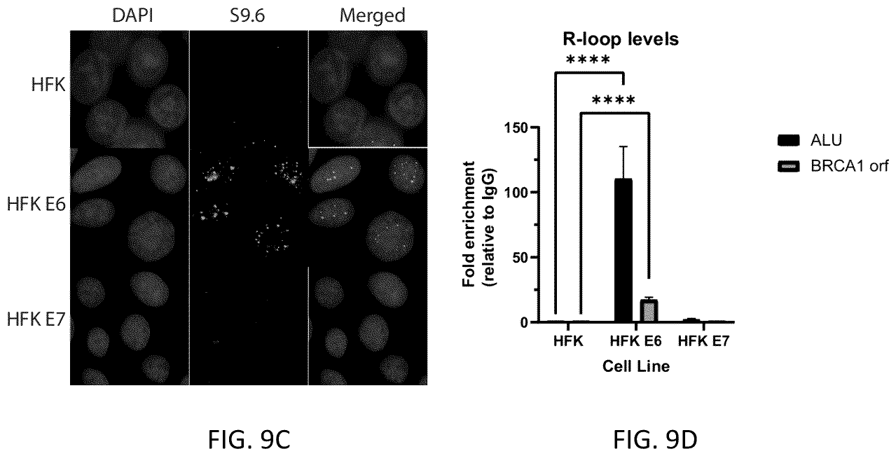

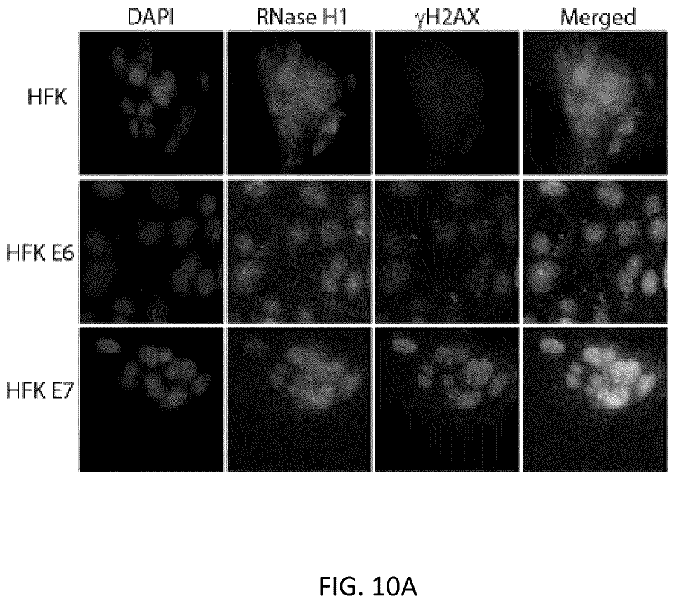

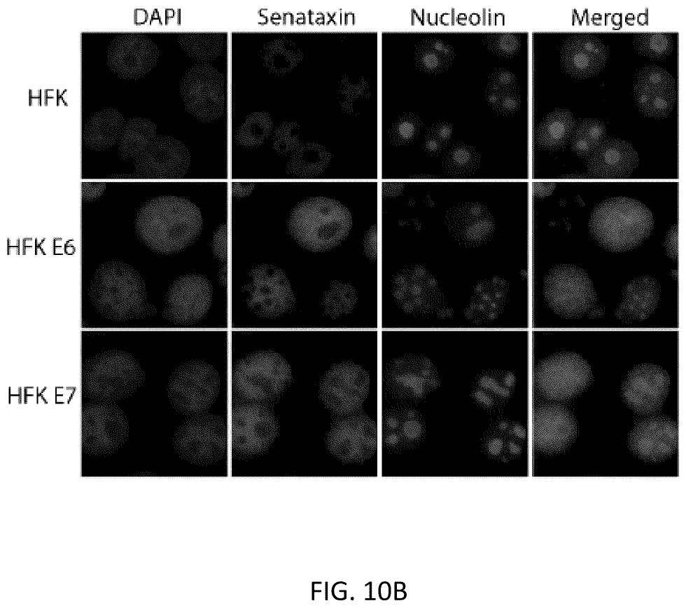

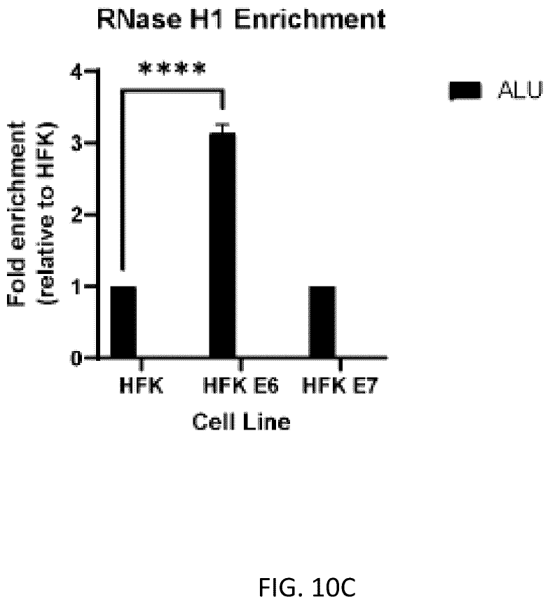

Our studies suggest that the high levels of R-loops in HPV positive cells may provide important functions in viral life cycle, and it was important to determine whether this increase was a result of viral replication or if the expression of viral proteins alone was sufficient. HFKs expressing E6 or E7 were generated through retroviral transduction and examined for the presence of R-loops by S9.6 dot blot analysis (FIG. 9A). The expression of E6 was found to be sufficient to induce high levels of R-loops in both undifferentiated and differentiated states. In contrast, the expression of E7 failed to alter R-loop levels significantly. The levels of R-loops detected in E6-expressing cells were reduced from that seen in cells that stably maintain episomes. This could be due to lower levels of E6 expression in these cells or a contribution from other viral proteins. Interestingly the expression of either E6 or E7 resulted in increased levels of the R-Loop regulatory enzymes senataxin, RNase H1, and Mre11 (FIG. 9B). The increased levels of these proteins in cells expressing E7, which do not have increased R-loop levels, suggest that this is not the sole determining factor regulating R-loop formation. Immunofluorescence analyses using the S9.6 antibody identified that R-loops formed within nuclear puncta of HFKs expressing E6 cells similar to the staining seen within cells with HPV episomes (FIG. 9C), while HFKs expressing E7 more closely resembled the control HFKs. Dot blot analysis with S9.6 antibodies confirmed that HFKs expressing E6 formed significantly more R-loops than control or E7-expressing HFKs (FIG. 9D) and showed increased R-loop formation on BRCA1 as well as ALU sequences. Finally, expression of E6 alone was found to be sufficient to direct recruitment of RNase H1 to the nuclear puncta like that seen in HPV-positive cells, while RNase H1 within E7-expressing HFKs exhibits a pan-nuclear localization (FIG. 10A). The localization of other proteins involved in R-loop resolution like senataxin was however not altered by E6 (FIG. 10B). RNase H1 binding to cellular sequences was approximately 3-fold higher in E6 expressing cells than HFKs expressing E7 or HFKs (FIG. 10C).

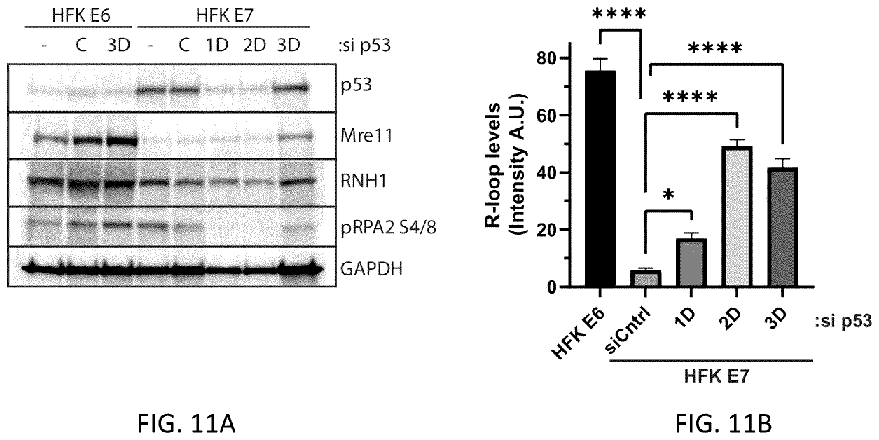

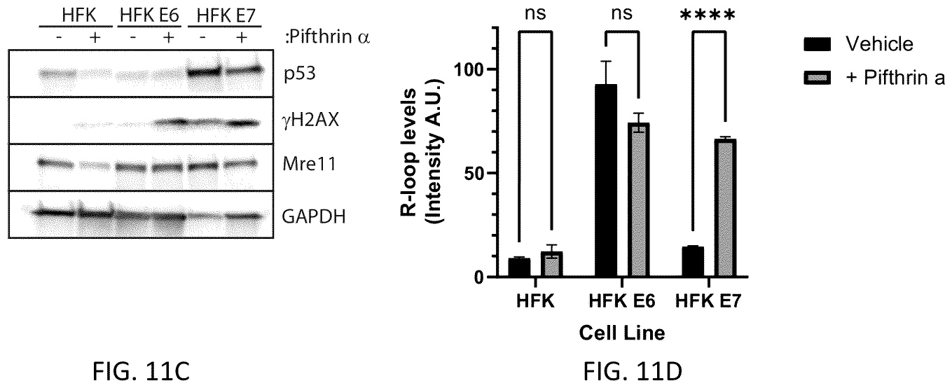

The oncoprotein E6 has many functions, including the capability to degrade and inactivate p53 [48-50]. To determine whether the decreased p53 levels seen in E6 expressing HFKs were responsible for R-loop formation, we transiently depleted p53 using siRNA in HFKs expressing E7, which by themselves exhibit high levels of p53. Depletion of p53 steady state levels was observed for 2 days with a restoration of repression on day 3 (FIG. 11A). Decreasing p53 levels correlated with decreasing levels of several proteins important for R-loop resolution like Mre11 and RNase H1. R-loop levels were significantly increased within these p53 depleted cells after 1 day, peaked on day 2, and decreased slightly on day 3 (FIG. 11B). These data support the hypothesis that inactivating p53 via E6 drives R-loop formation within HPV positive cells.

Pifithrin-α is an inhibitor of p53's transcriptional activity and its effect on R-loop formation in E7 cells was examined [51]. HFK, HFK E6, or HFK E7 cells were treated with pifithrin-α and R-loop levels were assessed by S9.6 dot blot while p53 levels examined by western blot. Consistent with previous findings, E7-expressing cells exhibited high levels of p53 while E6-expressing cells had low levels (FIG. 11C). Treatment of E7 cells with pifithrin-α induced high levels of R-loops. In contrast, inhibition of p53 with pifithrin-α in HFKs did not increase R-loop levels indicating the effect may be specific to E7 expressing cells. These experiments confirm that repression of p53 function is important for induction of high levels of R-loops in HPV positive cells.

### Comparison of p16 Antibody and S9.6 Antibody Staining of Normal and HPV Positive Tumor Tissue

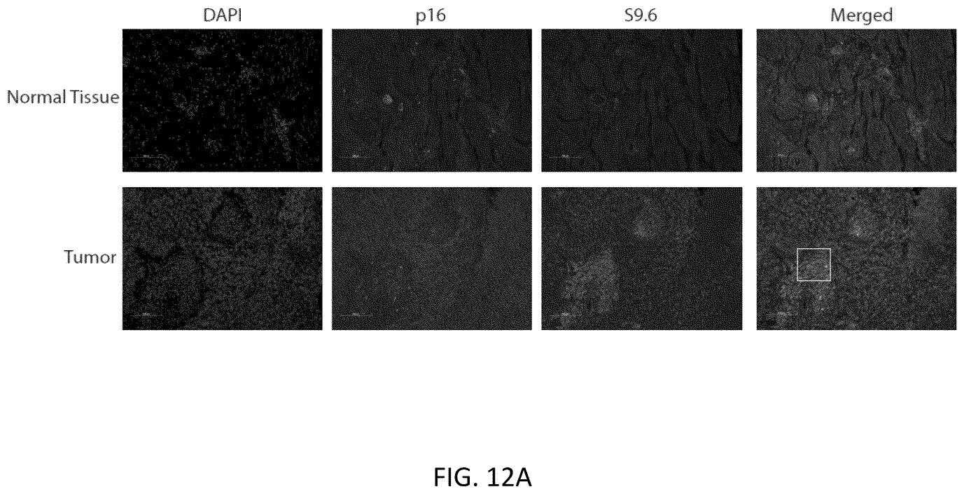

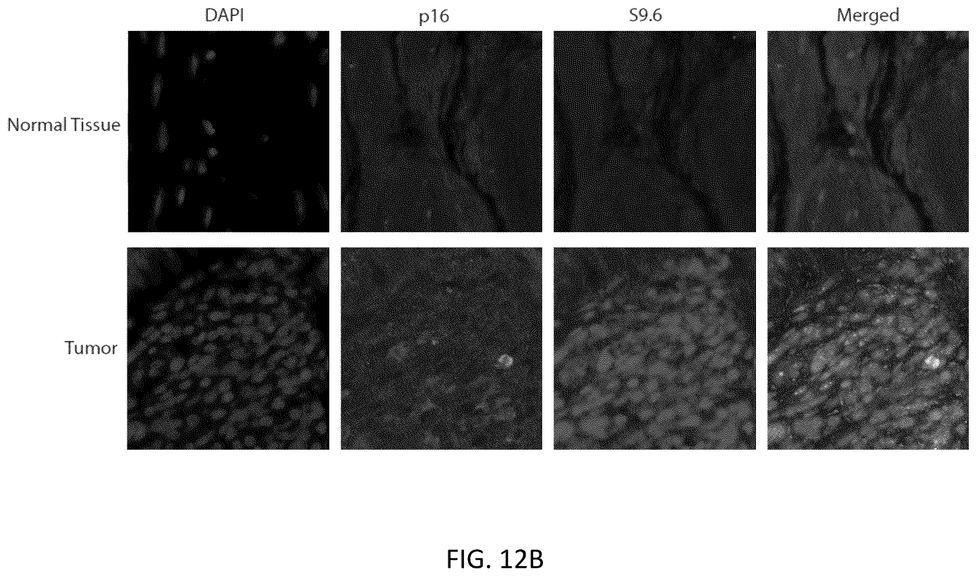

Samples of normal tissue and tumor tissue were labeled with p16 antibody and S9.6 antibody and fluorescently stained for IC analysis (see FIGS. 12A and 12B). S9.6 antibody labeled more cells than the p16 antibody, thus detecting more potential HPV positive cells (see FIG. 12B). Furthermore, the S9.6 label was brighter and sharper compared to p16, making it easier to identify potential HPV positive cells. This increased sensitivity can allow for increased identification of HPV positive tissue, which can more accurately identify the margins of a tumor, e.g., for potential removal.

### Isolation of Human Keratinocytes and Cell Culture

Human keratinocytes were isolated from deidentified neonatal foreskins provided by the Skin Disease and Research Core at Northwestern University as previously described [20]. Cells were cultured as previously described [20, 61, 62]. Briefly, HFKs and CIN 612 cells which were isolated from deidentified biosamples and stably maintain HPV 31 episomes were cocultured in E-media with NIH-3T3-J2 fibroblasts (J2s) which were growth arrested with mitomycin C. J2s and HEK-293T cells were cultured in Dulbecco's modified Eagle's medium (DMEM) with 10% FBS and 1% pen-strep. HFKs stably maintaining HPV 31 episomes were generated as previously described [63]. HFKs stably expressing viral oncogenes E6 or E7 were generated as previously described [20]. Cells were treated with Pifithrin-α (100 μm) for 24 hours to assess the effect of p53 inhibition on R-loop levels.

Generation of Cell Lines that Stably Express shRNAs or RNase H1-eGFP

Plasmids encoding shRNA sequences targeting RNase H1 were purchased from Sigma. The sequences of the RNAs targeted RNase H1 are listed in Table 1. Lentiviruses were generated with each of the four shRNA encoding plasmids in HEK-293T cells using the 2^{nd} generation AddGene system. CIN612 cells were transduced with the various lentiviruses and selected using puromycin (2 g/ml). Depletion of RNase H1 was validated by western blot analysis and fold change was quantified via densitometry using ImageJ (NIH). Overexpression of RNase H1 was achieved by using the pEGFP-RNase H1 vector (Addgene plasmid #108699). Lentiviruses were generated using this vector or empty vector control using the 2^{nd} generation AddGene system in HEK-293T cells. CIN 612 cells were then transduced and assessed for RNase H1 expression by immunofluorescence and western blot analysis of GFP.

TABLE 1
shRNA sequence targeting RNase H1.

| Code (TRC Number) | Sequence |
|---|---|
| shRNH1 3-1 (N0000049783) | 5′-GCCGTATGCAAAGCACATGAA-3′ |
| (SEQ ID NO: 1) | |
| shRNH1 5-1 (N0000049785) | 5′-CCTGGTCATTCGGGATTTATA-3′ |
| (SEQ ID NO: 2) | |
| shRNH1 8-1 (N0000119548) | 5′-CACTCAGGATTTGTGGGCAAT-3′ |
| (SEQ ID NO: 3) | |
| shRNH1 3-2 (N0000291902) | 5′-GCAAAGCCATTGAACAAGCAA-3′ |
| (SEQ ID NO: 4) | |

### Calcium-Induced Differentiation

5×10⁷ cells were collected and plated into 10 cm dishes containing M154 media containing 0.07 mM CaCl₂) supplemented with human keratinocyte growth serum (HKGS) (LifeTech). After 24 hours, the media was changed to that containing 0.03 mM CaCl₂). On the third day, M154 medium without HKGS and containing 1.5 mM CaCl₂) was added to confluent monolayers of keratinocytes. Differentiating keratinocytes were incubated for up to 72 hours at 37° C. before being harvested for downstream analyses. Validation of differentiation was assessed through a comparison of K10 levels between undifferentiated and differentiated cell lysates.

siRNA Transfections

Transient silencing of p53 expression was performed in HFKs expressing either HPV31-E6 or -E7 with transfected siRNAs according to protocols from Santa Cruz Biotechnology. Cell lysates were collected 24 to 96 hours post-transfection and assessed by western blot analysis for p53 steady-state levels and dot blot analysis for R-loops.

### S9.6 Dot-Blot Analysis

DNA was purified from cell lysates using PhenolChloroform extractions and spotted onto a positively charged membrane (Zeta-probe). Membranes were then blocked with 500 BSA in TBST (Tris-buffered saline Tween 20) before being probed with the S9.6 anti-RNA:DNA hybrid antibody (Millipore) overnight at 4° C. The following day, membranes were washed with TBST, probed with secondary antibody for 1 hour at RT, and developed using ECL (Fisher, 4500085). Images were taken using an Odyssey Fc LiCor (LiCor BioSciences).

### Western and Southern Blot Analysis

Western blot analysis was performed as previously described [61] using the antibodies listed in Table 2. Southern blot analysis was performed as previously described [61].

TABLE 2

| | | Dilution | Dilution for | |
|---|---|---|---|---|
| Antibody | Host Animal | for IF | Western blot | Distributor, cat. No. |
| RNase H1 | Rabbit, | 1:200 | 1:1000 | ThermoFisher, |
| | polyclonal | | | 15606-1-AP |
| RNase H1 | Mouse, | N/A | 1:100 | Santa Cruz Biotechnology, |
| | monoclonal | | | sc-365267 |
| ssDNA | Mouse, | N/A | 1:1000 | Millipore Sigma, MAB3868 |
| | monoclonal | | | |
| DNA-RNA Hybrid | Mouse, | 1:200 | 1:1000 | Millipore Sigma, MABE1095 |
| (S9.6) | monoclonal | | | |
| Senataxin | Rabbit, | N/A | 1:1000 | Novus Biologicals, NBP194712 |
| | polyclonal | | | |
| GAPDH | Mouse, | N/A | 1:4000 | Santa Cruz Biotechnology, |
| | monoclonal | | | sc47742 |
| Mre11 | Rabbit, | 1:400 | 1:1000 | Cell Signaling Technologies, |
| | polyclonal | | | 4896S |
| DDX11 | Mouse, | N/A | 1:1000 | Santa Cruz Biotechnology, |
| | monoclonal | | | sc271711 |
| Mouse IgG | Mouse, | 1:50 | N/A | Fischer Scientific, |
| | monoclonal | | | C1540000115 |
| GFP (B-2) | Mouse, | N/A | 1:200 | Santa Cruz Biotechnology, |
| | monoclonal | | | sc9996 |
| pATR (ser1981) | Rabbit, | N/A | 1:500 | Cell Signaling Technologies, |
| | polyclonal | | | 13050S |
| ATR | Mouse, | 1:400 | 1:100 | Santa Cruz Biotechnologies, |
| | monoclonal | | | sc515173 |
| Nucleolin | Mouse, | N/A | N/A | Invitrogen, |
| | monoclonal | | | 39-6400 |
| γH2AX (ser139) | Rabbit, | 1:200 | 1:1000 | Cell Signaling Technologies, |
| | monoclonal | | | 9718S |
| γH2AX | Mouse, | N/A | N/A | Fisher, 05-636-IMI |
| | monoclonal | | | |
| pRP A32 (ser8) | Rabbit, | N/A | 1:500 | Cell Signaling Technologies, |
| | polyclonal | | | 83745S |
| RP A32 | Rabbit, | N/A | 1:1000 | Abcam, ab2175 |
| | monoclonal | | | |
| FancD2 | Rabbit, | N/A | 1:500 | Abcam, 178705 |
| | monoclonal | | | |
| TRIM25 | Rabbit, | N/A | 1:1000 | Abcam, ab167154 |
| | polyclonal | | | |
| Rig-I | Rabbit, | N/A | 1:1000 | Cell Signaling Technologies, |
| | monoclonal | | | 3734S |
| Anti-Rabbit IgG, | | N/A | 1:3000- | Cell Signaling Technologies, |
| HRP-linked | | | 1:5000 | 7074 |
| Anti-Mouse IgG, | | N/A | 1:3000- | Cell Signaling Technologies, |
| HRP-linked | | | 1:5000 | 7076 |
| Goat anti-Mouse IgG | | 1:400 | N/A | ThermoFisher, |
| (H + L) Alexa Fluor ™ | | | | A-11032 |
| 594 | | | | |
| Goat anti-Rabbit IgG | | 1:400 | N/A | Invitrogen, |
| (H + L) Cross-Adsorbed | | | | A-11008 |
| Secondary Antibody, | | | | |
| Alexa Fluor ™ 488 | | | | |
| Goat anti-Mouse IgG | | 1:400 | N/A | Invitrogen, |
| (H + L) Superclonal ™ | | | | A28175 |
| Recombinant | | | | |
| Secondary Antibody, | | | | |
| Alexa Fluor ™ 488 | | | | |
| Goat anti-Rabbit IgG | | 1:400 | N/A | Invitrogen, |
| (H + L) Highly | | | | A32740 |
| Cross-adsorbed | | | | |
| secondary antibody, | | | | |
| Alexa Fluor ™ Plus 594 | | | | |

### Immunofluorescence Analysis

2.25×10⁵ cells were plated onto a 4-chamber slide (MatTek). The following day, cells were either fixed with 4% paraformaldehyde or methanol and stored in PBS overnight at 4 C. Chambers were permeabilized in 0.5% TritonX-100 and blocked with 3% BSA in PBS. Samples were probed with antibodies listed in supplemental FIG. 1 before staining with DAPI and secondary antibodies. After mounting with mounting medium (VectaShield), chambers were imaged with a Ti2 eclipse (Nikon).

### Immunofluorescence Staining of Paraffin Sections

Six cross sections of the same tissue from high grade cervical carcinomas (n=3) were formalin-fixed and paraffin embedded. IHC was also performed to identify the margins between normal tissue and tumor. Immunofluorescence of paraffin embedded sections was performed as previously described [64]. Heat antigen retrieval was performed at 60° C. overnight. Specificity of S9.6 staining was analyzed by digesting dewaxed, permeabilized tissues with RNase T and III (2.5 U, Invitrogen and ThermoFisher) or RNase H (2.5 U, ThermoFisher) for 1 hour.

### RT-qPCR Analysis

RNA was extracted using the Qiagen RNeasy Kit from confluent 10 cm dishes. Reverse transcription reactions were then performed on 20 ng of RNA using the iScript cDNA Synthesis Kit (BioRad). Real-time PCR was performed using a LightCycler 480 system (Roche) with primer sets mapping to the E1, E6, and E7 open reading frame (Table 3).

TABLE 3
Primer sets used.

| Primer Name | Sequence (5′ to 3′) |
|---|---|
| ALU forward (SEQ ID NO: 5) | ACG AGG TCA GGA GAT CGA GA |
| ALU Reverse (SEQ ID NO: 6) | CTC AGC CTC CCA AGT AGC TG |
| URR Forward (SEQ ID NO: 7) | GAT GCA GTA GTT CTG CGG TTT |
| URR Reverse (SEQ ID NO: 8) | TAT GTT GGC AAG GTG TGT TAG G |
| E6 Forward (SEQ ID NO: 9) | GAC CTC GGA AAT TGC |
| E6 Reverse (SEQ ID NO: 10) | AAC ATG CTA TGC AAC GTC CTG |
| E7 Forward (SEQ ID NO: 11) | AAT TAC CCG ACA GCT CAG ATG |
| E7 Reverse (SEQ ID NO: 12) | GGC ACA CGA TTC CAA ATC AC |
| E2 Forward (SEQ ID NO: 13) | TAC TGT TGT GGA AGG GCA AG |
| E2 Reverse (SEQ ID NO: 14) | TCC CAG CAA AGG ATA TTT CGT C |
| El Forward (SEQ ID NO: 15) | GAC AGA CAG ACA GGG G |
| El Reverse (SEQ ID NO: 16) | CCC GCT GTC TGG AAG TTC |
| Late PolyA Forward (SEQ ID NO: 17) | GCG TGT GTA CTT GTA |
| Late PolyA Reverse (SEQ ID NO: 18) | GCA ACC GAA AAC GGT TAG G |
| P97 Forward (SEQ ID NO: 19) | GGG AGT GAC CGA AAC TGG |
| P97 Reverse (SEQ ID NO: 20) | CGT GTG GTG TGT CGT CC |
| Early PolyA Forward (SEQ ID NO: 21) | GGT ATT GGT ATT GGT ATT GG |
| Early PolyA Reverse (SEQ ID NO: 22) | ACC CAT ACT ACC ATA CCT TA |
| BRCA1 Forward (SEQ ID NO: 23) | GGC TTG TAA CAG CTA CCC TTC |
| BRCA1 Reverse (SEQ ID NO: 24) | CTT CTG GAT TCT GGC TTA TAG GG |

### Neutral Comet Assay

COMET assays were performed following the manufacturer's instructions (Trevigen, cat. No. 4250-050-K). Briefly, ˜50,000 cells were combined with low melt agarose and spread across the CometSlide. Once dry, cells were lysed for 1 hour at 4° C. Cells were equilibrated to 1× neutral electrophoresis buffer and DNA was resolved on an electrophoresis slide tray for 45 min at 21 V, 4° C. DNA was precipitated and stained with SYBR Gold for 30 min before imaging on a Ti2 Eclipse microscope (Nikon). Tail moments were calculated using the open-source software, CometScore 2.0 (% DNA in tail×tail length=tail moment).

### Chromatin Immunoprecipitation Assays

Cells from a confluent 10 cm dish were crosslinked with 1% formaldehyde and collected in RIPA buffer. Samples were analyzed as previously described [20]. Primers used for qPCR analysis are listed (Table 3).

### DNA:RNA Immunoprecipitation (DRIP Assays)

1×10⁷ cells were harvested and collected in Southern lysis buffer before being treated with RNase A (5 ng/ml) and Proteinase K (7.5 ng/ml) at 37° C. overnight. DNA was purified from these samples using phenol-chloroform extractions and 25-50 μg of DNA was used for each sample. DNA was sheared using a Bioruptor (Diagenode) on high power, 30s on/90s off cycles for 20 min or digested using 1 U of mung bean nuclease for 1 hr at 37° C. Input DNA was removed before loading the samples into pre-blocked magnetic beads in IP buffer containing 2 μg of the RNA:DNA hybrid antibody. Immunoprecipitations were allowed to incubate overnight at 4° C. while rotating. The next day, samples were washed 8 times with RIPA buffer for 5 min while rotating. One wash in TE buffer was performed before samples were eluted for 10 min at 65 C in 10% SDS, 10 mM Tris pH 7.4, 50 mM EDTA. DNA was purified from these elutions using a PCR purification kit (Qiagen) and stored at −20° C. Samples were then analyzed by qPCR (primers used listed in Table 3).

### RNA-Sequencing Analysis

1×10⁷ keratinocytes were harvested and analyzed by Admera Biosciences (NJ), who performed the RNA extraction as well as sequencing. Following RNA extraction, mRNA was sequenced using the Illumina platform. Data analysis was also performed by Admera Biosciences (NJ). DeSeq2 reads of genes differentially expressed between control CIN 612 cells and CIN 612 cells depleted of or overexpressing RNase H1 are provided (see Appendix 1 and Appendix 2, respectively). Biostatistical analyses were performed by Admera Biosciences (NJ) services. RNA-sequencing data was deposited to the GEO database (NCBI).

### Statistical Analyses

Statistical analysis was performed using student T-tests and multiple-way ANOVAs on GraphPad Prism9 software (CA, USA). Graph preparation was performed using GraphPad Prism9 software.

### Exemplary Advantages

The data presented herein shows that up to 50-fold higher levels of R-loops are present in cells that maintain high-risk HPV episomes as compared to primary keratinocytes. These R-loops are formed not only on viral genomes but also on cellular sequences such as repetitive ALU elements and regulatory elements for genes such as BRCA1 [52, 53]. The sites at which R-loops form are similar to those detected in normal cells. In HPV positive cells, these high levels of R-loops were found to correlate with efficient viral replication and transcription. HPV oncoproteins induce high levels of DNA breaks to activate ATM and ATR damage repair pathways to facilitate viral replication and our studies indicate that over 50% of these breaks are associated with R-loop formation. The increased levels of R-loops are not only seen in cells with HPV episomes but also in squamous cell cervical cancers in vivo. These structures, therefore, provide important functions in the pathogenesis of HPV infections.

The formation and resolution of R-loops is mediated by enzymes such as RNase H1, senataxin, DDX11, and Mre11. The enhanced levels of R-loops present in HPV positive cells is, however, not the result of reduced levels of these enzymes as they are also increased in these cells. RNase H1 exhibited a pan-nuclear distribution in HFKs and, while this was also seen in HPV positive cells, it was also present in puncta as well as at high levels in nucleoli. Interestingly, HPV positive cells also exhibited increased numbers of nucleoli that contained RNase H1, which may reflect a role of RNase H1 in cooperating with Poll in directing ribosomal RNA transcription [39]. Furthermore, R-loops were detected bound to HPV genomes at the early promoter (p97) and polyA site but not to E7 or E2 coding sequences. RNase H1 was also present at sites with enriched R-loops levels (ALU sequences and the URR), implicating RNase H1 as actively regulating viral and cellular R-loops within these cells.

A reduction in p53 levels was found to be responsible for inducing high levels of R-loops. While E6 and E7 can cause DNA breaks [20], only E6 was able to induce high levels of R-loops. A primary function of E6 is the impairment of p53 function and our studies demonstrate that knockdown of p53 in cells expressing only E7 leads to induction of high levels of R-loops, implicating it as a key regulator. p53 has been reported to regulate the levels of methyl donor S-adenosylmethione (SAM) which in turn controls histone H3 lysine methylation at repetitive satellite DNAs [54]. In p53 deficient pancreatic cancer cells, SAM is repressed resulting in R-loop formation at these repetitive sites, but whether this is a primary mode of action in HPV positive cells is unclear. While E6 expressed from a retroviral promoter was sufficient to induce R-loop formation, the levels were reduced from that seen in cells that maintain complete viral episomes, which may indicate lower expression of E6 from integrated transgenes compared to episomes or that another viral factor also contributes.

The enhanced levels of RNase H1 which resulted in high R-loops were found to be involved in HPV replication and transcription. RNase H1 removes the RNA moiety from the RNA:DNA hybrid of an R-loop and knockdown with shRNAs leads to increased R-loop levels. It is not possible to directly alter R-loop levels, and this can only be achieved by modulating the amounts of its regulatory enzymes such as RNase H1. Changing the level of RNase H1 has been characterized in numerous studies as the gold standard method to modulate R-loops [55-57]. In HPV positive cells, depletion of RNase H1 increased total R-loop levels by ˜8-fold and this resulted in greater amounts of DNA breaks along with impaired viral replication and gene expression. R-loops have been shown to regulate chromatin organization by modulating histone methylation patterns which in turn modulates transcription [58, 59]. In our studies, reducing RNase H1 led to enhanced levels of R-loops and decreased expression of cellular genes, particularly those in DNA damage repair pathways. This included FANCD2 which has been shown to be necessary for viral replication [26]. FANCD2 binds to HPV promoter sequences and has also been shown to form complexes with R-loops suggesting that its association with viral genomes may be mediated through these structures [26]. Another factor whose expression was reduced by the high levels of R-loops was the DNA repair kinase, ATR, whose activation has been shown to be necessary for viral replication [27]. Despite the presence of higher levels of DNA breaks in RNase H1 knockdown cells, activation of repair pathways was not seen due to repressed transcription of certain DNA damage repair genes. Finally viral gene expression, including that of E1, was also reduced by the reductions in RNase H1 and high amounts of R-loops, further contributing to impaired viral replication. These experiments identify multiple factors regulated by RNase H1 and R-loops that are involved in viral gene expression and replication.

While knockdown of RNase H1 leads to increased amounts of R-loops, overexpression reduced levels only slightly higher than those observed in normal HFKs. This reduction in R-loops from RNase H1 overexpression correlated with decreased viral transcription and episomal copy numbers along with increased expression of cellular genes responsible for innate immune signaling. In addition, the levels of DNA breaks were decreased by ˜50%, which resulted in reduced activation of DNA repair pathways. Previous studies have shown that a substantial number of breaks result from the action of topoisomerases such as TOP20 and our studies indicate that a substantial part of the remainder are due to R-loops [60]. The impairment observed in viral gene expression correlated with a reduction in R-loops and suggests that forming these structures on HPV episomes is important for viral transcription. Alternatively, it is possible that increased expression of cellular immune response genes like Rig I and TRIM25 act to hinder viral transcription [47]. Reducing or increasing R-loop levels impairs viral transcription and replication, directly or indirectly, by altering the expression of important cellular genes. Overall, this indicates that R-loop homeostasis in HPV positive cells can be involved in regulating the viral life cycle.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

### REFERENCES

1. Allison, D. F. and G. G. Wang, R-loops: formation, function, and relevance to cell stress. Cell Stress, 2019. 3(2): p. 38-46.
2. Marnef, A. and G. Legube, R-loops as Janus-faced modulators of DNA repair. Nature Cell Biology, 2021. 23(4): p. 305-313.
3. Aguilera, A. and T. Garcia-Muse, R Loops: From Transcription Byproducts to Threats to Genome Stability. Molecular Cell, 2012. 46(2): p. 115-124.
4. Niehrs, C. and B. Luke, Regulatory R-loops as facilitators of gene expression and genome stability. Nat Rev Mol Cell Biol, 2020. 21(3): p. 167-178.
5. Belotserkovskii, B. P., et al., R-loop generation during transcription: Formation, processing and cellular outcomes. DNA Repair (Amst), 2018. 71: p. 69-81.
6. Lee, C.-Y, et al., R-loop induced G-quadruplex in non-template promotes transcription by successive R-loop formation. Nature Communications, 2020. 11(1): p. 3392.
7. Hamperl, S., et al., Transcription-Replication Conflict Orientation Modulates R-Loop Levels and Activates Distinct DNA Damage Responses. Cell, 2017. 170(4): p. 774-786.e19.
8. Arab, K., et al., GADD45A binds R-loops and recruits TET1 to CpG island promoters. Nat Genet, 2019. 51(2): p. 217-223.
9. Petermann, E., L. Lan, and L. Zou, Sources, resolution and physiological relevance of R-loops and RNA-DNA hybrids. Nature Reviews Molecular Cell Biology, 2022. 23(8): p. 521-540.
10. Lockhart, A., et al., RNase H1 and H2 Are Differentially Regulated to Process RNA-DNA Hybrids. Cell Reports, 2019. 29(9): p. 2890-2900.e5.
11. Mazina, O. M., et al., Replication protein A binds RNA and promotes R-loop formation. Journal of Biological Chemistry, 2020. 295(41): p. 14203-14213.
12. Jurga, M., et al., USP11 controls R-loops by regulating senataxin proteostasis. Nat Commun, 2021. 12(1): p. 5156.
13. Cohen, S., et al., Senataxin resolves RNA:DNA hybrids forming at DNA double-strand breaks to prevent translocations. Nat Commun, 2018. 9(1): p. 533.
14. Zhou, C. and J. L. Parsons, The radiobiology of HPV-positive and HPV-negative head and neck squamous cell carcinoma. Expert Reviews in Molecular Medicine, 2020. 22: p. e3.
15. Pytynia, K. B., K. R. Dahlstrom, and E. M. Sturgis, Epidemiology of HPV-associated oropharyngeal cancer. Oral Oncol, 2014. 50(5): p. 380-6.
16. Chaturvedi, A. K., et al., Worldwide trends in incidence rates for oral cavity and oropharyngeal cancers. Journal of Clinical Oncology, 2013. 31(36): p. 4550-4559.
17. Bravo, I. G. and M. Félez-Sánchez, Papillomaviruses: Viral evolution, cancer and evolutionary medicine. Evol Med Public Health, 2015. 2015(1): p. 32-51.
18. Moody, C., Mechanisms by which HPV Induces a Replication Competent Environment in Differentiating Keratinocytes. Viruses, 2017. 9(9).
19. Albert, E. and L. Laimins, Regulation of the Human Papillomavirus Life Cycle by DNA Damage Repair Pathways and Epigenetic Factors. Viruses, 2020. 12(7).
20. Mehta, K., et al., Human Papillomaviruses Preferentially Recruit DNA Repair Factors to Viral Genomes for Rapid Repair and Amplification. mBio, 2018. 9(1): p. e00064-18.
21. Della Fera, A. N., et al. Persistent Human Papillomavirus Infection. Viruses, 2021. 13, DOI: 10.3390/v13020321.
22. Doorbar, J., et al., The Biology and Life-Cycle of Human Papillomaviruses. Vaccine, 2012. 30: p. F55-F70.
23. Moody, C. A. and L. A. Laimins, Human papillomaviruses activate the ATM DNA damage pathway for viral genome amplification upon differentiation. PLoS Pathog, 2009. 5(10): p. e1000605.
24. Hong, S., et al., STAT-5 Regulates Transcription of the Topoisomerase 11-Binding Protein 1 (TopBP1) Gene To Activate the ATR Pathway and Promote Human Papillomavirus Replication. mBio, 2015. 6(6): p. e02006-15.
25. Kaminski, P., et al., Topoisomerase 2&#x3b2; Induces DNA Breaks To Regulate Human Papillomavirus Replication. mBio, 2021. 12(1): p. e00005-21.
26. Spriggs, C. C., et al., FANCD2 Binds Human Papillomavirus Genomes and Associates with a Distinct Set of DNA Repair Proteins to Regulate Viral Replication. mBio, 2017. 8(1): p. e02340-16.
27. Hong, S., et al., STAT-5 Regulates Transcription of the Topoisomerase II&#x3b2;-Binding Protein 1 (TopBP1) Gene To Activate the ATR Pathway and Promote Human Papillomavirus Replication. mBio, 2015. 6(6): p. e02006-15.
28. Bedell, M. A., et al., Amplification of human papillomavirus genomes in vitro is dependent on epithelial differentiation. J Virol, 1991. 65(5): p. 2254-60.
29. Moody, C. A. and L. A. Laimins, Human Papillomaviruses Activate the ATM DNA Damage Pathway for Viral Genome Amplification upon Differentiation. PLOS Pathogens, 2009. 5(10): p. e1000605.
30. Hu, Z., et al., An antibody-based microarray assay for small RNA detection. Nucleic Acids Res, 2006. 34(7): p. e52.
31. Boguslawski, S. J., et al., Characterization of monoclonal antibody to DNA.RNA and its application to immunodetection of hybrids. J Immunol Methods, 1986. 89(1): p. 123-30.
32. Frattini, M. G., H. B. Lim, and L. A. Laimins, In vitro synthesis of oncogenic human papillomaviruses requires episomal genomes for differentiation-dependent late expression. Proc Natl Acad Sci USA, 1996. 93(7): p. 3062-7.
33. Sanz, L. A. and F. Chédin, High-resolution, strand-specific R-loop mapping via S9.6-based DNA-RNA immunoprecipitation and high-throughput sequencing. Nat Protoc, 2019. 14(6): p. 1734-1755.
34. Kono, T., et al., Activation of DNA damage repair factors in HPV positive oropharyngeal cancers. Virology, 2020. 547: p. 27-34.
35. San Martin Alonso, M. and S. M. Noordermeer, Untangling the crosstalk between BRCA1 and R-loops during DNA repair. Nucleic Acids Res, 2021. 49(9): p. 4848-4863.
36. Johansson, C. and S. Schwartz, Regulation of human papillomavirus gene expression by splicing and polyadenylation. Nature Reviews Microbiology, 2013. 11(4): p. 239-251.
37. Petermann, E., L. Lan, and L. Zou, Sources, resolution and physiological relevance of R-loops and RNA-DNA hybrids. Nat Rev Mol Cell Biol, 2022. 23(8): p. 521-540.
38. Biggiogera, M., et al., Nucleolar distribution of proteins B23 and nucleolin in mouse preimplantation embryos as visualized by immunoelectron microscopy. Development, 1990. 110(4): p. 1263-70.
39. Shen, W., et al., Dynamic nucleoplasmic and nucleolar localization of mammalian RNase H1 in response to RNAP I transcriptional R-loops. Nucleic Acids Res, 2017. 45(18): p. 10672-10692.
40. Kinner, A., et al., Gamma-H2AX in recognition and signaling of DNA double-strand breaks in the context of chromatin. Nucleic Acids Res, 2008. 36(17): p. 5678-94.
41. Cerritelli, S. M., et al., Failure to Produce Mitochondrial DNA Results in Embryonic Lethality in Rnaseh1 Null Mice. Molecular Cell, 2003. 11(3): p. 807-815.
42. Holt, I. J., The Jekyll and Hyde character of RNase H1 and its multiple roles in mitochondrial DNA metabolism. DNA Repair (Amst), 2019. 84: p. 102630.
43. Anacker, D. C., et al., Productive replication of human papillomavirus 31 requires DNA repair factor Nbs1. J Virol, 2014. 88(15): p. 8528-44.
44. Williams, A. B. and B. Schumacher, p53 in the DNA-Damage-Repair Process. Cold Spring Harb Perspect Med, 2016. 6(5).
45. Maul, R. W., et al., R-Loop Depletion by Over-expressed RNase H1 in Mouse B Cells Increases Activation-Induced Deaminase Access to the Transcribed Strand without Altering Frequency of Isotype Switching. J Mol Biol, 2017. 429(21): p. 3255-3263.
46. Bubeck, D., et al., PCNA directs type 2 RNase H activity on DNA replication and repair substrates. Nucleic Acids Res, 2011. 39(9): p. 3652-66.
47. Chiang, C., et al., The Human Papillomavirus E6 Oncoprotein Targets USP15 and TRIM25 To Suppress RIG-I-Mediated Innate Immune Signaling. J Virol, 2018. 92(6).
48. Howie, H. L., R. A. Katzenellenbogen, and D. A. Galloway, Papillomavirus E6 proteins. Virology, 2009. 384(2): p. 324-334.
49. Werness, B. A., A. J. Levine, and P. M. Howley, Association of human papillomavirus types 16 and 18 E6 proteins with p53. Science, 1990. 248(4951): p. 76-9.
50. Scheffner, M., et al., The E6 oncoprotein encoded by human papillomavirus types 16 and 18 promotes the degradation of p53. Cell, 1990. 63(6): p. 1129-36.
51. Zhu, J., et al., Pifithrin-α alters p53 post-translational modifications pattern and differentially inhibits p53 target genes. Sci Rep, 2020. 10(1): p. 1049.
52. Hatchi, E., et al., BRCA1 recruitment to transcriptional pause sites is required for R-loop-driven DNA damage repair. Mol Cell, 2015. 57(4): p. 636-647.
53. Bai, X., F. Li, and Z. Zhang, A hypothetical model of trans-acting R-loops-mediated promoter-enhancer interactions by Alu elements. J Genet Genomics, 2021. 48(11): p. 1007-1019.
54. Panatta, E., et al., Metabolic regulation by p53 prevents R-loop-associated genomic instability. Cell Reports, 2022. 41(5): p. 111568.
55. Cerritelli, S. M., K. Sakhuja, and R. J. Crouch, RNase H1, the Gold Standard for R-Loop Detection. Methods Mol Biol, 2022. 2528: p. 91-114.
56. Shen, W., et al., Dynamic nucleoplasmic and nucleolar localization of mammalian RNase H1 in response to RNAP I transcriptional R-loops. Nucleic Acids Research, 2017. 45(18): p. 10672-10692.
57. Parajuli, S., et al., Human ribonuclease H1 resolves R-loops and thereby enables progression of the DNA replication fork. J Biol Chem, 2017. 292(37): p. 15216-15224.
58. Ginno, P. A., et al., R-loop formation is a distinctive characteristic of unmethylated human CpG island promoters. Mol Cell, 2012. 45(6): p. 814-25.
59. Hartono, S. R., I. F. Korf, and F. Chédin, GC skew is a conserved property of unmethylated CpG island promoters across vertebrates. Nucleic Acids Res, 2015. 43(20): p. 9729-41.
60. Hong, S., et al., Topoisomerase 11-binding protein 1 activates expression of E2F1 and p73 in HPV-positive cells for genome amplification upon epithelial differentiation. Oncogene, 2019. 38(17): p. 3274-3287.
61. Gusho, E. and L. A. Laimins, Human papillomaviruses sensitize cells to DNA damage induced apoptosis by targeting the innate immune sensor cGAS. PLOS Pathogens, 2022. 18(7): p. e1010725.
62. Fehrmann, F. and L. A. Laimins, Human papillomavirus type 31 life cycle: methods for study using tissue culture models. Methods Mol Biol, 2005. 292: p. 317-30.
63. Longworth, M. S. and L. A. Laimins, The binding of histone deacetylases and the integrity of zinc finger-like motifs of the E7 protein are essential for the life cycle of human papillomavirus type 31. J Virol, 2004. 78(7): p. 3533-41.
64. Zagout, S., L.-L. Becker, and A. M. Kaindl, Immunofluorescence Staining of Paraffin Sections Step by Step. Frontiers in Neuroanatomy, 2020. 14.
65. Richard, P., S. Feng, and J. L. Manley, A SUMO-dependent interaction between Senataxin and the exosome, disrupted in the neurodegenerative disease AOA2, targets the exosome to sites of transcription-induced DNA damage. Genes Dev, 2013. 27(20): p. 2227-32.
66. Bennett, C. L. and A. R. La Spada, SUMOylated Senataxin functions in genome stability, RNA degradation, and stress granule disassembly, and is linked with inherited ataxia and motor neuron disease. Mol Genet Genomic Med, 2021. 9(12): p. e1745.
67. Liu, Z., et al., San1 deficiency leads to cardiomyopathy due to excessive R-loop-associated DNA damage and cardiomyocyte hypoplasia. Biochimica et Biophysica Acta (BBA)—Molecular Basis of Disease, 2021. 1867(11): p. 166237.

### Example 2: HPV Induced R-Loop Formation Represses Innate Immune Gene Expression while Activating DNA Damage Repair Pathways

### Introduction

R-loops are trimeric nucleic acid structures that are formed when an RNA strand hybridizes with its complementary DNA and displaces the opposite strand [1-5]. These structures are long-lived and regulate normal transcription as well as replication. Aberrant R-loops can form, and failure to efficiently resolve these structures leads to transcription/replication conflicts, resulting in DNA break formation [6-11]. High levels of R-loops have been detected in cell lines derived from precancerous lesions that maintain high risk human papillomaviruses (HPVs) [12-16]. Furthermore, human cancers themselves contain high levels of R-loops, which suggests they contribute to progression [17-20]. Few studies have, however, examined how R-loop distributions and functions differ between cells, such as those that maintain human papillomaviruses (HPVs) and normal cells. Our studies investigated how the landscape of R-loop distributions and functions change between cells that maintain high risk HPV-31 and normal keratinocytes.

HPVs are the etiological agents of cervical cancer and are responsible for ˜5% of all human cancers. Cancers and precancers induced by infection with high-risk HPVs provide an excellent model for studying factors influencing progression [21-23]. Cervical lesions caused by high-risk HPVs are characterized as cervical intraepithelial neoplasia grades I to III (CINI-CINIII), and these precede the development of frank cervical cancer [24-26]. Characterization of lesions as CIN I to CINII is made according to the degrees to which epithelia are altered. In precancerous CIN I lesions, HPV genomes are maintained as extrachromosomal elements or episomes that replicate coordinately with cellular replication, while productive viral replication or amplification is restricted to differentiated suprabasal cells [27-29]. CIN 612 is an immortal cell line that was derived from a CINI cervical biopsy and stably maintains high-risk HPV 31 genomes as episomes [30]. Transfection of normal human keratinocytes with cloned HPV sequences leads to their immortalization and stable maintenance of viral episomes [31]. These cell lines are similar to those derived from CIN I lesions and demonstrate that viral genomes are responsible for changes indicative of precancerous lesions. Previous studies demonstrated the presence of high levels of R-loops in CIN612 cells in comparison to normal human keratinocytes (HFKs) [20, 32]. Furthermore, R-loops formed on HPV genomes as well as cellular sites, and these high levels were found to be critical for viral transcription as well as replication. Elevated levels of R-loops have also been detected by immunofluorescence and immunohistochemistry analyses of biopsies from HPV positive cervical cancers [20, 33]. In this study, we examined how the landscape of R-loops on cellular sites changes between HPV positive cells and normal keratinocytes as well as whether these alterations have functional consequences on cellular gene expression and HPV pathogenesis.

### Results

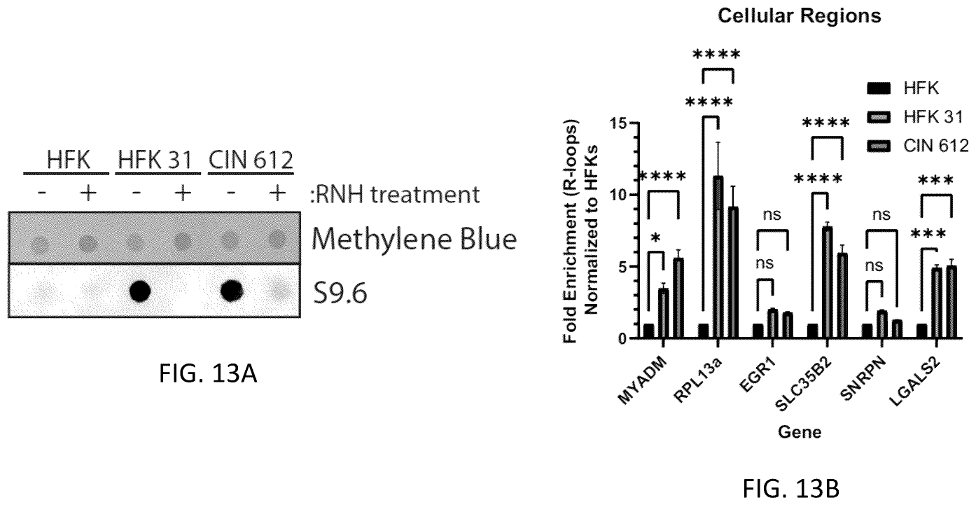

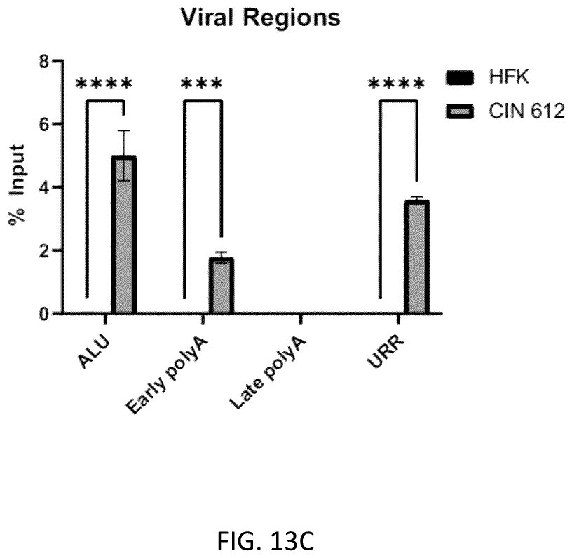

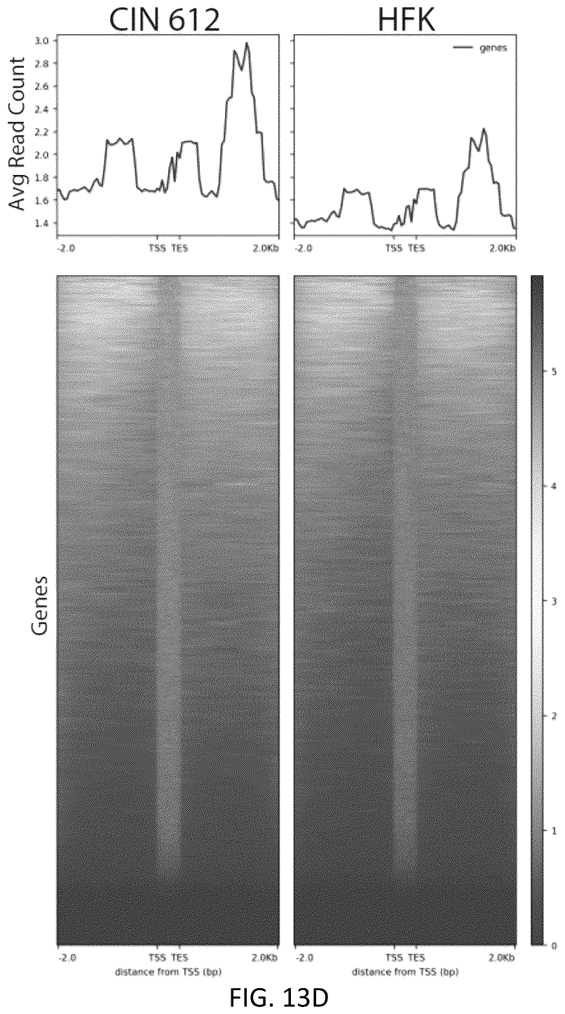

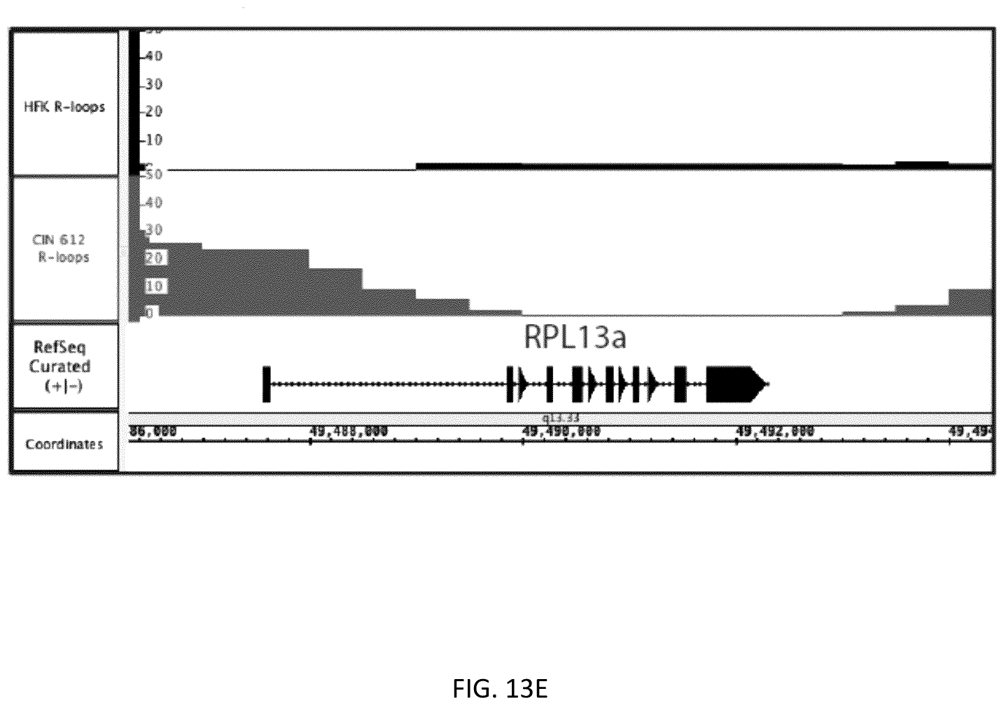

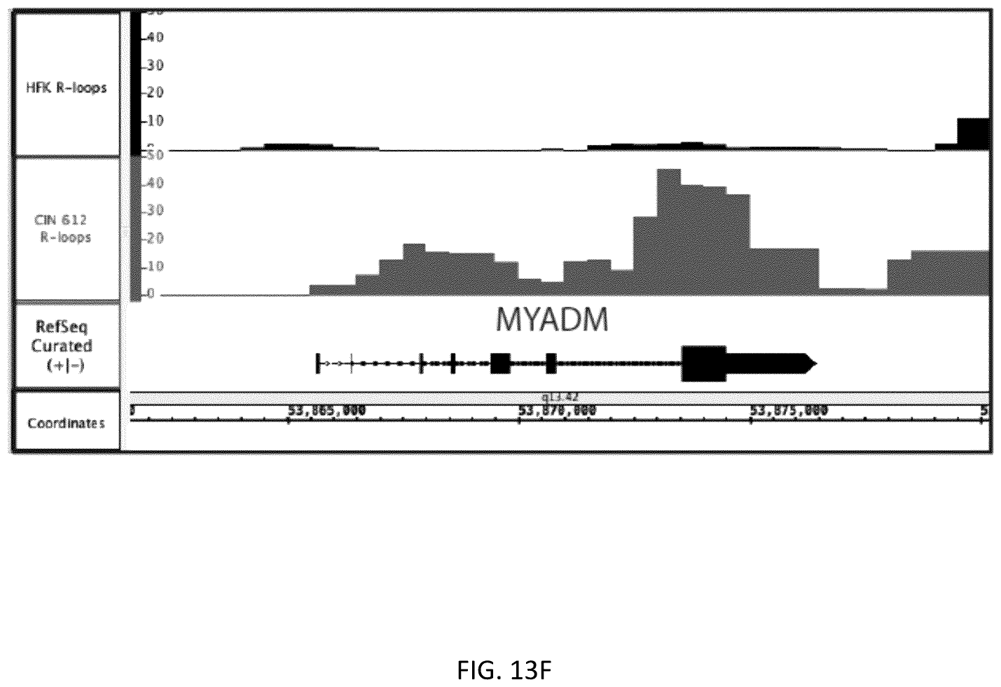

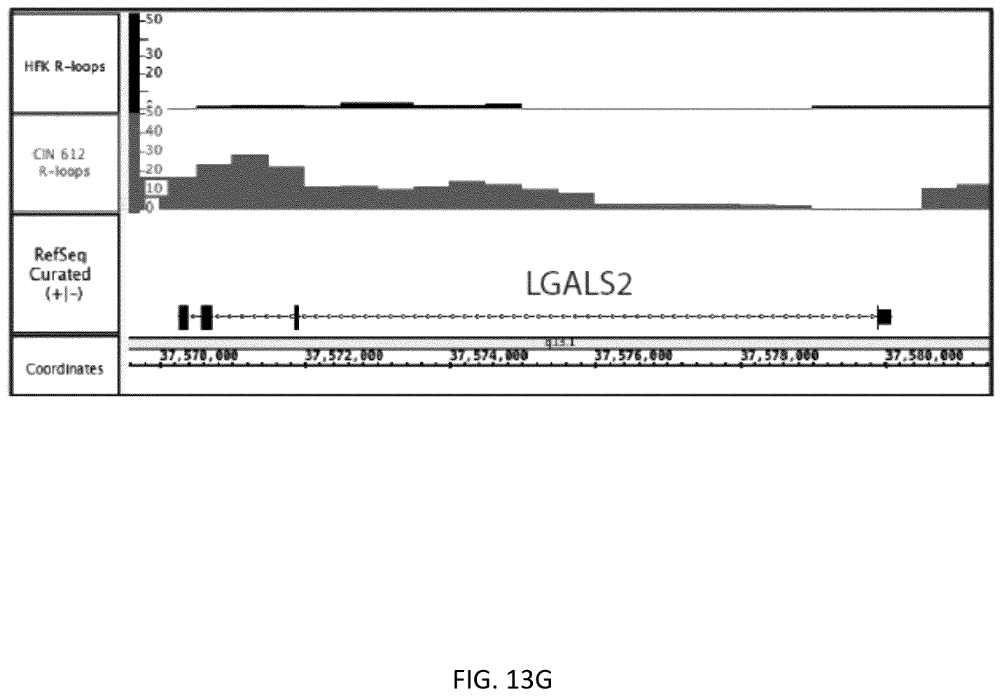

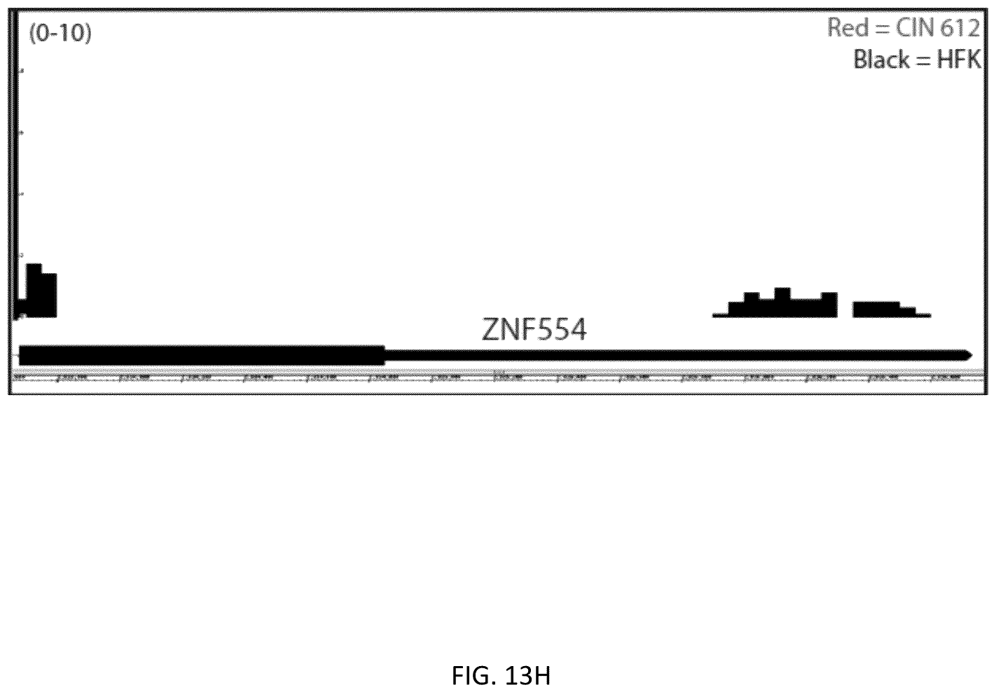

To investigate how the distributions and functions of R-loops change due to the presence of high-risk HPV genomes, we examined cells derived from a biopsy of an HPV 31 positive precancerous cervical lesion (CIN 612) and compared effects in normal keratinocytes (HFKs). Included in this initial analysis was the HFK-31 cell line that was generated by transfection of HFKs with cloned HPV 31 sequences and maintains viral sequences as episomes. Both HPV positive cell lines have been shown to exhibit similar histological changes in organotypic raft cultures consistent with CIN I lesions in vivo [31, 34]. The levels of total R-loops in these cells were measured by dot blot assays that utilize the S9.6 antibody, which is specific for R-loops (FIG. 13A). This analysis demonstrated that total R-loop levels in both HPV positive cell lines were significantly increased compared to normal keratinocytes. RNase H treatment abrogated these signals, demonstrating the assay was specific for R-loop formation. R-loops often form at promoter as well as transcription termination sequences, and the examination of these regions on a series of representative genes by DRIP-qPCR demonstrated substantially increased levels in HFK-31 and CIN 612 cells (FIG. 13B). In this analysis, the levels of R-loops at previously reported sites in MYADM, RPL13a, SLC35B2, and LGAL2 were found to be increased on average by 5-to-10-fold relative to levels detected at the same sites in normal keratinocytes [12, 16, 35-37]. In addition, sites with low or negligible levels of R-loops associated with genes such as EGR1 and SNRPN in normal keratinocytes showed minimal increases in CIN 612 cells (FIG. 13B). Increases of over 500-fold in R-loop levels were also detected in association with ALU elements, which may account for the higher total levels seen by dot blot analysis (FIG. 13C). High levels of R-loops were also detected on HPV genomes at the early promoter and termination sites but not at coding sequences or the late poly A site (FIG. 13C). These observations indicate there are substantial increases in R-loop levels in cells derived from HPV positive precancers or generated by transfection in comparison to normal keratinocytes and are consistent with previous reports [20].

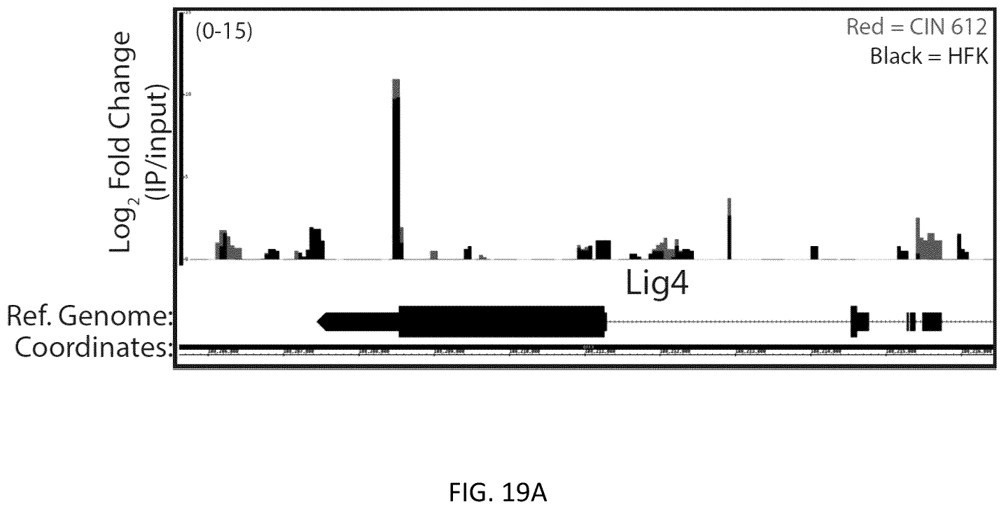

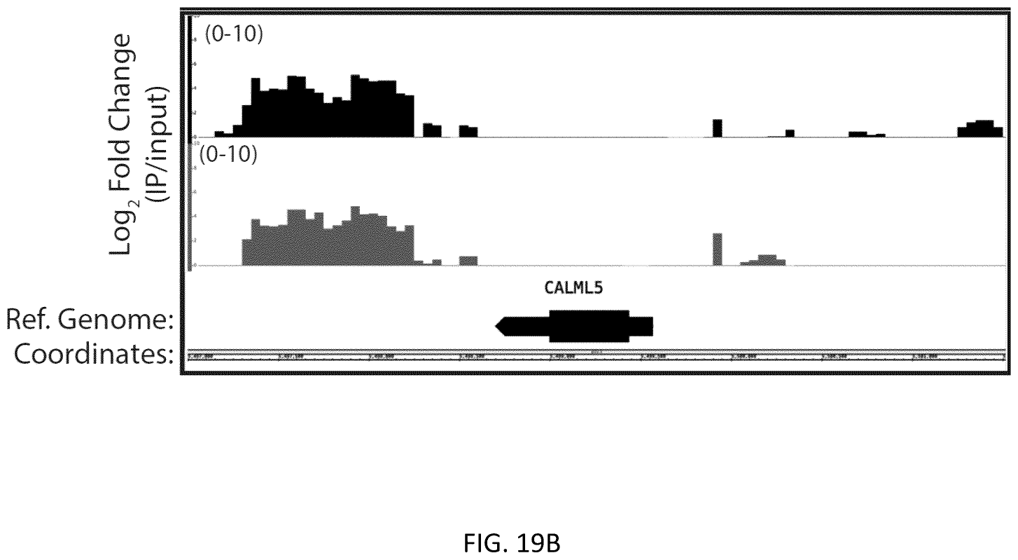

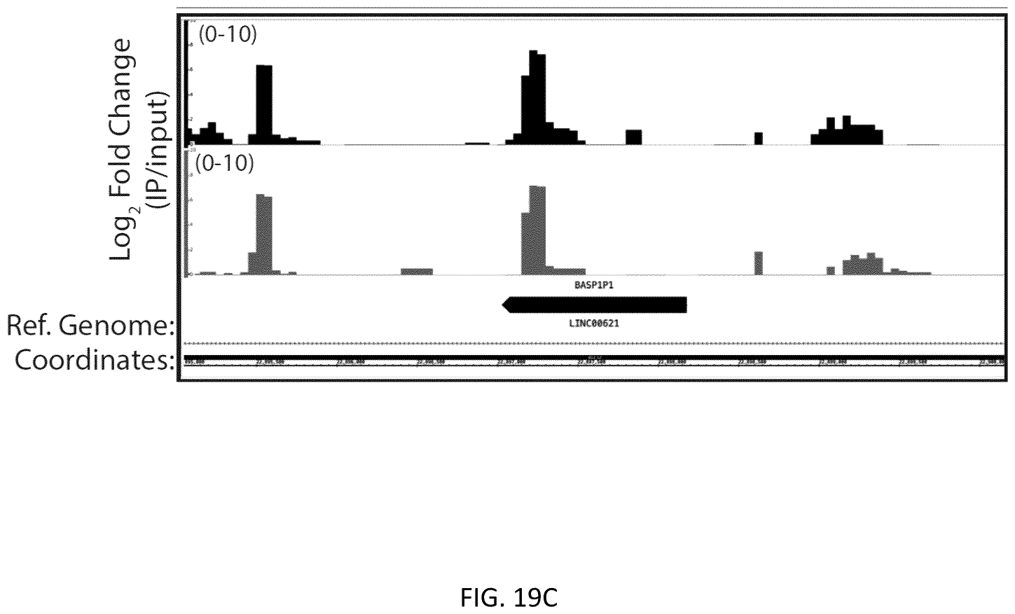

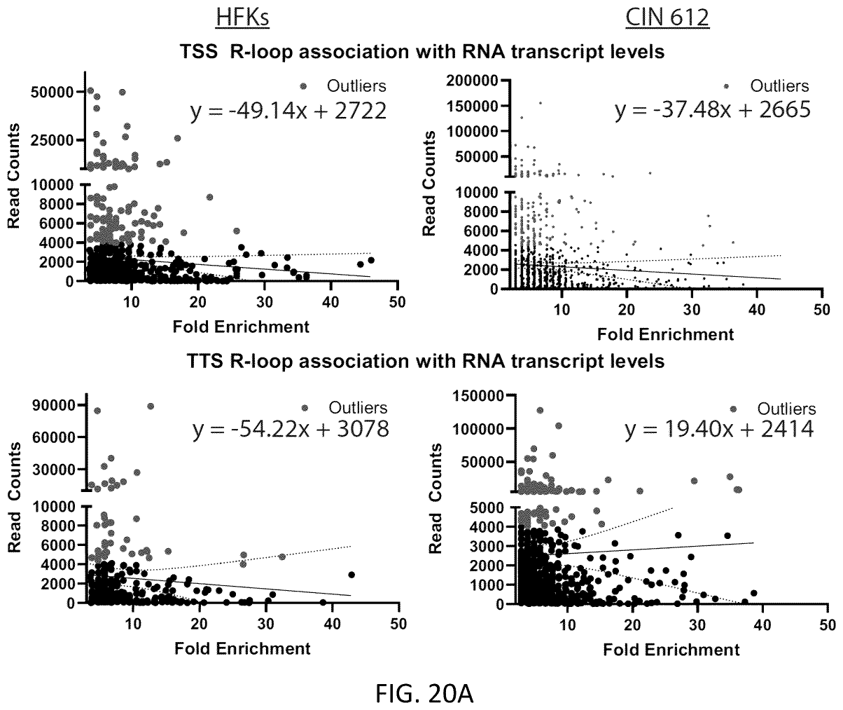

DRIP-sequencing (DRIP-seq) was next performed to investigate how the distributions of R-loops varied between HPV positive cells and normal keratinocytes. This method allows for an unbiased approach to identify where R-loops are present within cells utilizing immunoprecipitations with the S9.6 monoclonal antibody followed by NEXTGen sequencing [38]. We focused this analysis on CIN612 cells in comparison to HFKs. Metaplot analysis of R-loop distribution of 2 kb upstream and downstream of coding sequences in normal keratinocytes (HFK) and CIN 612 cells demonstrated that R-loop reads in both cell types peak near the transcription start site (TSS), at the transcription end site (TES), and about 1-1.5 kb downstream of the TES. This distribution is similar to the profile published by Promonet et al. [12]. For our downstream analyses, R-loops within these regions were associated with a gene's coding region and referred to as genic R-loops. Importantly, the overall R-loop distributions at TSS and TES sites are similar in CIN 612 cells (FIG. 13D, left) as well as in normal keratinocytes (FIG. 13D, right). The primary difference between the two cell types was that the signal in the CIN 612 cells was significantly higher than in the normal keratinocytes. Heatmap analysis demonstrated that genes with high levels of R-loops at the TSS also had high levels of R-loops at the TES (FIG. 13D, bottom). Only a minority of genes exhibited distinct patterns of R-loop formation in CIN 612 compared to normal keratinocytes. Examples of R-loops distributions showing the IP/input enrichment on four different genes are shown in FIGS. 13E-H. RPL13a, MYADM, and LGALS2 all contained significantly higher R-loops levels over the input background control in the precancerous CIN 612 cells than in normal keratinocytes, consistent with our DRIP-qPCR analysis (FIGS. 13B-13H). ZNF554 is a gene not associated with R-loop formation, and it exhibited minimal R-loop levels over the input background in both cell lines examined. FIGS. 19A-C show similar analyses for 2 additional genes (DNA Lig IV and CALML5). This overview indicates that enhanced levels of R-loops are present in CIN 612 cells compared to normal keratinocytes, and these form at similar, though not necessarily identical, regions in the proximal 2 kb upstream and downstream regions.

Identification of Genomic Regions Enriched with R-Loops

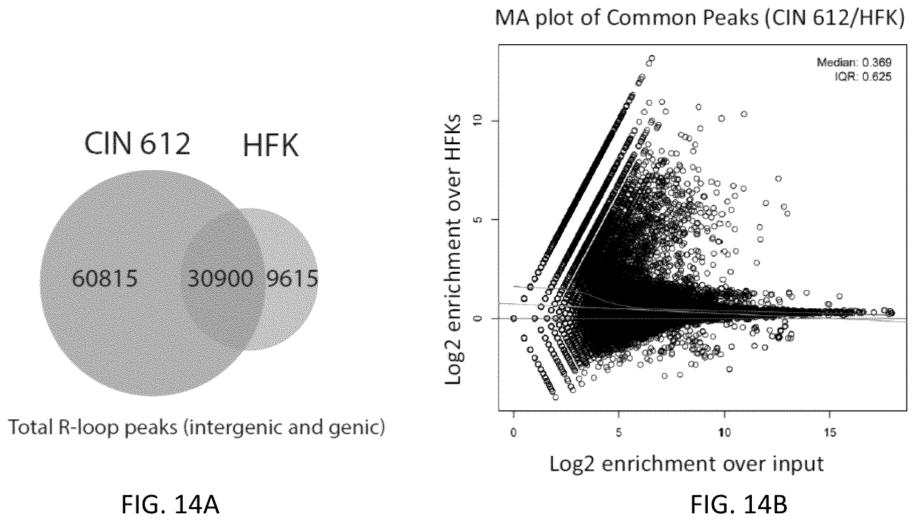

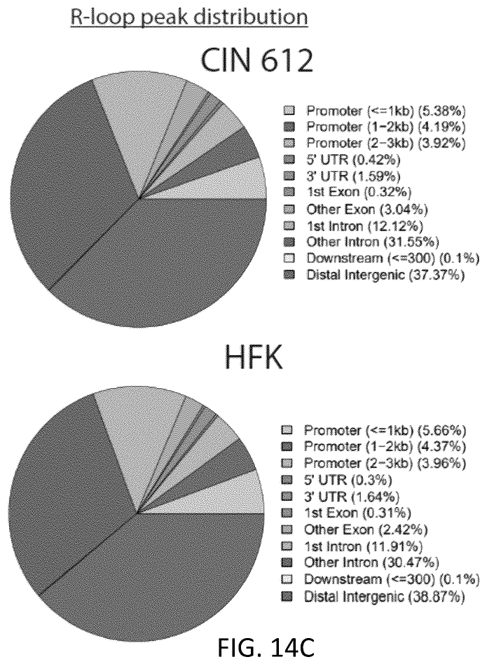

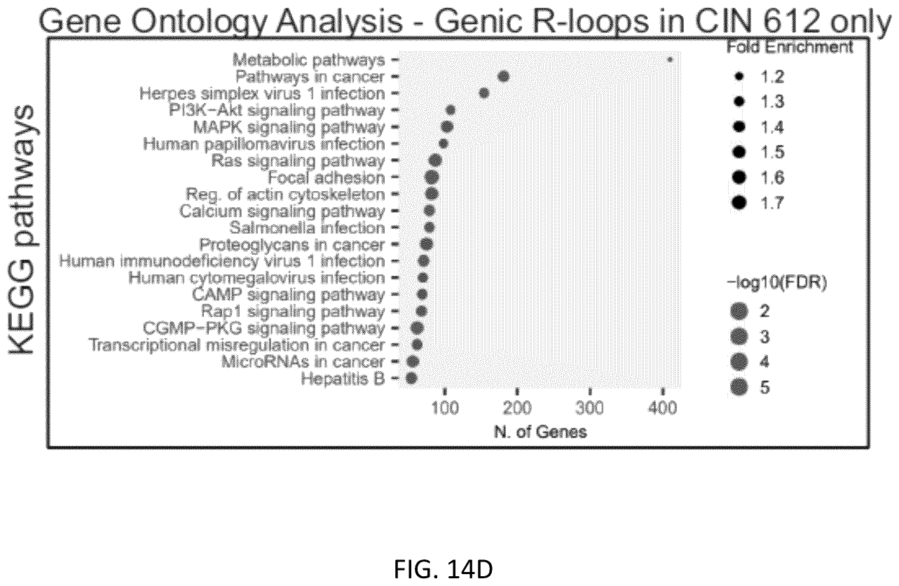

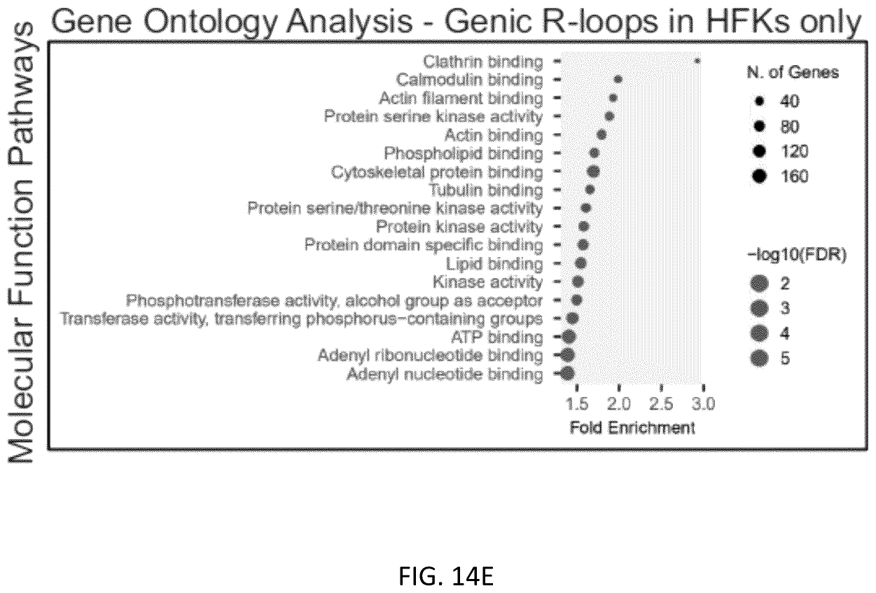

Further analysis of the DRIP-seq data was then used to provide an overall picture of which sites were associated with enhanced R-loop formation in CIN 612 cells as compared to normal keratinocytes. Peak calling analysis demonstrated that R-loops were significantly enriched over background at over 90,000 sites in HPV positive cells and at approximately 40,000 sites in normal keratinocytes. About 30,000 sites were shared between both cell lines, leaving over 60,000 unique R-loop sites in CIN 612 cells and approximately 9,500 unique sites in normal keratinocytes (FIG. 14A). MA plot analysis of the ˜30,000 common sites indicated significantly higher R-loop levels at those sites in CIN 612 cells compared to normal keratinocytes, with some R-loop sites being enriched by almost ˜1000 fold in the HPV positive cells (FIG. 14B). Although there are significant differences in the numbers of R-loop reads between CIN 612 cells and normal keratinocytes, peak distribution over promoter, exonic, and downstream sequences were very similar, with approximately 60% of the R-loop peaks detected in genic regions in both cell types (FIG. 14C).

Since the levels of R-loops were substantially increased in HPV positive cells relative to normal keratinocytes, it was possible they were linked to specific genes or pathways. For this analysis, we first examined genes associated with R-loops in both normal keratinocytes and CIN 612 cells, focusing on proximal promoter, gene body, and terminator regions. Pathway analysis on the R-loop containing genes was then performed using ShinyGO 0.80 [39]. The KEGG pathways associated with high levels of R-loops in CIN 612 cells included those involving cancer progression (pathways in cancer, proteoglycans in cancer, and transcriptional misregulation in cancer) and DNA virus infection, many of which were not found to be enhanced in normal keratinocytes (FIG. 14D). In contrast, pathway analysis of genes containing R-loops in normal keratinocytes identified prominent pathways involved in clathrin binding, lipid binding, and kinase activity (FIG. 14E). These analyses indicated that the increased formation of R-loops in CIN 612 cells is localized to genes in pathways that are distinct from those seen in normal keratinocytes. It was next important to determine if this increased R-loop association correlated with increased transcription.

HPV Positive Cells have Similar Numbers of Genes Upregulated and Downregulated, Despite High R-Loop Levels

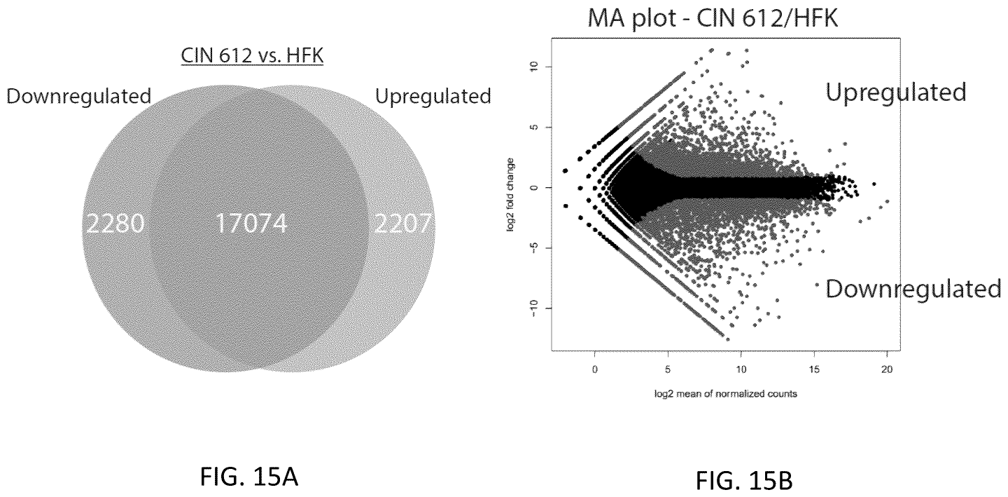

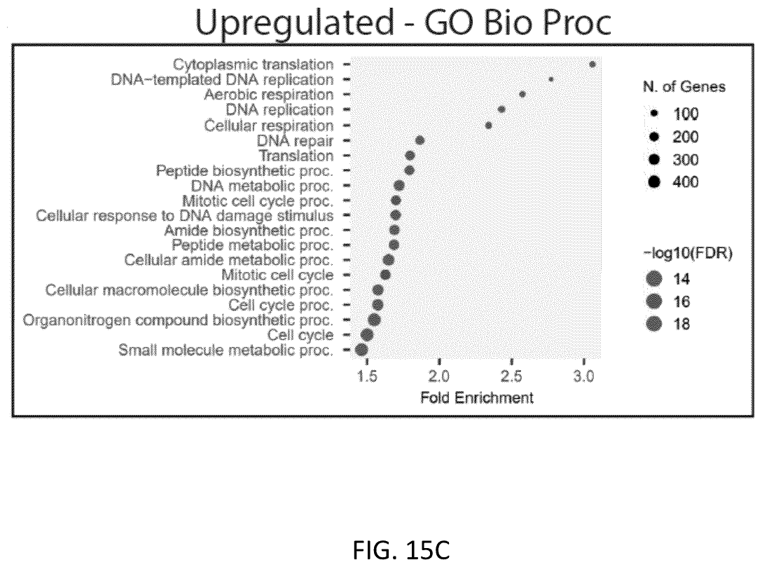

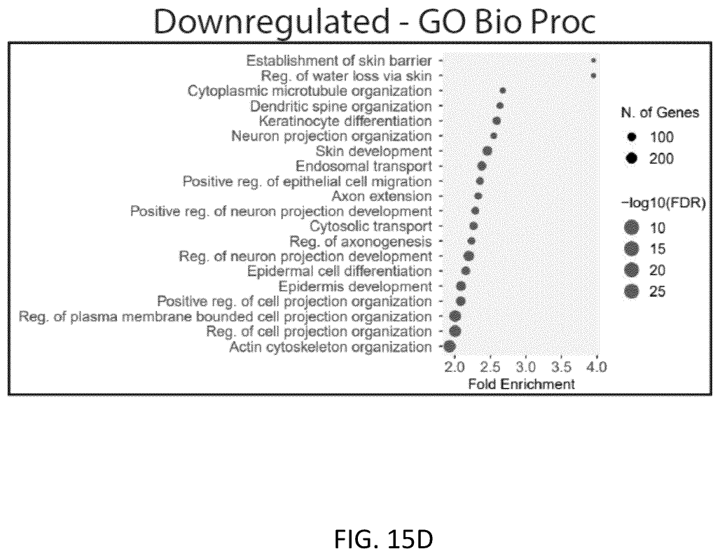

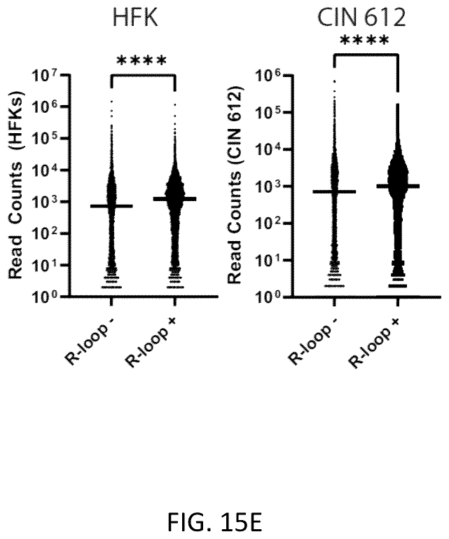

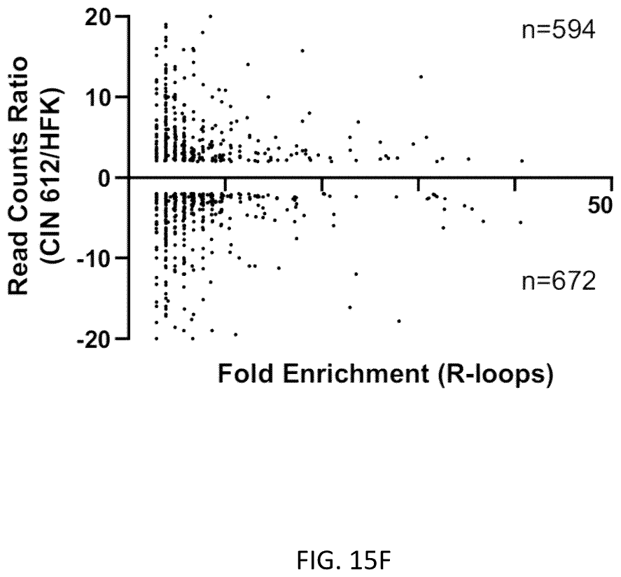

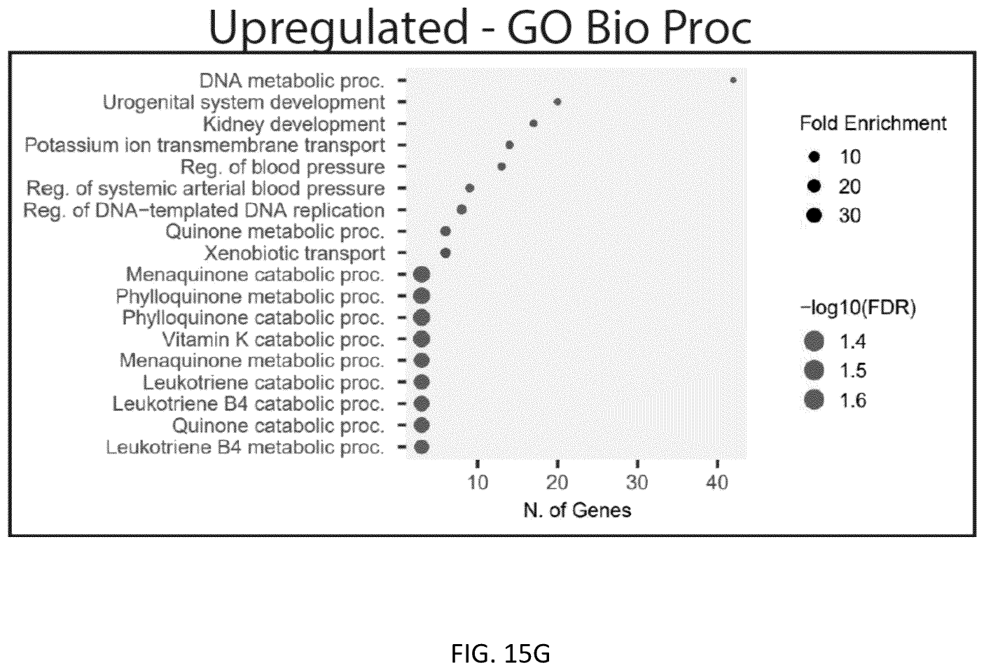

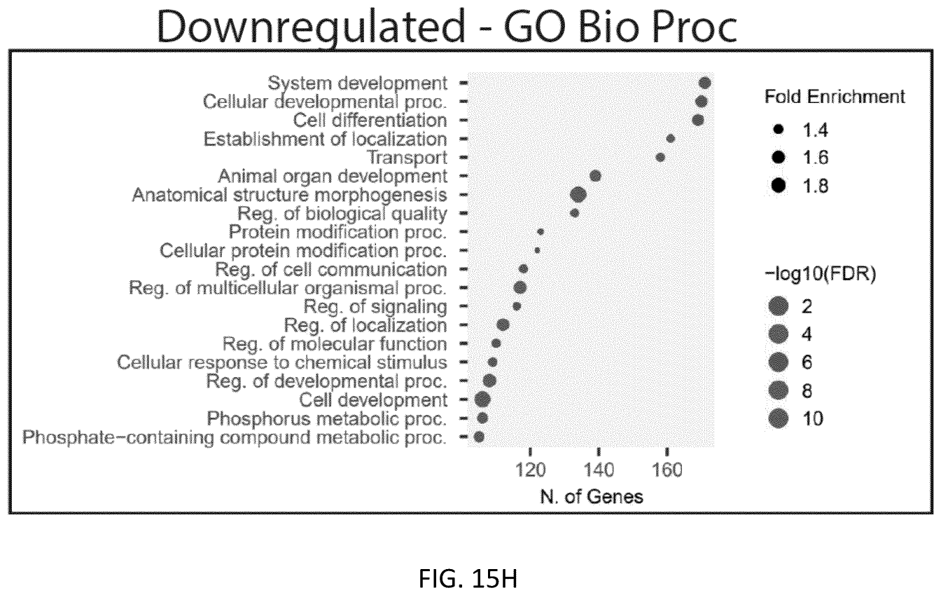

In order to determine if there was a correlation between high levels of R-loops and increased transcription of the associated genes, RNA sequencing was performed on CIN 612 [20] and normal keratinocytes. This analysis demonstrated that approximately 20% (˜4,500) of the genes analyzed were differentially expressed compared to normal cells. Interestingly, these genes were divided almost equally between those upregulated (2,207) and those downregulated (2,280) (FIG. 15A). A similar distribution in the fold changes of differentially expressed genes was seen by MA plot analysis (FIG. 15B). We then performed pathway analysis of the differentially expressed genes in CIN 612 cells compared to normal keratinocytes to determine which pathways were altered (FIGS. 15C-D). Consistent with previous findings, genes in pathways involved in DNA repair, DNA replication, and cell cycle were upregulated, while those in pathways important for epithelial differentiation and epidermal development were downregulated [40, 41]. In addition, pathways involved in the immune response and interferon signaling were downregulated in CIN 612 cells.

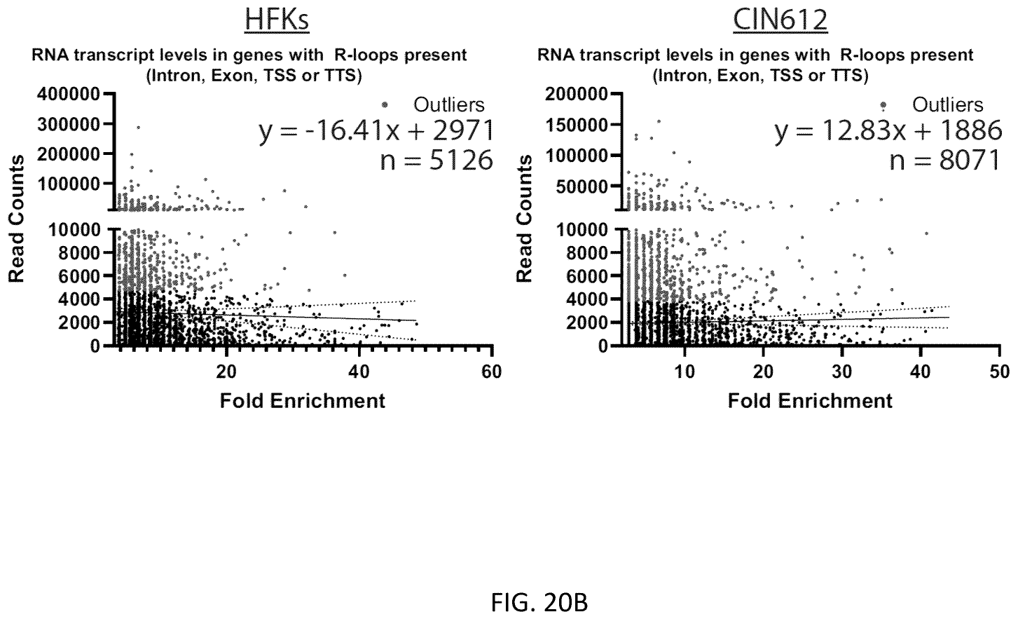

DRIP-seq analyses were then used to investigate if altered expression levels were linked to increased R-loop levels. This analysis demonstrated an approximately 2-fold higher level of total transcripts associated with genes that were linked to R-loops compared to those without R-loops (FIG. 15E). Importantly, approximately 30% of the differentially expressed genes were found to be associated with R-loops only in CIN 612 cells and absent in normal keratinocytes (FIG. 15F). These differentially regulated genes were similarly distributed between those downregulated (672) and those upregulated (594). While there was not a consistent global increase or decrease in the expression of genes that were uniquely associated with R-loops in CIN 612 cells, KEGG analysis identified genes in specific pathways that were coordinately regulated (FIGS. 15G-H). The most prominent pathways associated with enhanced R-loop levels were involved in replication and DNA metabolism. At the same time, genes associated with the innate immune surveillance pathway, including IL1B, STAT1, and MYD88, were linked to enhanced R-loop levels but exhibited decreased expression in CIN 612 cells relative to normal keratinocytes. Furthermore, no correlation was found between the enrichment of R-loops and their corresponding mRNA levels with respect to whether these structures formed at either TSS, TES, or intronic locations (FIGS. 20A-B).

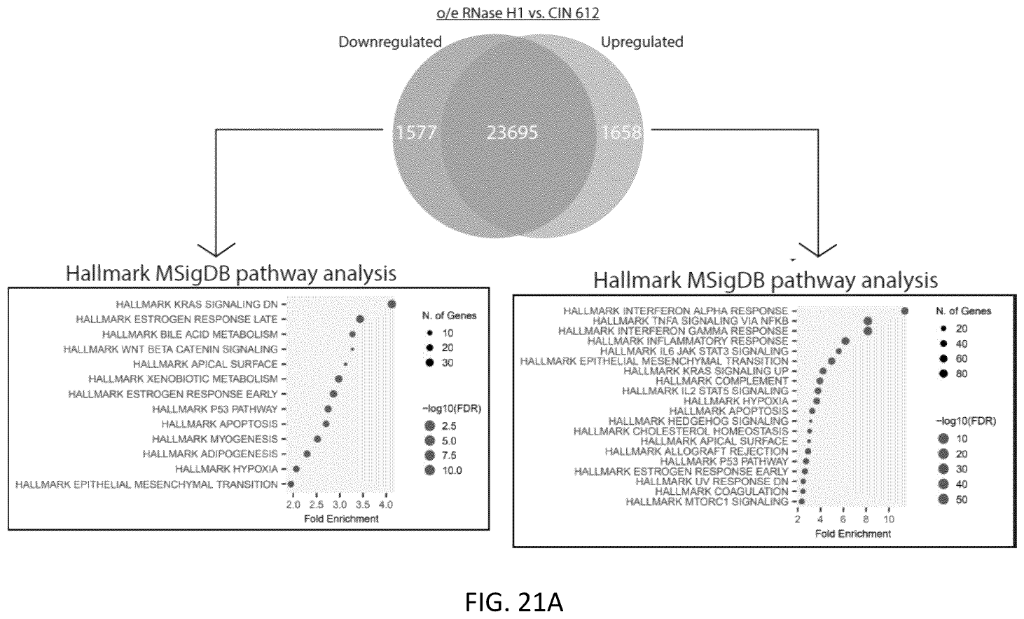

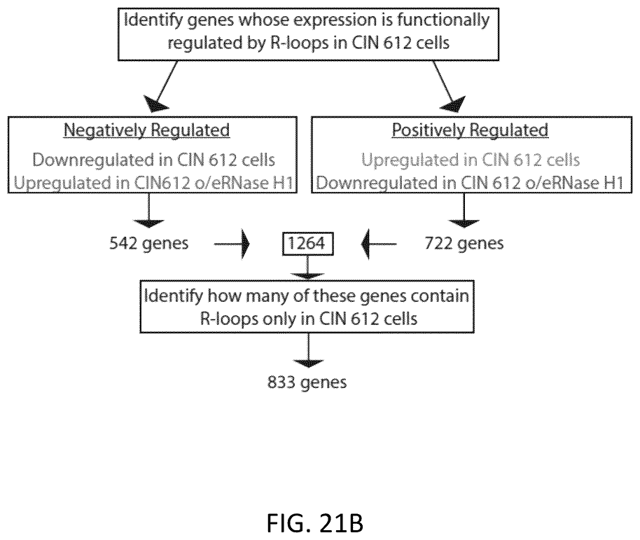

The above studies indicated there was a correlation between the presence of unique R-loops in CIN 612 cells and altered expression of genes in specific pathways. It was next important to determine if their expression was functionally dependent upon enhanced levels of R-loops. For this analysis, we utilized CIN 612 cells that were generated to overexpress RNase H1, an R-loop processing enzyme, by transfection of CMV-directed tagged expression vector followed by selecting stable cell lines [42]. Overexpression of RNAse H1 has been characterized as the “gold standard method” for reducing R-loop levels [43]. When RNase H1 was overexpressed in CIN 612 cells, viral early gene expression and episome levels were reduced by ˜70% and 50%, respectively. As HPV 31 E6 was shown to induce R-loop formation in HFKs, this could further enhance these reductions [20]. RNA-seq analysis was previously performed on these cells and compared to that seen with parental CIN 612 cells [20]. The cells overexpressing RNase H1 exhibited substantially reduced levels of R-loops compared to the parental line. Around 12% of all genes (FKPM>0) were differentially expressed within CIN 612 cells overexpressing RNase H1 compared to the scramble control cells (FIG. 21A). Furthermore, 833 of the genes were associated with R-loops only in CIN 612 cells, further supporting a direct functional relationship between R-loop formation and gene expression (FIG. 21).

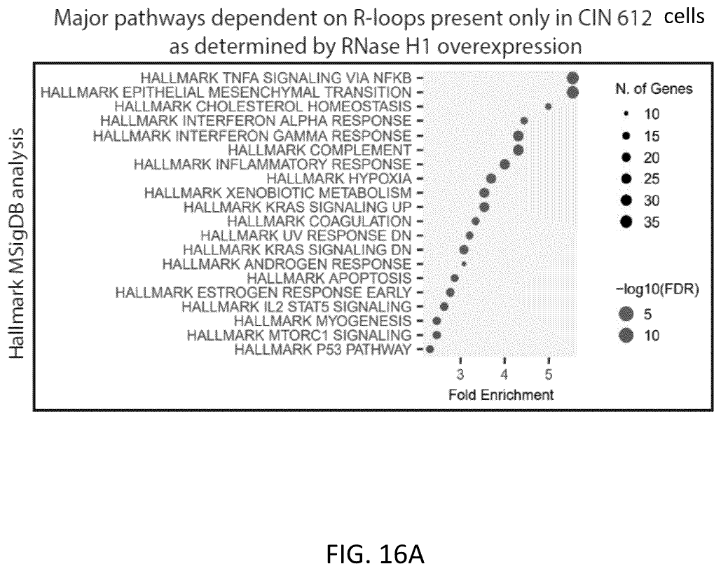

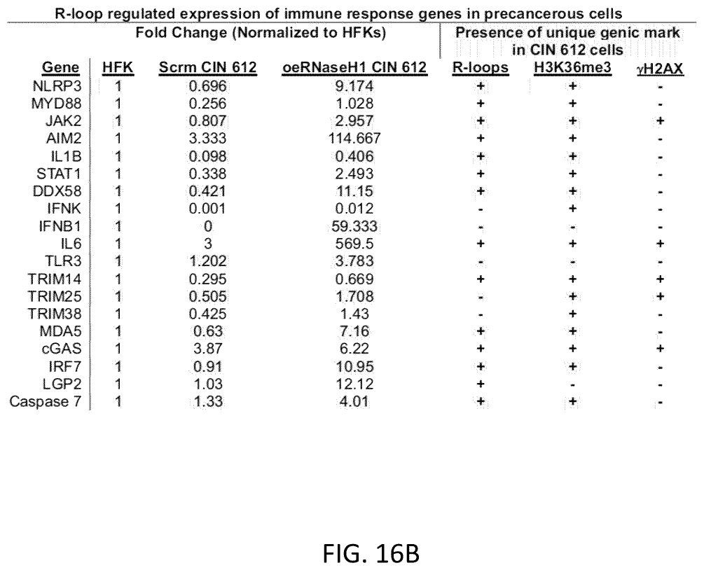

Pathway analysis of genes whose expression was dependent upon R-loops present only in CIN 612 cells were linked to innate immune surveillance, including interferon-alpha and interferon-gamma responses, complement signaling, and inflammation (FIG. 16A). Genes in these innate immune surveillance pathways that are repressed by R-loops in CIN 612 cells include STAT1 (8-fold), NLRP3 (14-fold), JAK2 (4-fold), AIM2 (34-fold), RIG-I (22-fold), IFN β (greater than 50-fold), and IL6 (190-fold). In contrast, TRIM 14 and STING are only modestly repressed (FIG. 16B). The expression of some genes, such as IFN β and RIG-I, increased in response to reductions in levels of R-loops despite not being physically linked with these structures. This likely indicates that R-loops target their upstream regulators, so it is likely that the effects on IFN β and RIG-I are indirect.

Histone Modifications are Differentially Deposited on Host Chromatin within HPV Positive Cells

The linkage of enhanced levels of R-loops with coordinated expression of genes in multiple specific pathways suggested that additional factors act to facilitate this specificity. One way R-loops could coordinate the expression of genes in distinct pathways might be through association with different sets of modified histones that configure chromatin around these structures [44]. In addition to histones linked to chromatin states, R-loops are also associated with the modified histone, γH2AX, which is coupled with DNA break formation and may be linked to gene expression [12, 45, 46]. Therefore, we investigated whether there are associations between specific sets of histones and R-loop-directed gene expression that vary between normal and HPV-positive cells.

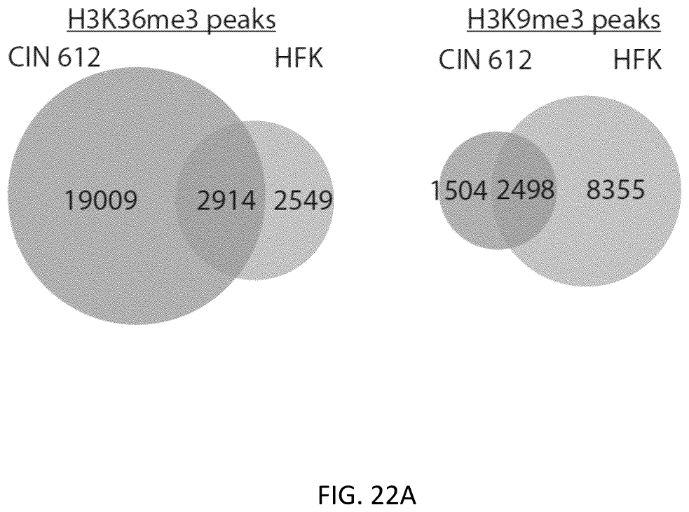

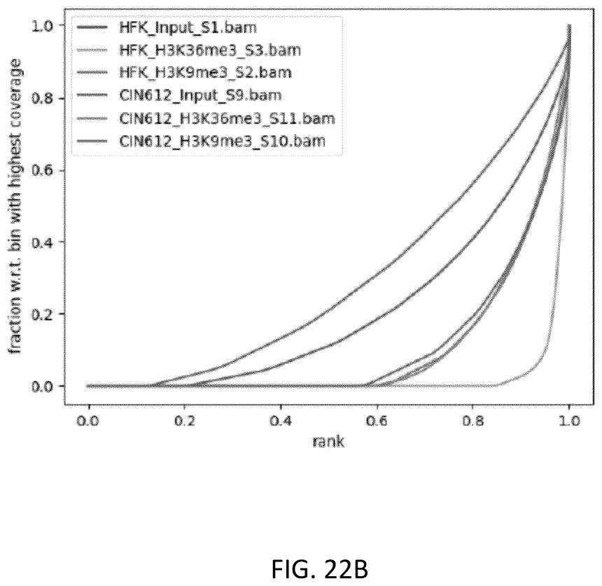

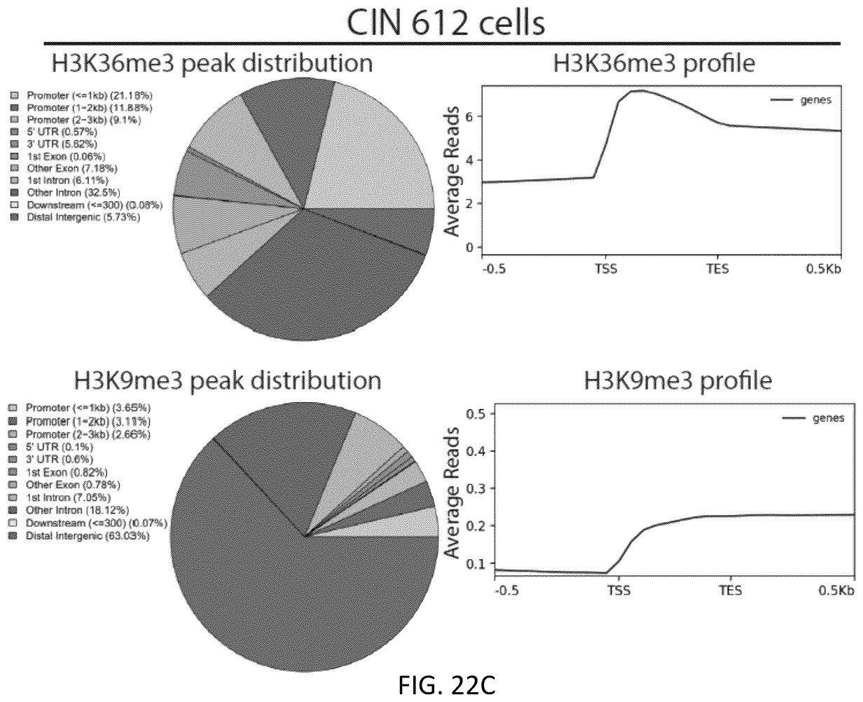

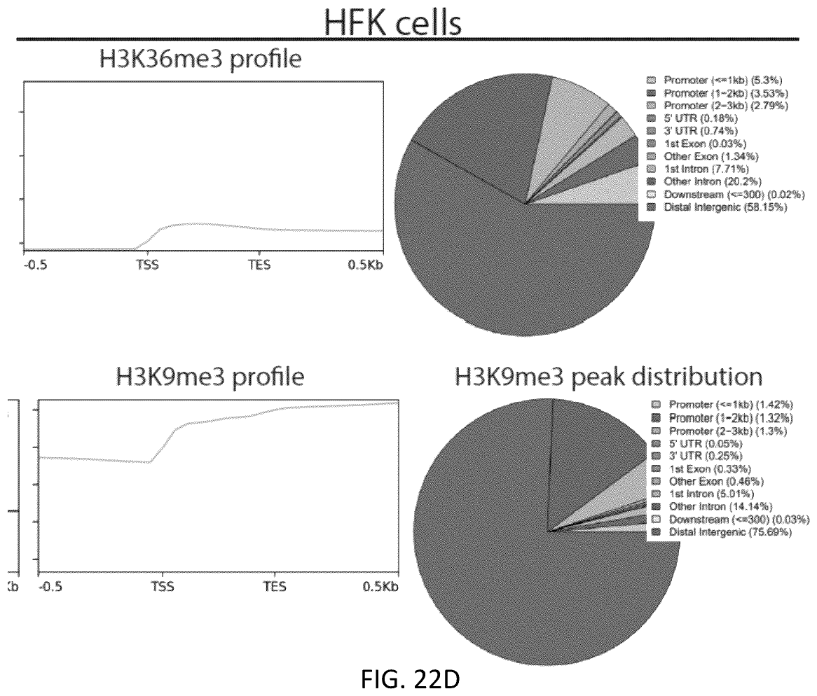

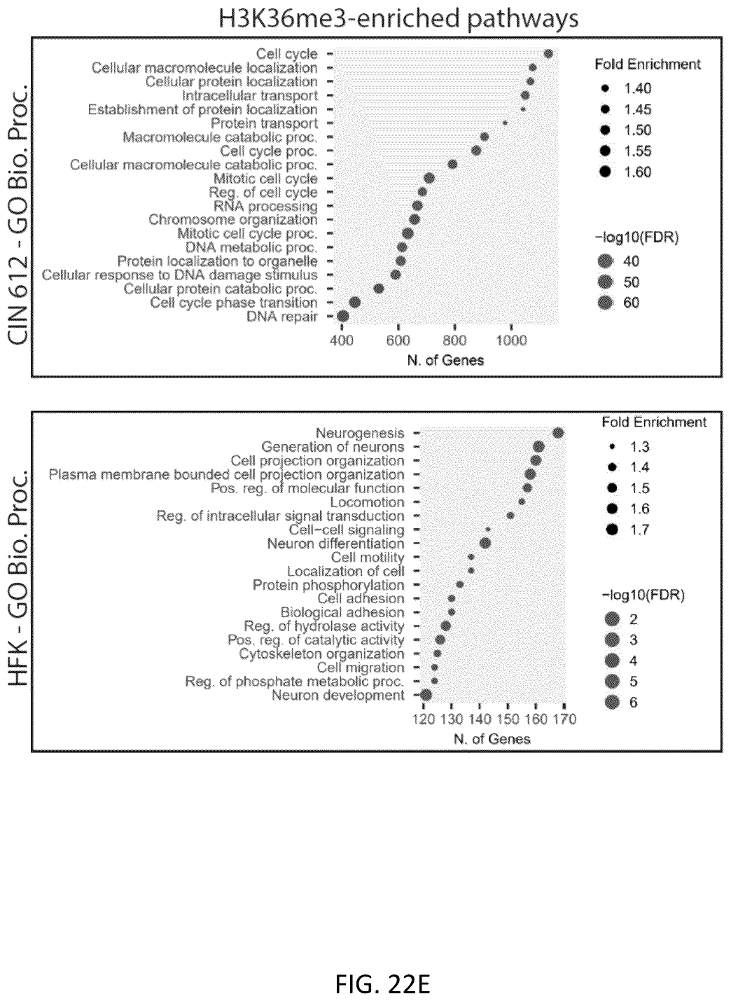

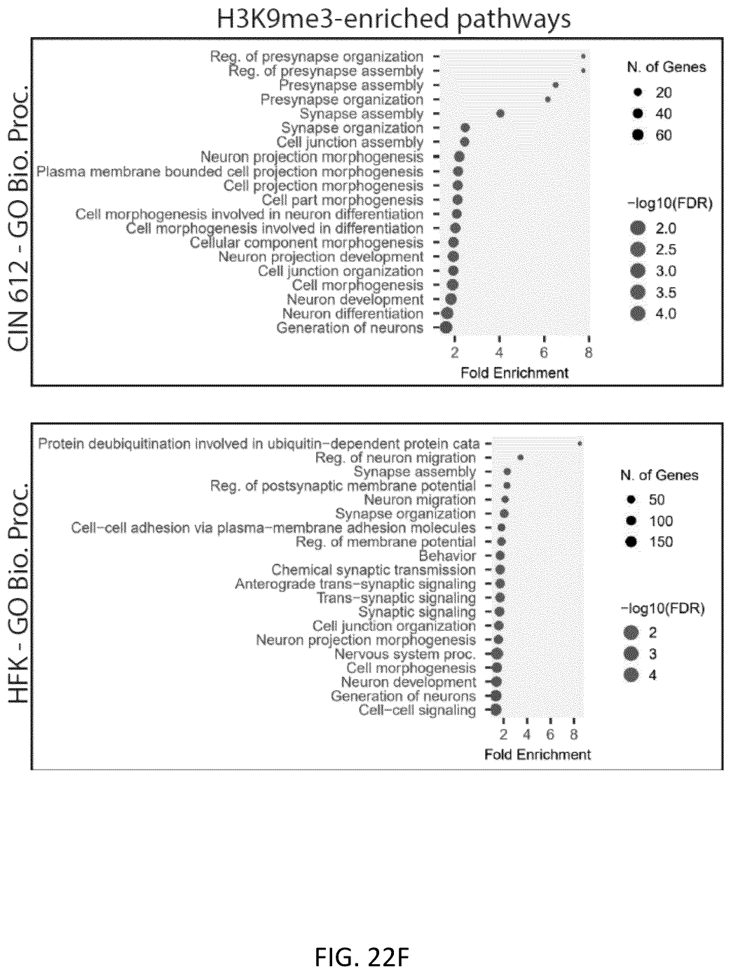

For this analysis, chromatin immunoprecipitation was performed on CIN 612 cells and normal keratinocytes for three histone marks: H3K36me3, H3K9me3, and γH2AX. H3K36me3 is typically associated with transcription, while H3K9me3 marks areas of heterochromatin [47-50]. We performed peak calling algorithms on each of the modified histones pulldown experiments using the MACS peak calling algorithm. We controlled for off-target pulldowns by background subtracting respective input control samples isolated from each of the cell lines (HFK and CIN 612 cells). The called peaks were then assigned a relative genomic location using HOMER. Peak calling analysis for these histone marks focused on the regions 2 kb upstream, 2 kb downstream, or in the gene body in both CIN 612 and normal keratinocytes. This analysis identified an overlap of these histones with unique and common sets of genes associated with R-loops. Overall, H3K36me3 marks were approximately 4-fold more prevalent in CIN 612 cells than in normal cells (FIGS. 22A-F). In contrast, the opposite was found for H3K9me3 marks which were reduced in CIN 612 cells relative to normal keratinocytes (FIGS. 22A-F).

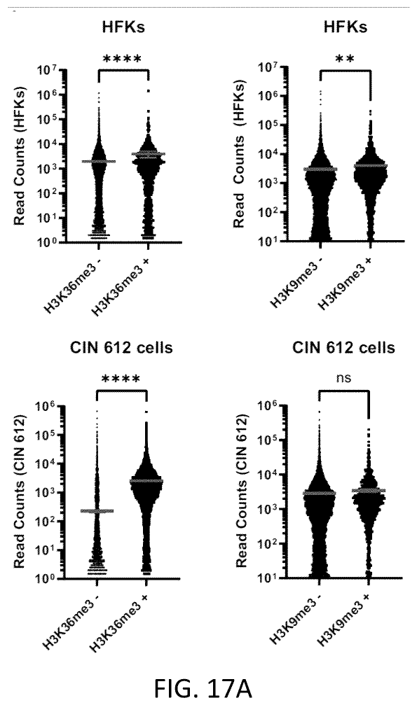

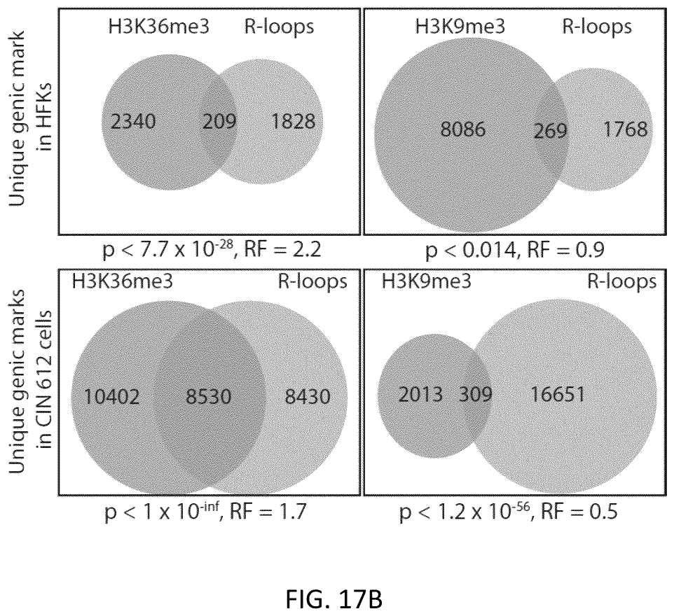

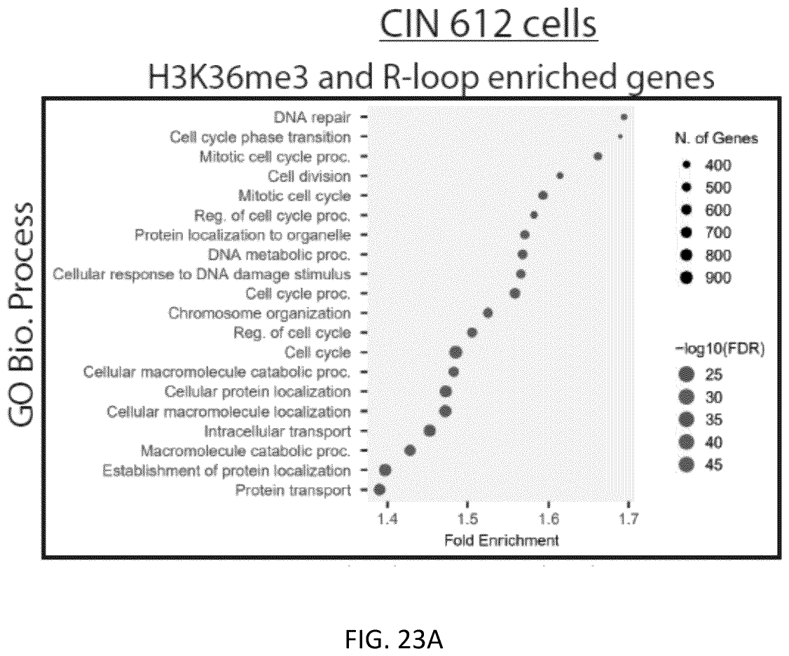

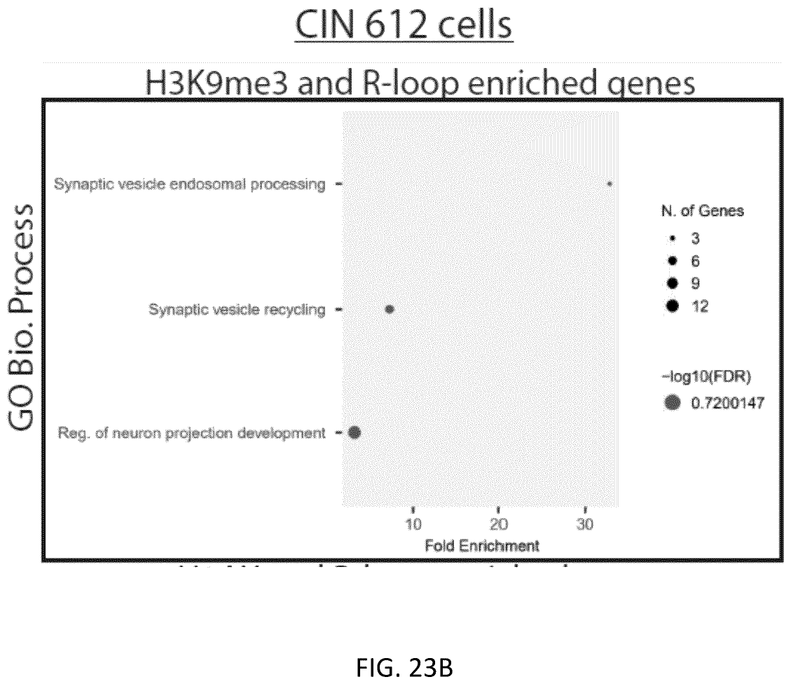

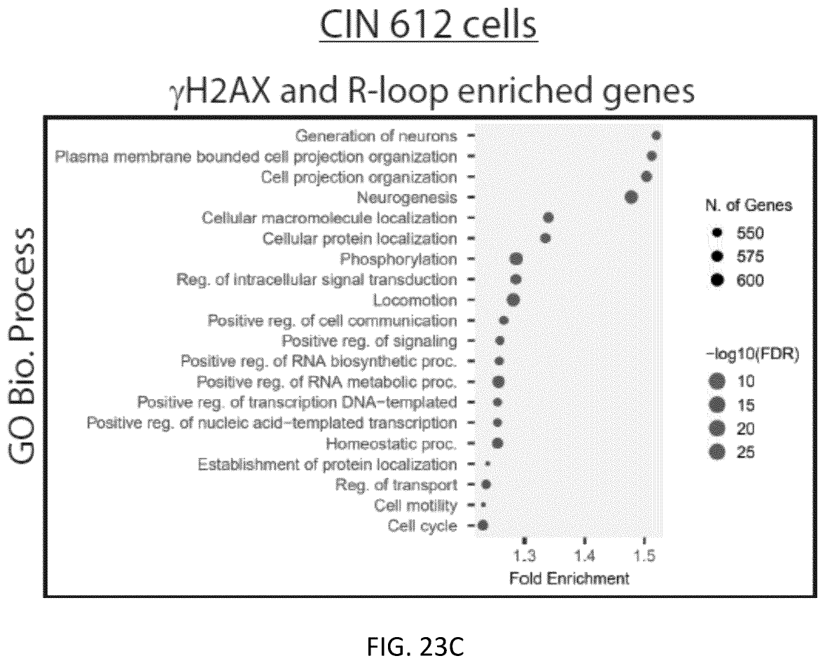

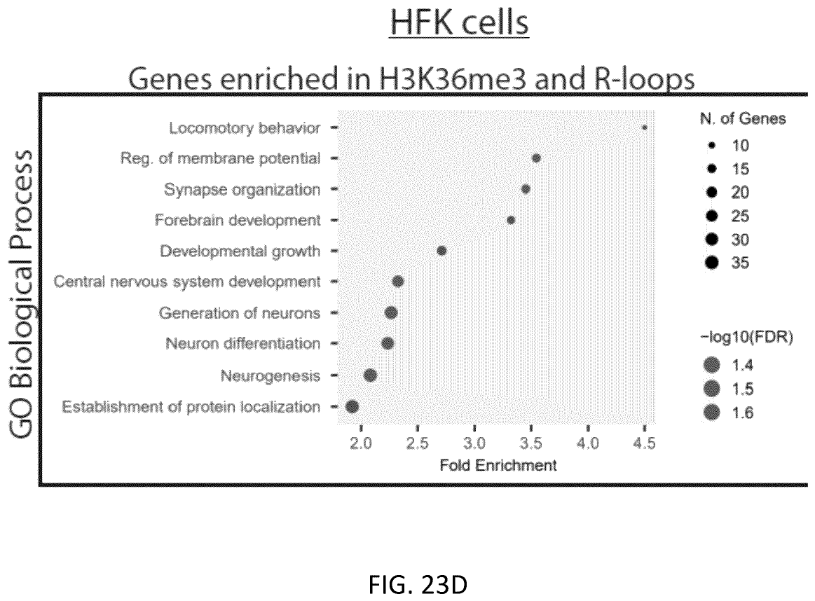

It was next important to determine whether the presence of H3K36me3 and H3K9me3 formation correlated with the enhanced formation of R-loops and transcription in HPV positive cells by comparing ChIP-seq analysis for these histones to DRIP-seq and RNA-seq data, respectively. RNA-seq analysis of genes containing H3K36me3 marks identified an over 2-fold enrichment in mRNA levels of these genes over those that did not contain H3K36me3 in both normal and CIN 612 cells (FIG. 17A, left). In contrast, the H3K9me3 mark on genes that were linked to R-loops in normal keratinocytes correlated with a modest 0.33-fold increase in mRNA levels, with no significant difference seen in the CIN 612 cells (FIG. 17A, right). Comparing the ChIP-seq analyses with DRIP-seq, over 50% of the R-loop containing genes in CIN 612 cells were also positive for H3K36me3 (FIG. 17B, bottom left), while less than 8% were associated in normal keratinocytes (FIG. 17B, top left). In contrast, less than 2% of genes with R-loops in CIN 612 cells were H3K9me3 positive as compared to over 10% in normal keratinocytes (FIG. 17B, right). Gene ontology analysis of the H3K36me3 and R-loop positive genes in CIN 612 cells identified pathways associated with cell cycle progression and innate immune surveillance (FIG. 23A). Genes in the immune response pathways identified above exhibited a strong linkage between the presence of both R-loops and H3K36me3 (FIG. 16B). In contrast, in normal keratinocytes, genes associated with H3K36me3 and R-loops were found in distinctly different pathways that regulate membrane potential, protein localization, and neurogenesis (FIG. 23D).

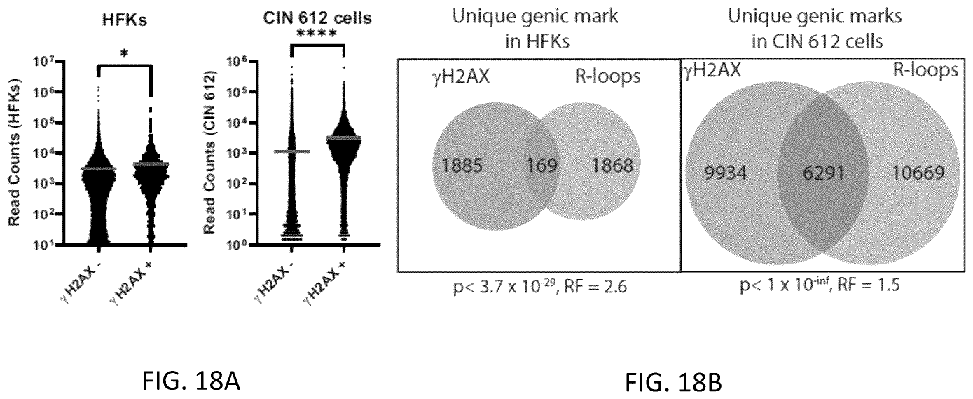

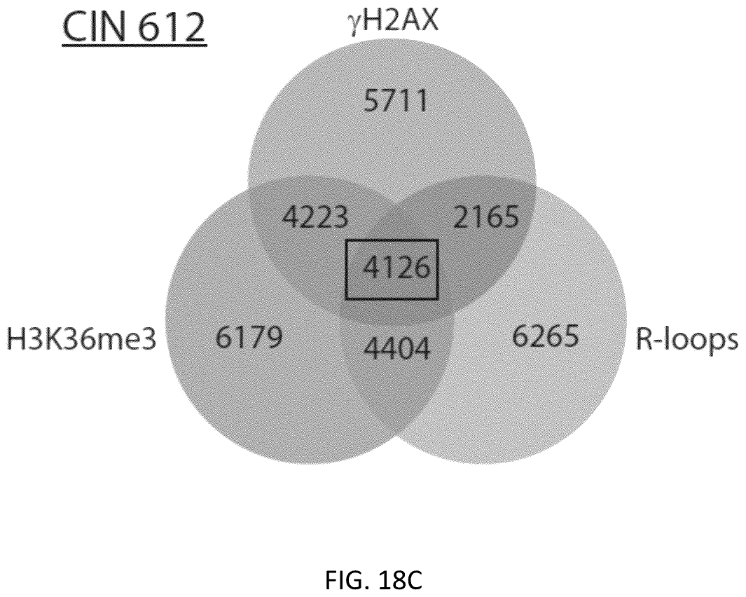

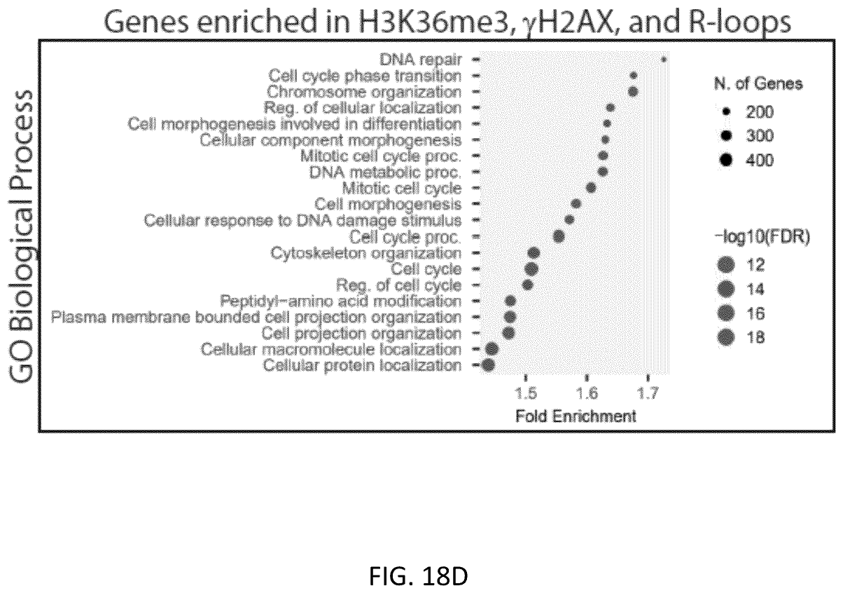

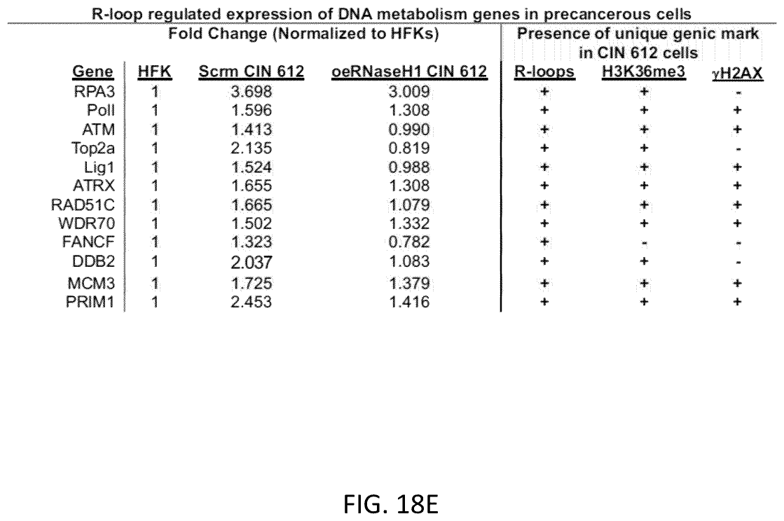

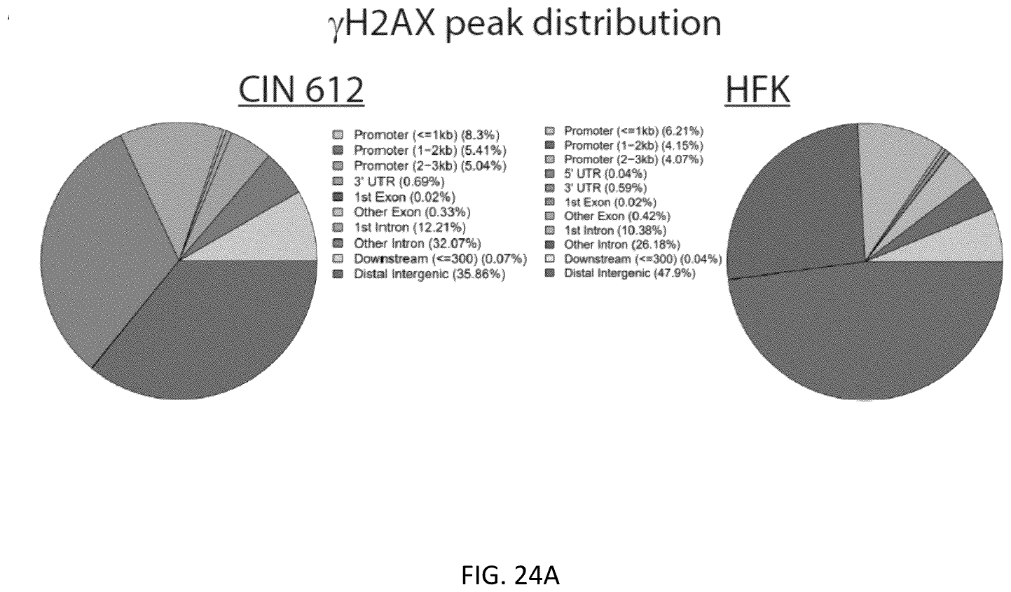

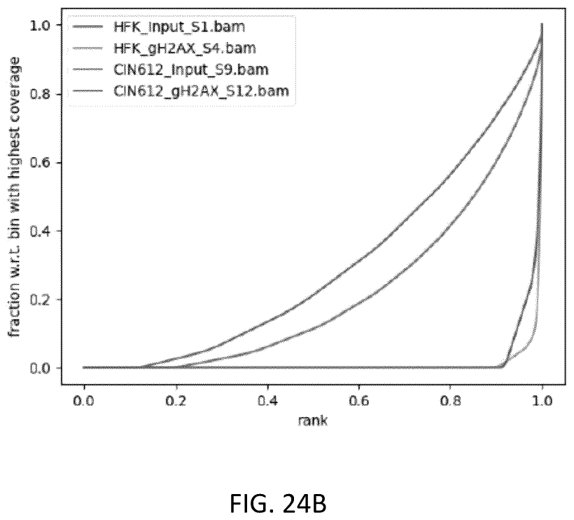

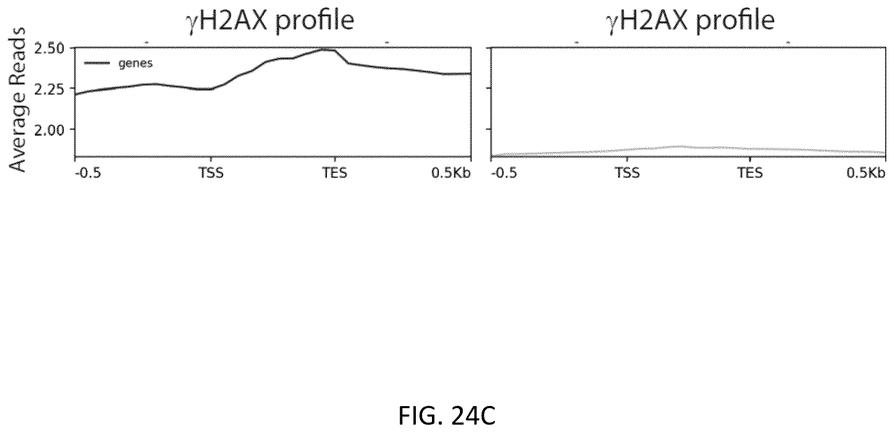

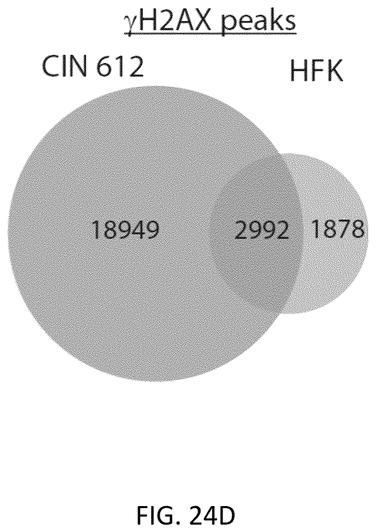

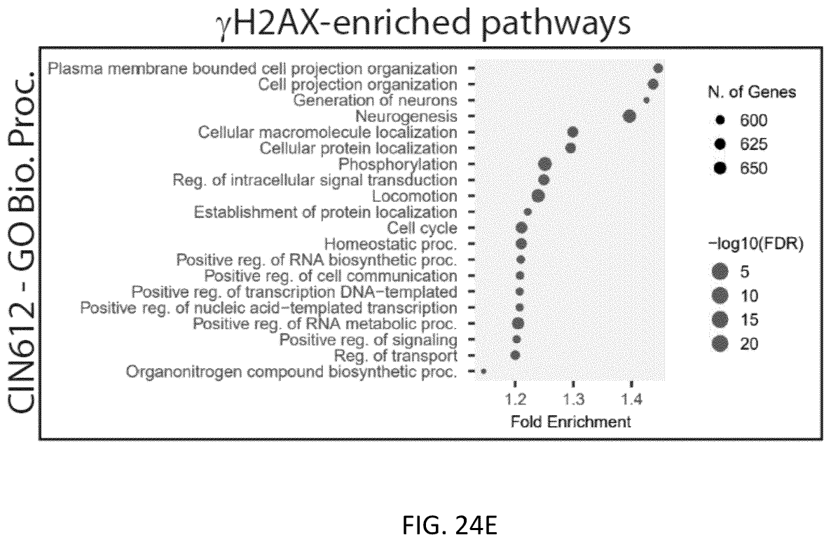

HPV positive cells contain high levels of modified H2AX (γH2AX) and DNA breaks, which results in the constitutive activation of DNA damage repair pathways [28, 51]. Consistent with these findings, peak calling analysis identified ˜4-fold more 7H2AX marks (21,941 vs. 4,870) in the CIN 612 cells than in the normal cells (FIG. 24D). In addition, the profile of averaged γH2AX reads across genic regions of precancerous CIN612 cells differed substantially from that seen in the normal keratinocytes. While normal keratinocytes exhibited no significant increases in γH2AX reads across genic regions, γH2AX reads in HPV positive cells increased, peaking at the TES (FIG. 24C). Pathway enrichment analysis of genes containing γH2AX marks in the precancerous CIN 612 cells identified those responsible for cell cycle control, regulation of RNA biosynthetic processes, and transcription. This enrichment was only seen in HPV positive cells and not in normal keratinocytes. Interestingly, γH2AX was associated with genes with ˜2-fold higher transcript levels than those without γH2AX (FIG. 18A). Approximately 37% of all genic R-loops in CIN 612 cells were found to be associated with γH2AX in contrast to normal keratinocytes, where negligible levels were detected (FIG. 18B). It was next important to determine whether H3K36me3 or H3K9me3 were preferentially associated with γH2AX and R-loops. Interestingly, about 25% of all R-loop-containing genes in CIN 612 cells were found to be positive for the combination of R-loops, H3K36me3, and γH2AX (FIG. 18C). This level of correlation was not seen in normal cells nor with H3K9me3 and γH2AX. The R-loop associated genes which were H3K36me3 and γH2AX positive, are involved in pathways essential for viral replication, including DNA break repair and cell cycle control (FIG. 18D).

The association of H3K36me3, γH2AX, and enhanced R-loops was particularly significant for genes in the DNA repair pathway. HPV proteins activate the ATM and ATR DNA repair pathways, which is critical for differentiation-dependent amplification. Our studies show that genes like ATM, ATRX, RAD51C, along with members of the Fanconi Anemia pathway (FANC-B, C, E, I, L, and M), and SETD2, the methyltransferase regulating H3K36me3, were all associated with the combination of H3K36me3, γH2AX and enhanced R-loops (FIG. 18E). While a significant linkage was found between innate immune regulatory genes and the presence of both R-loops and H3K36me3, only a minimal association was found for the combination of H3K36me3, γH2AX, and enhanced R-loops. This suggests there may be a preferred linkage of H3K36me3, γH2AX, and enhanced R-loops with genes in the DNA damage repair pathway. These results indicate that the linkage between all three factors, γH2AX, H3K36me3, and R-loops, is critical for HPV pathogenesis and cancer progression.

### DISCUSSION

The levels of R-loops are increased in many cancers, and how the distribution, as well as the function of these structures, change due to the presence of high-risk HPV genomes was examined by comparing cells derived from an HPV 31 positive precancerous lesion of the cervix (CIN I) to normal keratinocytes. The levels of R-loops were found to be enriched by ˜5-10 fold on individual cellular genes in CIN 612 cells in comparison to normal keratinocytes. The largest enrichment of R-loops identified in HPV positive cells was, however, associated with repetitive ALU elements, which exhibited over a 500-fold increase compared to that seen in normal keratinocytes. While the levels of R-loops are significantly increased in HPV positive cells, the overall pattern of where R-loops form on cellular genes is very similar to that detected in normal keratinocytes, with peak levels located within 2 kb upstream of start sites, within gene bodies, as well as 2 kb downstream of termination sequences. Approximately one-third of the R-loops identified in CIN 612 cells are located at sites similar to those found in normal keratinocytes, while about two-thirds of the R-loops are associated with unique genes only in the HPV positive cells and not in normal keratinocytes. Interestingly, the expression of genes with R-loops associated only in CIN 612 cells is divided equally between those with increased or decreased transcript levels. While no global increase in expression is associated with enhanced R-loop levels, genes in specific pathways were found to be coordinately regulated. This includes pathways associated with DNA repair, DNA replication and cell cycle, whose expression is coordinately increased. Equally interesting is the identification of genes involved in innate immune surveillance and keratinocyte differentiation, which are suppressed. All these changes may contribute to progression from normal to precancerous states as well as for the pathogenesis of high-risk HPVs, which are the etiological agents responsible for cervical intraepithelial neoplasia. This indicates that the directed formation of R-loops on specific groups of genes may provide an important function in the HPV life cycle.

The repression of genes in the innate immune surveillance pathway in the CIN 612 cells is particularly sensitive to enhanced levels of R-loops. In wild type CIN 612 cells, the expression of many innate immune regulatory genes is reduced by 2 to 5-fold from that seen in normal keratinocytes (FIG. 16B). The stable overexpression of RNase H1 in CIN 612 cells resulted in increased expression of innate immune genes, including STAT1 (8-fold), NLRP3 (14-fold), AIM2 (34-fold), IL6 (190-fold), and IFN β (over 50-fold), indicating their repression may be functionally linked to R-loop levels. Importantly, R-loops are only found to be associated with these cellular genes in the CIN 612 cells. Some of these increases exceed the amounts seen in normal keratinocytes, suggesting that multiple upstream regulators of these factors also depend on R-loop formation. Furthermore, the expression of several genes, such as RIG-I and TRIM 25, are increased by RNase H1 overexpression despite not being linked to the presence of R-loops. These are interferon stimulated genes, and the increases in expression are likely the result of the enhanced levels of IFN β that are induced when R-loop levels are reduced [52-57]. Along with increased expression of innate immune genes, RNase H1 overexpression also reduces viral gene expression and episome levels by ˜₇₀% and 50%, respectively. Whether these reductions are due to increased expression of innate immune regulators or a direct effect due to loss of R-loops on viral episomes is unclear. In contrast to the repression of the innate immune regulatory pathway, genes in the DNA damage repair pathway are activated by enhanced levels of R-loops in CIN 612 cells. This includes genes such as ATM, Top2A, Lig1, and RAD51C. While the differences seen with the DNA damage repair genes are not as dramatic as seen with the innate immune regulated genes, the activation of the DNA damage repair pathway in HPV positive cells has been shown to be critical for viral pathogenesis and cancer progression.

Our observation that R-loops are found in association with specific sets of genes that are linked with both increased and decreased expression indicates that their formation is not merely an accidental byproduct of increased transcription but is instead the result of a directed process. One way that expression could be linked with enhanced levels of R-loops is through altered chromatin states associated with specific sets of modified histones. Previous studies have suggested an association of H3K36me3 and H3K9me3 with certain classes of R-loops, but only a limited correlation with altered expression has been described [2, 58]. Our studies demonstrated that over half of R-loop associated genes in CIN I derived cells are associated with H3K36me3 marks, while only 8% are positive in normal keratinocytes. H3K36me3 has been linked with increased transcription; however, in our study, equal numbers of dually H3K36me3 and R-loop positive genes exhibit increased expression as decreased expression compared to normal keratinocytes [59-62]. This indicates that this histone mark is more likely associated with an accessible chromatin configuration rather than increased transcription alone. Both innate immune regulatory genes, as well as those in DNA damage repair, are linked with high levels of H3K36me3, and this is not seen in normal keratinocytes, demonstrating that this effect is specific to HPV positive cells. SETD2 is the methyltransferase that regulates the deposition of methyl groups on lysine 36 of histone 3 (H3K36me3), and its levels are increased in CIN 612 cells as well as other HPV positive cells [63-65]. Knockdown of SETD2 in HPV positive cells has been shown to lead to significant reductions in viral episomes, identifying it as an important regulator of viral persistence. While H3K36me3 has been identified as a mark of transcription elongation, recent studies have also linked it with DNA repair suggesting a potential link with genomic instability and DNA breaks [66-68]. A previous study linked cells with high R-loop levels to concomitant decreases in H3K9me3 levels, consistent with our studies, as CIN 612 cells contained far fewer of these marks than normal keratinocytes [69]. In contrast, no strong linkage was found between H3K9me3 and R-loop regulated gene expression in CIN 612 cells. Only 2% of R-loop associated genes were also positive for H3K9me3 as compared to 10% in normal keratinocytes.

The failure to resolve R-loops leads to the formation of DNA breaks and genomic instability [70]. HPV positive precancers, as well as other cancers, exhibit high levels of DNA break formation as indicated by enhanced amounts of 7H2AX, which is often used as a surrogate marker [71]. In CIN 612 cells, high levels of γH2AX are associated with increased levels of R-loops at genes whose expression is altered. Over one third of the genes associated with R-loops in the HPV positive cells were also positive for 7H2AX. In addition, 67% of the genes positive for both γH2AX and R-loops were also linked to H3K36me3. No such associations are seen in normal keratinocytes. Approximately 700 of the genes that are differentially expressed in the CIN 612 cells are linked to the combined presence of 7H2AX, H3K36me3, and R-loops. Genes whose expression is positively regulated by R-loop formation and associated with both γH2AX and H3K36me3 include ATM, ATRX, ATR, Top2A, and RPA3. At the same time, genes negatively regulated by R-loops that are also H3K36me3 and γH2AX positive include JAK2 and TRIM 14. The association of γH2AX and R-loops with DNA damage repair genes may be important but the mechanism responsible is unclear. Recent studies have suggested that γH2AX might not only interact with sites of endogenous DNA breaks but also associate with DNA intermediates that form upon chromatin opening during transcription initiation [72, 73]. The increased expression of genes linked with the combination of γH2AX, R-loops, and H3K36me3 in HPV positive cells compared to normal keratinocytes supports this model.

These studies identify a potential linkage between R-loops, specific histone marks, and altered transcription. However, additional factors must act to determine how genes in specific pathways are targeted. One such possibility may be the association with other non-β DNA structures like G-quadruplexes and GC skew. The relationship between G-quadruplex formation and stability of R-loops has been noted in multiple reports, and may contribute to effects in HPV positive cells [11, 74, 75]. Similarly, a GC skew has been reported in a number of R-loops, and a preliminary screening indicates that some but not all R-loops associated with innate immune genes have this skew, identifying an important area for future studies. In addition to structural motifs in DNAs, we have shown that inhibition of p53 leads to increased levels of R-loops in HPV positive cells, cells and has been reported in other tumor cell lines that have mutated p53 [20, 69]. This indicates that factors downstream of p53 play important roles in regulating R-loop formation and that this occurs at specific sites on cellular genes. Transient inhibition of p53 in normal keratinocytes alone is, however, not sufficient to induce increased R-loop formation but our studies have shown the requirement of HPV E7 co-expression, which implicates inhibition of Rb proteins as a possible contributing factor. Additional factors that could be downstream mediators of the p53 effects on R-loop formation include members of the p21-DREAM complex, long non-coding RNAs, and APOBEC 3B proteins. In embryonic mouse stem cells, a subset of polycomb group genes was shown to be linked with R-loop formation, and overexpression of RNase H1 increased their expression, indicating a repressive effect of R-loops [76]. At the same time, RNase H1 overexpression led to decreased expression of other polycomb genes and this differential regulation is similar to our results. This R-loop dependent activity requires the cooperative action of cellular proteins, and we believe that additional factors, including modified chromatin as well as transcription factors, can provide comparable functions in HPV positive cells. It is also possible that viral proteins can contribute to regulating the expression of R-loop associated genes. Overexpression of RNAse H1 in HPV-positive cells decreased the expression of viral early genes [20], and this reduction in viral proteins could potentially impact the expression of cellular genes that are linked to the presence of R-loops at these loci.

Overall, these observations demonstrate that R-loop levels are significantly elevated within HPV positive cells compared to normal keratinocytes. While no global effect on gene expression is seen due to increased levels of R-loops, genes in pathways that are important for viral replication and cellular transformation are coordinately activated or repressed by these structures, possibly in cooperation with the recruitment of specific types of modified histones. Our studies indicate that in HPV-positive cells, R-loops contribute to regulating cellular and viral gene expression during HPV pathogenesis, including those involved in the innate immune response and DNA damage repair.

### Materials and Methods

### Reagents

Antibodies used in these experiments were as follows: S9.6 (Millipore), Anti-Histone H3 (tri methyl K36) antibody—ChIP Grade (Abeam), Anti-Histone H3 (tri methyl K9) antibody—ChIP Grade (Abeam), Phospho-Histone H2A.X (Ser139) (D7T2V) Mouse mAb (Cell Signaling), and Mouse IgG (Diagenode). Methlyene Blue Hydrate (Sigma) was used for staining nucleic acids in dot blot assays. RNase H (ThermoFisher) was used to remove R-loops from nucleic acid extracts to determine specificity of the S9.6 antibody. Mung Bean Nuclease was purchased from New England Biologicals and was used for enzymatic digestion of samples during chromatin immunoprecipitation- and DNA:RNA immunoprecipitation-sequencing.

TABLE 4
DNA: RNA immunoprecipitation-qPCR primers

| | Forward Primer | Reverse Primer |
|---|---|---|
| Cellular regions | | |
| MYADM | 5′ CGT AGG TGC CCT AGT | 5′ TCC ATT CTC ATT CCC |
| | TGG GAG 3′ (SEQ ID NO: 25) | AAA CC 3′ (SEQ ID NO: 26) |
| RPL13a | 5′ AAT GTG GCA TTT CCT | 5′ CCA ATT CGG CCA AGA |
| | TCT CG 3′ (SEQ ID NO: 27) | CTC TA 3′ (SEQ ID NO: 28) |
| EGR1 | 5′ GAA CGT TCA GCC TCG | 5′ GGA AGG TGG AAG GAA |
| | TTC TC 3′ (SEQ ID NO: 29) | ACA CA 3′ (SEQ ID NO: 30) |
| SLC35B2 | 5′ AAG TCT TGC CCT AGC | 5′ GCC TAC ACC GCT TGT |
| | TGT GCT 3′ (SEQ ID NO: | GCT TTT 3′ (SEQ ID NO: 32) |
| | 31) | |
| SNRPN | 5′ GCC AAA TGA GTG AGG | 5′ TCC TCT CTG CCT GAC |
| | ATG GT 3′ (SEQ ID NO: 33) | TCC AT 3′ (SEQ ID NO: 34) |
| LGALS2 | 5′ TGA CCT CAC CTT GAC | 5′ AGC TGA ACC TGC ATT |
| | CTC TGA 3′ (SEQ ID NO: | TCA ACC 3′ (SEQ ID NO: 36) |
| | 35) | |
| ALU elements | 5′ ACG AGG TCA GGA GAT | 5′ CTC AGC CTC CCA AGT |
| | CGA GA 3′ (SEQ ID NO: 37) | AGC TG 3′ (SEQ ID NO: 38) |
| HPV 31 genomic | | |
| regions | | |
| Early PolyA | 5′ GGT ATT GGT ATT GGT | 5′ ACC CAT ACT ACC ATA |
| | ATT GG 3′ (SEQ ID NO: 39) | CCT TA 3′ (SEQ ID NO: 40) |
| Late PolyA | 5′ GCG TGT GTA CTT GTA | 5′ GCA ACC GAA AAC GGT |
| | 3′ (SEQ ID NO: 41) | TAG G 3′ (SEQ ID NO: 42) |
| Upstream regulatory | 5′ GAT GCA GTA GTT CTG | 5′ TAT GTT GGC AAG GTG |
| regions (URR) | CGG TTT 3′ (SEQ ID NO: | TGT TAG G 3′ (SEQ ID NO: |
| | 43) | 44) |

### Cell Culture and Reagents

### Isolation of HFKs

Neonatal human epidermis was supplied by the Skin Disease and Research Core at Northwestern University. These de-identified tissues were suspended in Hanks' balanced salt solution (HBSS), and isolations were performed within 3 to 4 days of circumcision. The foreskins were washed in phosphate-buffered saline (PBS) before being processed. Excess blood vessels, tissue, and fat were cleaned away before being incubated overnight at 4 C in 2.4 U/ml Dispase. The following day, the epidermis was removed and incubated with 4 ml of 0.25% trypsin for 15 min. The epidermis was then scraped vigorously for 2 to 3 min before quenching the trypsin with bovine serum. The resulting suspension was then pipetted through a 40 mm pore cell sieve. The cells were then spun down and resuspended in E-medium supplemented with 5 ng/ml of mouse epidermal growth factor (EGF). NIH 3T3-J2 fibroblasts, growth-arrested through treatment with mitomycin-c, were seeded with the newly collected human foreskin keratinocytes (HFKs), and media was changed as required until the proliferation of the keratinocytes was achieved.

### Cell Culture

HFKs, HFK-31, and CIN 612 cells were all cultured in E-medium supplemented with 5 ng/ml of mouse EGF. Each of these cell lines were co-cultured with NIH 3T3-J2 fibroblasts, which were growth arrested using 0.4 mg/ml of mitomycin-c for at least 2 hr. To remove J2 fibroblasts prior to downstream analyses, cells were washed with Versene (0.05 mM EDTA PBS) for 5 min before 2 sequential PBS washes. J2 feeders were cultured in DMEM containing 1% penicillin-streptomycin and 10% bovine serum. Cells stably overexpressing RNase H1 were generated previously [20].

### Generation of Cells Stably Maintaining HPV 31 Episomes

The pBR-322 min-HPV31 plasmid was digested such that the pBR-322 backbone was removed, leaving the HPV 31 genome which was recircularized. 1 μg of recircularized HPV 31 DNA was contransfected with a selection plasmid expressing a neomycin resistance cassette (PSV2neo) into around one million freshly isolated HFKs at ˜60% confluence. The following day, cells were selected using 200 mg/ml G418. J2 feeders were changed on alternating days as the G418 selection. Stable maintenance of HPV 31 episomal DNA was assessed by Southern blot before expanding and performing downstream analyses on these cells.

### S9.6 Dot Blot Assay

DNA was purified from cell lysates using PhenolChloroform extractions. Samples were either left untreated or treated with 1 U of RNase H for at least 1.5 hr at 37° C. DNA was then spotted onto a positively charged membrane (Zeta-probe). Membranes were then stained with Methylene blue for −15 min before being washed with di-H2O 3 times for 5 min. Images of the Methylene blue staining were acquired to normalize to total nucleic acid content using an Odyssey Fc LiCor (LiCor BioSciences). Methylene blue staining was removed through washing with 100% ethanol for 5 min before washing with di-H2O 3 times for 10 min. Membranes were then blocked with 5% Bovine Serum Albumin (BSA) in TBST (Tris-buffered saline Tween 20) before being probed with the S9.6 anti-RNA:DNA hybrid antibody (Millipore) overnight at 4° C. The following day, membranes were washed with TBST, probed with secondary antibody for 1 h at RT, and developed using enhanced chemiluminescence (ECL) (Fisher, 4500085). Images were taken using an Odyssey Fc LiCor.

DNA:RNA Immunoprecipitation (DRIP)—qPCR

1×10⁷ cells were harvested and collected in Southern lysis buffer before being treated with RNase A (5 ng/mL) and Proteinase K (7.5 ng/mL) at 37° C. overnight. DNA was purified from these samples using phenol-chloroform extractions, and 25 to 50 mg of DNA was used for each sample. DNA was sheared using a Bioruptor (Diagenode) on high power, 30 s on/90 s off cycles for 20 min or digested using 1 U of mung bean nuclease for 1 h at 37° C. Input DNA was removed before loading the samples into preblocked magnetic beads in IP buffer containing 2 mg of the RNA:DNA hybrid antibody. Immunoprecipitations were allowed to incubate overnight at 4° C. while rotating. The next day, samples were washed 8 times with RIPA buffer for 5 min while rotating. One wash in TE buffer was performed before samples were eluted for 10 min at 65° C. in 10% sodium dodecyl sulfate (SDS), 10 mM Tris pH 7.4, 50 mM ethylenediaminetetraacetic acid (EDTA). DNA was purified from these elutions using a PCR purification kit (Qiagen) and stored at −20° C. Primer sets used to analyze S9.6 immunoprecipitated sequences are listed in the Key Resources table (Table 4).

### DRIP-Sequencing

The same protocol was used to prepare samples for DRIP-sequencing as listed above for DRIP-qPCR. Samples were stored at −80° C. until being shipped to Admera Biosciences (NJ), who performed the sequencing experiments. Briefly, the library was prepared using a KAPA HyperPrep Kit (Kapa Biosystems) following the manufacturer's recommendation. Input DNA was end-repaired and 3′-dA tailed. Adapter was then ligated to the DNA, and the ligated product was PCR amplified and cleaned up using the SPRIselect Reagent (Beckman Coulter). Quality control was then performed for the final library, followed by sequencing.

### DRIP-Sequencing Data Analysis

Admera Biosciences (NJ) performed most of the bioinformatic analyses from our DRIP-sequencing experiments. Their bioinformatics methods are as follows: An in-house bioinformatics pipeline was used to analyze DRIP-Seq data. First, FastQC (v0.11.8) was used to check the quality of raw and trimmed reads. Trimmomatic (v0.38) was used to cut adapters and trim low-quality bases with a default setting. BWA (v0.7.10-r789) was used to map the trimmed reads to the reference genome* using the Burrows-Wheeler Alignment algorithm (BWA-MEM). Mapped reads that have low-quality MAPQ score (MAPQ<10), not-properly-paired, or duplicated (assessed with Picard tools (v 2.20.4)) were removed. BAM was used to generate BW format (normalized by RPKM) for visualization. MACS (v2.2.4) was chosen to call peaks. If there was no replicate, the R package MAnorm (v2.2.6) was used for sample comparison. On the other hand, if there were replicates, their called peaks were merged and the DiffBind package (v2.14.0) was then used for differential analysis. Peak annotation and combined density profiles were performed by the ChIPseeker package (v1.22.1) and deepTools, respectively.

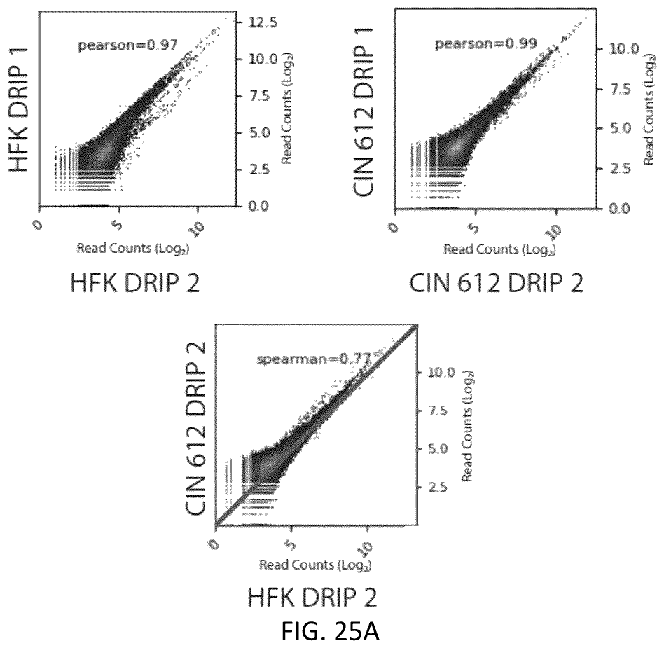

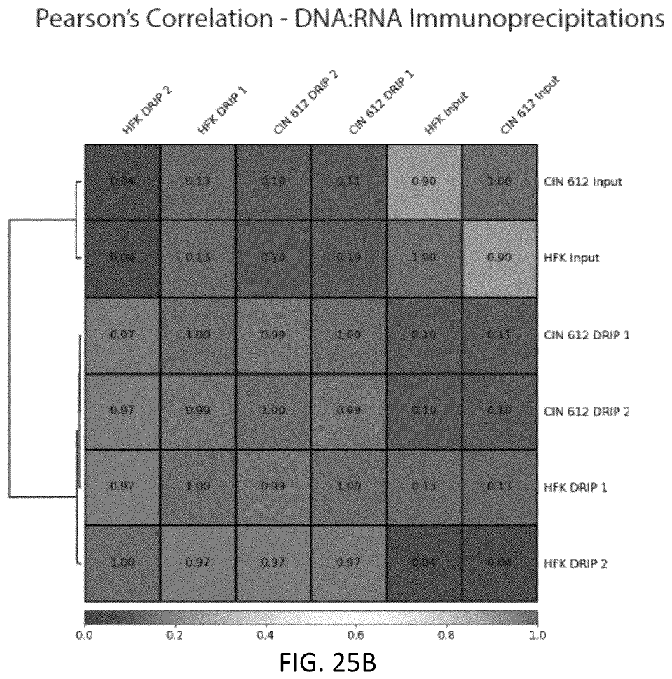

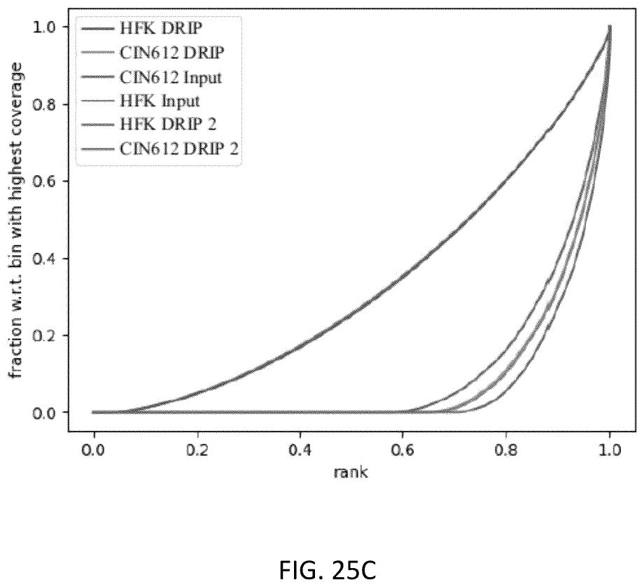

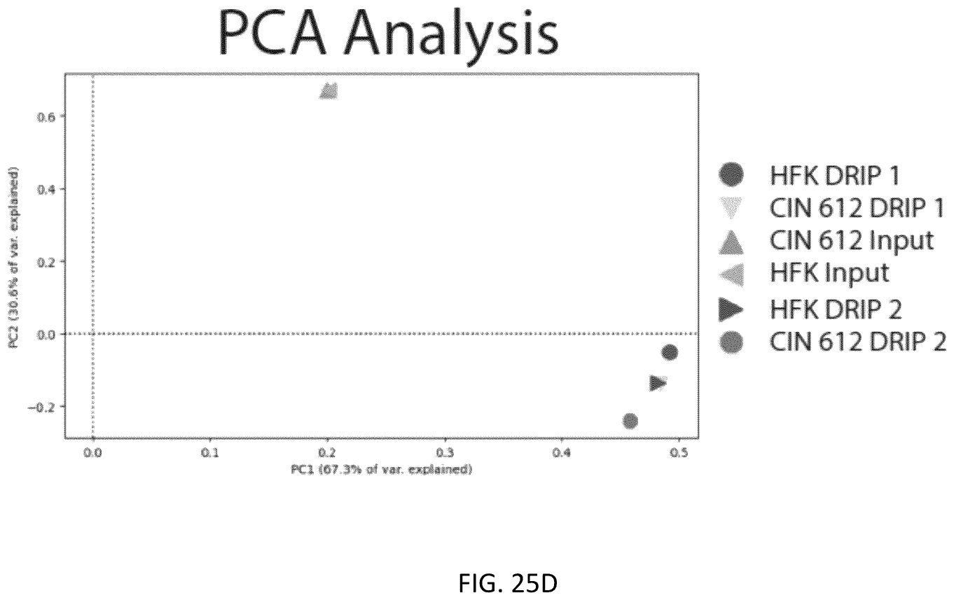

We performed the profile analysis of multiple DRIP-seq replicates from HFKs and CIN 612 cells (FIG. 13D) and R-loop read distribution (FIG. 14C) using the open-source Galaxy servers https://usegalaxy.org/. BAM Compare (Galaxy Version 3.5.4+galaxy0) was used to normalize either log 2 IP to input ratios, or input subtracted IP reads for both biological replicates of DRIP-seq in HFK and CIN 612 cells. ComputeMatrix was used to prepare files for visualization via plotProfile and plotHeatmap (FIG. 13D). CHIPseeker (Galaxy Version 1.28.3+galaxy0) was used on the BED files generated by Admera Biosciences to determine the genomic distribution of R-loop peaks. To confirm agreement between our biological replicates (FIGS. 25A-D), correlations between replicates were assessed using multiBAMSummary (Galaxy Version 3.5.4+galaxy0), and then plotting principal component analyses using plotPCA (Galaxy Version 3.5.4+galaxy0) and plotting Pearson coefficients as a heatmap using plotCorrelation (Galaxy Version 3.5.4+galaxy0). plotFingerprint (Galaxy Version 3.5.4+galaxy0) was used to determine narrow versus broad distributions of S9.6 reads across the genome.

### RNA-Sequencing

HFKs and CIN 612 cells were grown to confluency on 10 cm dishes before removing J2 fibroblasts. Cells were scraped and centrifuged before being stored at −80° C. before shipping to Admera Biosciences (NJ).

### RNA-Sequencing Data Analysis

FastQC (version v0.11.8) was applied to check the quality of raw reads. Trimmomatic (version v0.38) was applied to cut adaptors and trim low-quality bases with default setting. STAR Aligner version 2.7.1a was used to align the reads. Picard tools (version 2.20.4) was applied to mark duplicates of mapping. The StringTie version 2.0.4 was used to assemble the RNA-Seq alignments into potential transcripts. The featureCounts (version 1.6.0)/HTSeq was used to count mapped reads for genomic features such as genes, exons, promoter, gene bodies, genomic bins and chromosomal locations. The De-Seq2 (version 1.14.1) was used to do the differential analysis. Pathway analyses were performed using Shiny GO http://bioinformatics.sdstate.edu/go/.

### Chromatin Immunoprecipitation (ChIP)-Sequencing

Formaldehyde was added to 1×10⁷ cells to a final concentration of 1% for 10 min at room temperature. Excess formaldehyde was quenched upon adding 0.125M glycine before then washing samples with PBS. Cells were then incubated in collection buffer (0.1M TrisHCl pH 9.4 and 10 mM DTT containing Roche Protease Inhibitor Cocktail) for 10 min on ice. Cells were then collected and spun down before being sequentially washed and incubated with NCP1 (10 mM EDTA, 0.5 mM EGTA, 10 mM HEPES pH 6.5, 0.25% Triton X100) and NCP2 (1 mM EDTA, 0.5 mM EGTA, 10 mM HEPES, and 200 mM NaCl) before being lysed in 0.5% Empigen BB, 1% SDS, 10 mM EDTA, 50 mM Tris HCl pH 8.0 containing Roche Protease Inhibitor Cocktail for 30 min on ice. Samples were then sonicated using a Bioruptor (Diagenode) on high power, 30 s on/90 s off cycles for 20 min. After sonication, samples were prepared exactly as described above in the DRIP-qPCR protocol. Samples were stored at −80° C. before being sent off for sequencing either by Admera Biosciences (NJ) or the NUseq facility at Northwestern University.

### ChIP-Sequencing Data Analysis

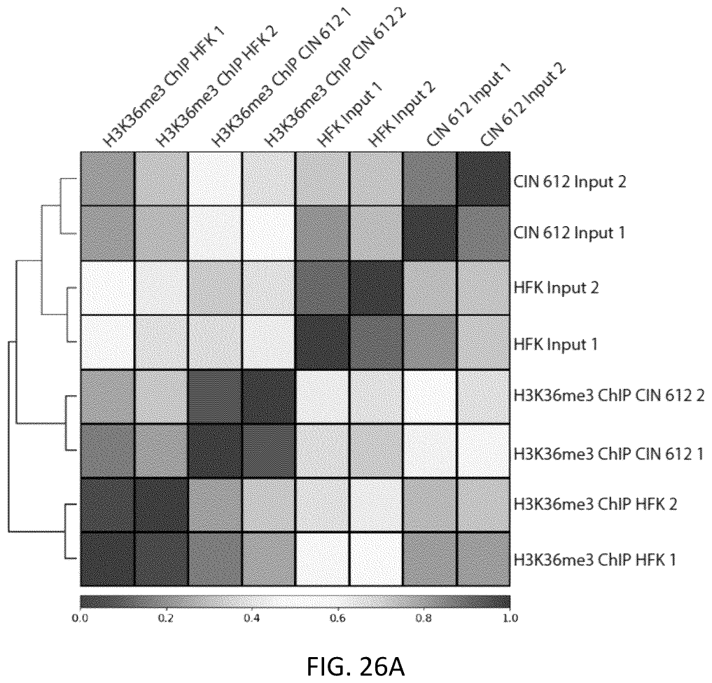

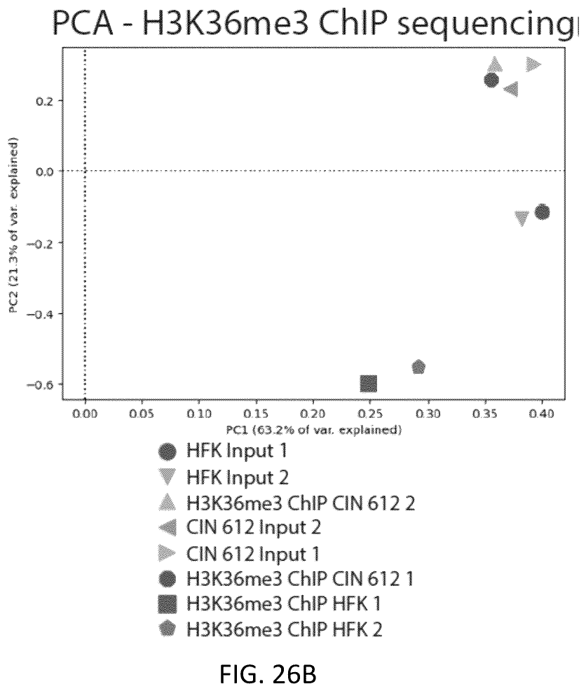

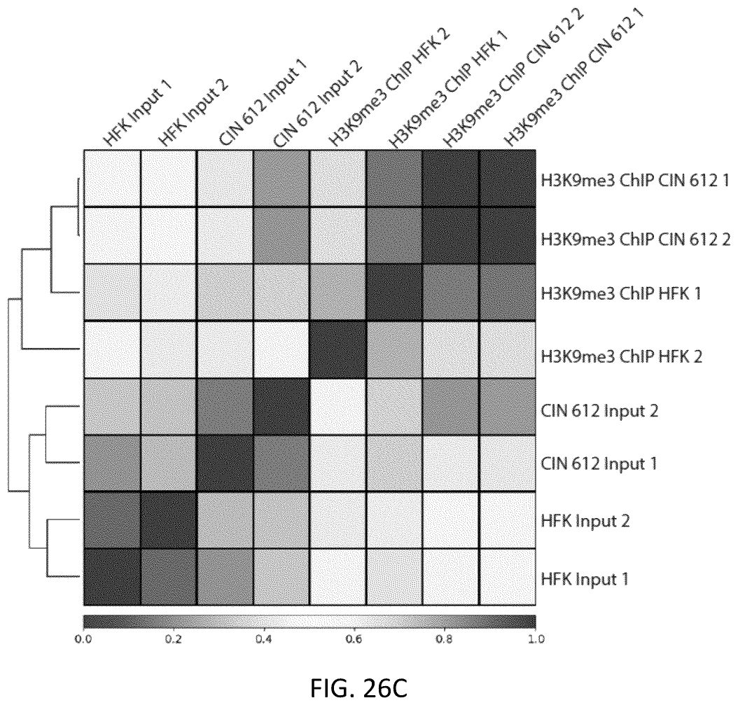

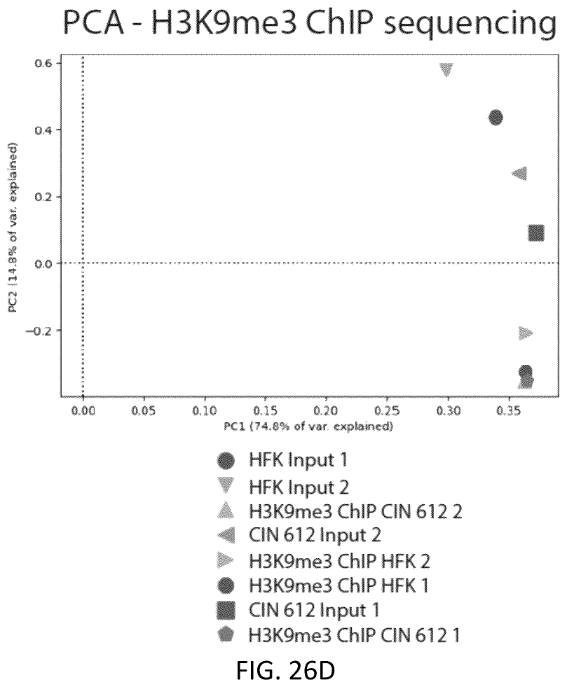

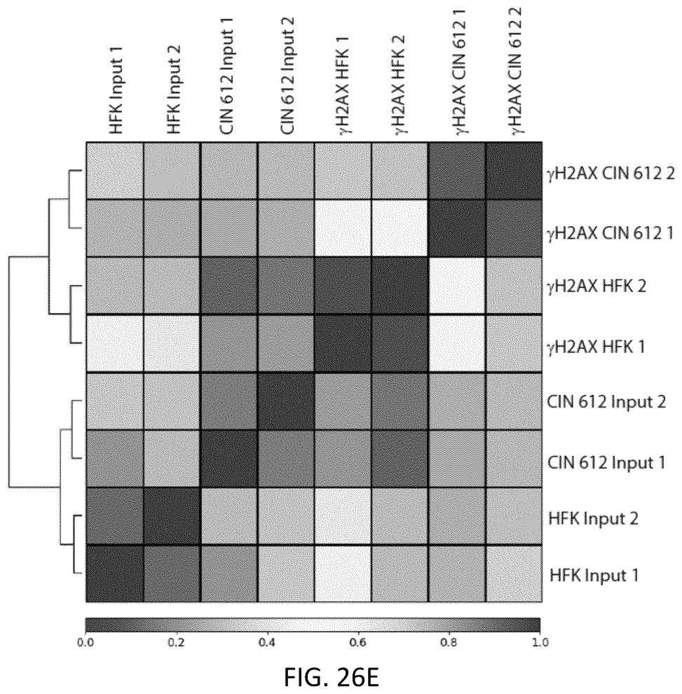

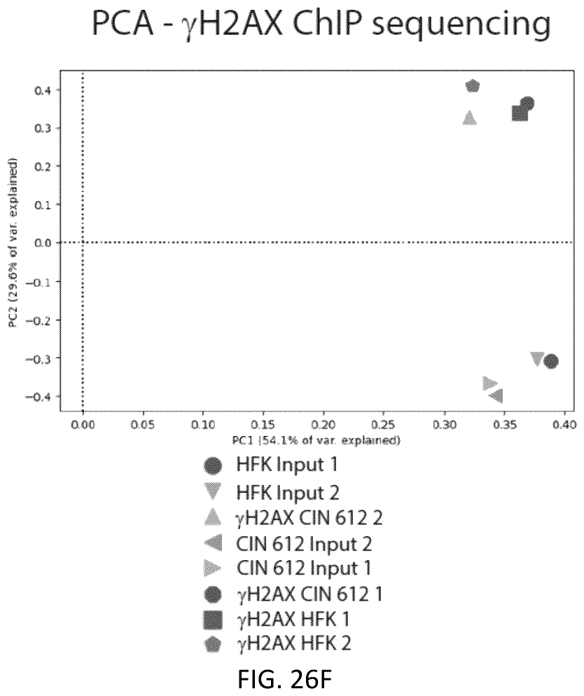

Samples were either analyzed as described above in the DRIP-sequencing analysis section or the NU seq core delivered BAM files. Agreement between biological replicates was assessed using multiBAMSummary (Galaxy Version 3.5.4+galaxy0), and then plotting principal component analyses using plotPCA (Galaxy Version 3.5.4+galaxy0) and plotting Pearson coefficients as a heatmap using plotCorrelation (Galaxy Version 3.5.4+galaxy0) (FIGS. 26A-F). From the BAM files, BAM Compare (Galaxy Version 3.5.4+galaxy0) was used to normalize log₂ IP to input ratios for both biological replicates of H3K36me3, H3K9me3, and γH2AX ChIPs from HFK and CIN 612 cells. ComputeMatrix was used to prepare files for visualization via plotProfile and plotHeatmap. CHIPseeker (Galaxy Version 1.28.3+galaxy0) was used on the BED files generated by Admera Biosciences or NUseq to determine the genomic distribution of each modified histone. MACS (v2.2.4) was used to call peaks. HOMER (Galaxy Version 4.11+galaxy0) was used to annotate where peaks occurred relative to their genomic location (intron, exon, etc.) and the corresponding gene name. Gene lists from HOMER were compared between DRIP, H3K36me3 ChIP, H3K9me3 ChIP, and γH2AX ChIP to obtain the overlap depicted in the Venn Diagrams. RNA counts from RNA sequencing experiments were compared to genes containing the corresponding mark (R-loops, H3K36me3, γH2AX or H3K9me3) to determine the association between mRNA levels seen in FIGS. 15D, 17A, and 18A.

### Quantification and Statistical Analysis

GraphPad prism was used for all statistical analyses, and all data are represented as mean+/−standard error (SEM). Two-way ANOVA and two-tailed T-tests were used to calculate p-values. Calculation of the representation factor and the associated probability of Venn diagram overlaps in FIGS. 17B and 18B were performed using http://nemates.org/MA/progs/overlap_stats.html from the Lund Lab. A genome size of 63,755 (CHESS database, http://ccb.jhu.edu/chess) was used to determine representation factors for the Venn Diagrams in FIGS. 17B and 18B. The maximum value represented as statistically significant was p=0.05. Additional details on quantifications like replicates are specifically stated in the figure legends and methods.

### Software and Algorithms

GraphPad Prism was used to generate all graphs and statistical analyses of said graphs. Adobe Photoshop and Illustrator were used for the organization and preparation of digital figures. Integrated Genome Browser (BioViz) generated depth graphs of S9.6 coverage in HFK and CIN 612 cells (FIGS. 13E-H). Galaxy community servers were used to perform many of the sequencing analyses [77].

### REFERENCES

1. Aguilera A, Garcia-Muse T. R Loops: From Transcription Byproducts to Threats to Genome Stability. Molecular cell. 2012; 46(2):115-24.
2. Sanz Lionel A, Hartono Stella R, Lim Yoong W, Steyaert S, Rajpurkar A, Ginno Paul A, et al. Prevalent, Dynamic, and Conserved R-Loop Structures Associate with Specific Epigenomic Signatures in Mammals. Molecular cell. 2016; 63(1):167-78.
3. Yu K, Chedin F, Hsieh C L, Wilson T E, Lieber M R. R-loops at immunoglobulin class switch regions in the chromosomes of stimulated B cells. Nat Immunol. 2003; 4(5):442-51.
4. Petermann E, Lan L, Zou L. Sources, resolution and physiological relevance of R-loops and RNA-DNA hybrids. Nature Reviews Molecular Cell Biology. 2022; 23(8):521-40.
5. Crossley M P, Bocek M, Cimprich K A. R-Loops as Cellular Regulators and Genomic Threats. Molecular cell. 2019; 73(3):398-411.
6. Allison D F, Wang G G. R-loops: formation, function, and relevance to cell stress. Cell stress. 2019; 3(2):38-46.
7. Chedin F, Benham C J. Emerging roles for R-loop structures in the management of topological stress. The Journal of biological chemistry. 2020; 295(14):4684-95.
8. Choo JAMY, Schlosser D, Manzini V, Magerhans A, Dobbelstein M. The integrated stress response induces R-loops and hinders replication fork progression. Cell Death & Disease. 2020; 11(7):538.
9. Edwards D S, Maganti R, Tanksley J P, Luo J, Park J J H, Balkanska-Sinclair E, et al. BRD4 Prevents R-Loop Formation and Transcription-Replication Conflicts by Ensuring Efficient Transcription Elongation. Cell Rep. 2020; 32(12):108166.
10. Lam F C, Kong Y W, Huang Q, Vu Han T-L, Maffa A D, Kasper E M, et al. BRD4 prevents the accumulation of R-loops and protects against transcription-replication collision events and DNA damage. Nature communications. 2020; 11(1):4083.
11. Lee C-Y, McNerney C, Ma K, Zhao W, Wang A, Myong S. R-loop induced G-quadruplex in non-template promotes transcription by successive R-loop formation. Nature communications. 2020; 11(1):3392.
12. Promonet A, Padioleau I, Liu Y, Sanz L, Biernacka A, Schmitz A-L, et al. Topoisomerase 1 prevents replication stress at R-loop-enriched transcription termination sites. Nature communications. 2020; 11(1):3940.
13. Yoon J, Hwang Y, Yun H, Chung J M, Kim S, Kim G, et al. LC3B drives transcription-associated homologous recombination via direct interaction with R-loops. Nucleic acids research. 2024.
14. Chakraborty P, Huang J T J, Hiom K. DHX9 helicase promotes R-loop formation in cells with impaired RNA splicing. Nature communications. 2018; 9(1):4346.
15. Jaiswal A S, Dutta A, Srinivasan G, Yuan Y, Zhou D, Shaheen M, et al. TATDN2 resolution of R-loops is required for survival of BRCA1-mutant cancer cells. Nucleic acids research. 2023; 51(22):12224-41.
16. Prendergast L, McClurg U L, Hristova R, Berlinguer-Palmini R, Greener S, Veitch K, et al. Resolution of R-loops by IN080 promotes DNA replication and maintains cancer cell proliferation and viability. Nature communications. 2020; 11(1):4534.
17. Stork C T, Bocek M, Crossley M P, Sollier J, Sanz L A, Chédin F, et al. Co-transcriptional R-loops are the main cause of estrogen-induced DNA damage. eLife. 2016; 5:e17548.
18. Kotsantis P, Silva L M, Irmscher S, Jones R M, Folkes L, Gromak N, et al. Increased global transcription activity as a mechanism of replication stress in cancer. Nature communications. 2016; 7(1):13087.
19. Tan S L W, Chadha S, Liu Y, Gabasova E, Perera D, Ahmed K, et al. A Class of Environmental and Endogenous Toxins Induces BRCA2 Haploinsufficiency and Genome Instability. Cell. 2017; 169(6):1105-18.e15.
20. Templeton C W, Laimins L A. p53-dependent R-loop formation and HPV pathogenesis. Proceedings of the National Academy of Sciences of the United States of America. 2023; 120(35):e2305907120.
21. de Martel C, Plummer M, Vignat J, Franceschi S. Worldwide burden of cancer attributable to HPV by site, country and HPV type. Int J Cancer. 2017; 141(4):664-70.
22. Kahn J A, Brown D R, Ding L, Widdice L E, Shew M L, Glynn S, et al. Vaccine-type human papillomavirus and evidence of herd protection after vaccine introduction. Pediatrics. 2012; 130(2):e249-56.
23. Vu M, Yu J, Awolude O A, Chuang L. Cervical cancer worldwide. Current problems in cancer. 2018; 42(5):457-65.
24. Castle P E, Murokora D, Perez C, Alvarez M, Quek S C, Campbell C. Treatment of cervical intraepithelial lesions. International Journal of Gynecology & Obstetrics. 2017; 138:20-5.
25. Bruno M T, Cassaro N, Bica F, Boemi S. Progression of CIN1/LSIL HPV Persistent of the Cervix: Actual Progression or CIN3 Coexistence. Infect Dis Obstet Gynecol. 2021; 2021:6627531.
26. Nedjai B, Reuter C, Ahmad A, Banwait R, Warman R, Carton J, et al. Molecular progression to cervical precancer, epigenetic switch or sequential model?Int J Cancer. 2018; 143(7):1720-30.
27. Moody C. Mechanisms by which HPV Induces a Replication Competent Environment in Differentiating Keratinocytes. Viruses. 2017; 9(9).
28. Moody C A, Laimins L A. Human Papillomaviruses Activate the ATM DNA Damage Pathway for Viral Genome Amplification upon Differentiation. PLOS Pathogens. 2009; 5(10):e1000605.
29. Pyeon D, Pearce S M, Lank S M, Ahlquist P, Lambert P F. Establishment of Human Papillomavirus Infection Requires Cell Cycle Progression. PLOS Pathogens. 2009; 5(2):e1000318.
30. De Geest K, Turyk M E, Hosken M I, Hudson J B, Laimins L A, Wilbanks G D. Growth and differentiation of human papillomavirus type 31b positive human cervical cell lines. Gynecol Oncol. 1993; 49(3):303-10.
31. Fehrmann F, Laimins L A. Human papillomavirus type 31 life cycle: methods for study using tissue culture models. Methods in molecular biology (Clifton, N J). 2005; 292:317-30.
32. Mehta K, Laimins L, Imperiale M J, Munger K, Androphy E. Human Papillomaviruses Preferentially Recruit DNA Repair Factors to Viral Genomes for Rapid Repair and Amplification. mBio. 2018; 9(1):e00064-18.
33. Crane H, Carr I, Hunter K D, E1-Khamisy S F. Senataxin modulates resistance to cisplatin through an R-loop mediated mechanism in HPV-associated Head and Neck Squamous Cell Carcinoma. bioRxiv. 2024:2024.02.22.581374.
34. Ozbun M A, Patterson N A. Using organotypic (raft) epithelial tissue cultures for the biosynthesis and isolation of infectious human papillomaviruses. Curr Protoc Microbiol. 2014; 34:14b.3.1-8.
35. Loomis E W, Sanz L A, Chédin F, Hagerman P J. Transcription-associated R-loop formation across the human FMR1 CGG-repeat region. PLoS Genet. 2014; 10(4):e1004294.
36. Jurga M, Abugable A A, Goldman A S H, E1-Khamisy S F. USP11 controls R-loops by regulating senataxin proteostasis. Nature communications. 2021; 12(1):5156.
37. Li L, Matsui M, Corey D R. Activating frataxin expression by repeat-targeted nucleic acids. Nature communications. 2016; 7(1):10606.
38. Ginno P A, Lott P L, Christensen H C, Korf I, Chédin F. R-loop formation is a distinctive characteristic of unmethylated human CpG island promoters. Molecular cell. 2012; 45(6):814-25.
39. Ge S X, Jung D, Yao R. ShinyGO: a graphical gene-set enrichment tool for animals and plants. Bioinformatics. 2019; 36(8):2628-9.
40. Bedard M C, Chihanga T, Carlile A, Jackson R, Brusadelli M G, Lee D, et al. Single cell transcriptomic analysis of HPV16-infected epithelium identifies a keratinocyte subpopulation implicated in cancer. Nature communications. 2023; 14(1):1975.
41. Bienkowska-Haba M, Luszczek W, Zwolinska K, Scott R S, Sapp M. Genome-Wide Transcriptome Analysis of Human Papillomavirus 16-Infected Primary Keratinocytes Reveals Subtle Perturbations Mostly due to E7 Protein Expression. J Virol. 2020; 94(3).
42. Bubeck D, Reijns M A M, Graham S C, Astell K R, Jones E Y, Jackson A P. PCNA directs type 2 RNase H activity on DNA replication and repair substrates. Nucleic acids research. 2011; 39(9):3652-66.
43. Cerritelli S M, Sakhuja K, Crouch R J. RNase H1, the Gold Standard for R-Loop Detection. Methods in molecular biology (Clifton, NJ). 2022; 2528:91-114.
44. Chédin F. Nascent Connections: R-Loops and Chromatin Patterning. Trends Genet. 2016; 32(12):828-38.
45. Jayakumar S, Patel M, Boulet F, Aziz H, Brooke G N, Tummala H, et al. PSIP1/LEDGF reduces R-loops at transcription sites to maintain genome integrity. Nature communications. 2024; 15(1):361.
46. Scalera C, Ticli G, Dutto I, Cazzalini O, Stivala L A, Prosperi E. Transcriptional Stress Induces Chromatin Relocation of the Nucleotide Excision Repair Factor XPG. International journal of molecular sciences. 2021; 22(12).
47. Becker J S, Nicetto D, Zaret K S. H3K9me3-dependent heterochromatin: barrier to cell fate changes. Trends in Genetics. 2016; 32(1):29-41.
48. Bulut-Karslioglu A, Inti A, Ramirez F, Barenboim M, Onishi-Seebacher M, Arand J, et al. Suv39h-dependent H3K9me3 marks intact retrotransposons and silences LINE elements in mouse embryonic stem cells. Molecular cell. 2014; 55(2):277-90.
49. Becker J S, McCarthy R L, Sidoli S, Donahue G, Kaeding K E, He Z, et al. Genomic and proteomic resolution of heterochromatin and its restriction of alternate fate genes. Molecular cell. 2017; 68(6):1023-37. e15.
50. Nicetto D, Zaret K S. Role of H3K9me3 heterochromatin in cell identity establishment and maintenance. Curr Opin Genet Dev. 2019; 55:1-10.
51. Gillespie K A, Mehta K P, Laimins L A, Moody C A. Human papillomaviruses recruit cellular DNA repair and homologous recombination factors to viral replication centers. J Virol. 2012; 86(17):9520-6.
52. Cui X-F, Imaizumi T, Yoshida H, Borden E C, Satoh K. Retinoic acid-inducible gene-I is induced by interferon-γ and regulates the expression of interferon-γ stimulated gene 15 in MCF-7 cells. Biochemistry and cell biology. 2004; 82(3):401-5.
53. Imaizumi T, Hatakeyama M, Yamashita K, Yoshida H, Ishikawa A, Taima K, et al. Interferon-7 induces retinoic acid-inducible gene-I in endothelial cells. Taylor & Francis; 2004. p. 169-73.
54. Imaizumi T, Yagihashi N, Hatakeyama M, Yamashita K, Ishikawa A, Taima K, et al. Expression of retinoic acid-inducible gene-I in vascular smooth muscle cells stimulated with interferon-gamma. Life Sci. 2004; 75(10):1171-80.
55. Imaizumi T, Yagihashi N, Hatakeyama M, Yamashita K, Ishikawa A, Taima K, et al. Upregulation of retinoic acid-inducible gene-I in T24 urinary bladder carcinoma cells stimulated with interferon-gamma. Tohoku J Exp Med. 2004; 203(4):313-8.
56. Martin-Vicente M, Medrano L M, Resino S, Garcia-Sastre A, Martinez I. TRIM25 in the Regulation of the Antiviral Innate Immunity. Front Immunol. 2017; 8:1187.
57. Yang C, Shu J, Miao Y, Liu X, Zheng T, Hou R, et al. TRIM25 negatively regulates IKKε-mediated interferon signaling in black carp. Fish & Shellfish Immunology. 2023; 142:109095.
58. Garcia-Pichardo D, Cañas J C, Garcia-Rubio M L, Gómez-González B, Rondón A G, Aguilera A. Histone Mutants Separate R Loop Formation from Genome Instability Induction. Molecular cell. 2017; 66(5):597-609.e5.
59. Huang C, Zhu B. Roles of H3K36-specific histone methyltransferases in transcription: antagonizing silencing and safeguarding transcription fidelity. Biophys Rep. 2018; 4(4):170-7.
60. Krogan N J, Kim M, Tong A, Golshani A, Cagney G, Canadien V, et al. Methylation of Histone H3 by Set2 in Saccharomyces cerevisiae Is Linked to Transcriptional Elongation by RNA Polymerase II. Molecular and Cellular Biology. 2003; 23(12):4207-18.
61. Li J, Moazed D, Gygi S P. Association of the Histone Methyltransferase Set2 with RNA Polymerase II Plays a Role in Transcription Elongation*. Journal of Biological Chemistry. 2002; 277(51):49383-8.
62. Neri F, Rapelli S, Krepelova A, Incarnato D, Parlato C, Basile G, et al. Intragenic DNA methylation prevents spurious transcription initiation. Nature. 2017; 543(7643):72-7.
63. Gautam D, Johnson B A, Mac M, Moody C A. SETD2-dependent H3K36me3 plays a critical role in epigenetic regulation of the HPV31 life cycle. PLoS Pathog. 2018; 14(10):e1007367.
64. Mac M, DeVico B M, Raspanti S M, Moody C A. The SETD2 Methyltransferase Supports Productive HPV31 Replication through the LEDGF/CtIP/Rad51 Pathway. J Virol. 2023; 97(5):e0020123.
65. Lam U T F, Tan B K Y, Poh J J X, Chen E S. Structural and functional specificity of H3K36 methylation. Epigenetics & Chromatin. 2022; 15(1):17.
66. Sun Z, Zhang Y, Jia J, Fang Y, Tang Y, Wu H, et al. H3K36me3, message from chromatin to DNA damage repair. Cell & Bioscience. 2020; 10(1):9.
67. Li F, Mao G, Tong D, Huang J, Gu L, Yang W, et al. The histone mark H3K36me3 regulates human DNA mismatch repair through its interaction with MutSα. Cell. 2013; 153(3):590-600.
68. Sharda A, Humphrey T C. The role of histone H3K36me3 writers, readers and erasers in maintaining genome stability. DNA Repair. 2022; 119:103407.
69. Panatta E, Butera A, Mammarella E, Pitolli C, Mauriello A, Leist M, et al. Metabolic regulation by p53 prevents R-loop-associated genomic instability. Cell Reports. 2022; 41(5):111568.
70. Marnef A, Legube G. R-loops as Janus-faced modulators of DNA repair. Nature Cell Biology. 2021; 23(4):305-13.
71. Alhmoud J F, Woolley J F, Al Moustafa A E, Malki M I. DNA Damage/Repair Management in Cancers. Cancers (Basel). 2020; 12(4).
72. Singh I, Ozturk N, Cordero J, Mehta A, Hasan D, Cosentino C, et al. High mobility group protein-mediated transcription requires DNA damage marker γ-H2AX. Cell Research. 2015; 25(7):837-50.
73. Dobersch S, Rubio K, Singh I, Gunther S, Graumann J, Cordero J, et al. Positioning of nucleosomes containing γ-H2AX precedes active DNA demethylation and transcription initiation. Nature communications. 2021; 12(1):1072.
74. Ribeiro de Almeida C, Dhir S, Dhir A, Moghaddam A E, Sattentau Q, Meinhart A, et al. RNA Helicase DDX1 Converts RNA G-Quadruplex Structures into R-Loops to Promote IgH Class Switch Recombination. Molecular cell. 2018; 70(4):650-62.e8.
75. Wulfridge P, Yan Q, Rell N, Doherty J, Jacobson S, Offley S, et al. G-quadruplexes associated with R-loops promote CTCF binding. Molecular cell. 2023; 83(17):3064-79.e5.
76. Skourti-Stathaki K, Torlai Triglia E, Warburton M, Voigt P, Bird A, Pombo A. R-Loops Enhance Polycomb Repression at a Subset of Developmental Regulator Genes. Molecular cell. 2019; 73(5):930-45.e4.

What is claimed is:

1. A method for detecting one or more human papillomaviruses (HPV) in a biological sample, comprising:
exposing a biological sample to an antibody or antibody fragment that binds to DNA-RNA hybrids;
detecting binding of the antibody or antibody fragment to DNA-RNA hybrids in the biological sample; and
determining that the biological sample contains HPV based on the detecting binding of the antibody or antibody fragment to DNA-RNA hybrids in the biological sample.

2. The method of claim 1, wherein the antibody or antibody fragment comprises a S9.6 antibody.

3. The method of claim 1, wherein the biological sample comprises a biopsy specimen.

4. The method of claim 3, wherein the biopsy specimen is a cervical biopsy specimen or an oropharyngeal biopsy specimen.

5. The method of claim 1, wherein the biological sample is from a subject having a tumor or suspected tumor, and wherein the biological sample comprises at least a portion of the tumor or suspected tumor.

6. The method of claim 1, wherein the determining comprises comparing a level of binding of the antibody or antibody fragment to DNA-RNA hybrids in the biological sample to a level of binding of the antibody or antibody fragment to DNA-RNA hybrids in a control sample.

7. The method of claim 6, wherein the control sample comprises tissue from the subject from a region not having a tumor or suspected tumor.

8. The method of claim 1, wherein the detecting binding of the antibody or antibody fragment to DNA-RNA hybrids in the biological sample comprises the use of an enzyme-linked immunosorbent assay (ELISA), immunohistochemistry, fluorescent microscopy, or a combination thereof.

9. The method of claim 5, further comprising therapeutically treating the subject, based on the determining that the biological sample contains HPV.

10. The method of claim 9, wherein the therapeutically treating the subject comprises removing the tumor or suspected tumor and/or administering one or more anti-cancer therapeutic agents.

11. A method for detecting one or more human papillomaviruses (HPV) in a biological sample, comprising:
exposing a first portion of the biological sample to an antibody or antibody fragment that binds to p16 protein;
detecting binding of the antibody or antibody fragment to p16 in the first portion of the biological sample;
exposing a second portion of the biological sample to an antibody or antibody fragment that binds to DNA-RNA hybrids; and
detecting binding of the antibody or antibody fragment to DNA-RNA hybrids in the second portion of the biological sample.

12. The method of claim 11, further comprising determining that the biological sample contains HPV based on: the detecting binding of the antibody or antibody fragment to p16 in the first portion of the biological sample; and the detecting binding of the antibody or antibody fragment to DNA-RNA hybrids in the second portion of the biological sample.

13. The method of claim 11, wherein the biological sample comprises a biopsy specimen.

14. The method of claim 13, wherein the biopsy specimen is a cervical biopsy specimen or an oropharyngeal biopsy specimen.

15. The method of claim 11, wherein the biological sample is from a subject having a tumor or suspected tumor, and wherein the biological sample comprises at least a portion of the tumor or suspected tumor.

16. The method of claim 11, wherein the antibody or antibody fragment that binds to DNA-RNA hybrids comprises a S9.6 antibody.

17. The method of claim 12, wherein the determining comprises comparing a level of binding of the antibody or antibody fragment to DNA-RNA hybrids in the second portion of the biological sample to a level of binding of the antibody or antibody fragment to DNA-RNA hybrids in a control sample.

18. The method of claim 17, wherein the control sample comprises tissue from the subject from a region not having a tumor or suspected tumor.

19. The method of claim 11, wherein the detecting binding of the antibody or antibody fragment to DNA-RNA hybrids in the second portion of the biological sample comprises the use of an enzyme-linked immunosorbent assay (ELISA), immunohistochemistry, fluorescent microscopy, or a combination thereof.

20. The method of claim 12, further comprising therapeutically treating the subject based on the determining that the biological sample contains HPV.

21. The method of claim 20, wherein therapeutically treating the subject comprises removing the tumor or suspected tumor and/or administering one or more anti-cancer therapeutics.


SEQUENCE LISTING (44 sequences)

SEQ ID NO: 1 - DNA, 21 bp, synthetic construct
  source 1..21: mol_type = other DNA
gccgtatgcaaagcacatgaa

SEQ ID NO: 2 - DNA, 21 bp, synthetic construct
  source 1..21: mol_type = other DNA
cctggtcattcgggatttata

SEQ ID NO: 3 - DNA, 21 bp, synthetic construct
  source 1..21: mol_type = other DNA
cactcaggatttgtgggcaat

SEQ ID NO: 4 - DNA, 21 bp, synthetic construct
  source 1..21: mol_type = other DNA
gcaaagccattgaacaagcaa

SEQ ID NO: 5 - DNA, 20 bp, synthetic construct
  source 1..20: mol_type = other DNA
acgaggtcaggagatcgaga

SEQ ID NO: 6 - DNA, 20 bp, synthetic construct
  source 1..20: mol_type = other DNA
ctcagcctcccaagtagctg

SEQ ID NO: 7 - DNA, 21 bp, synthetic construct
  source 1..21: mol_type = other DNA
gatgcagtagttctgcggttt

SEQ ID NO: 8 - DNA, 22 bp, synthetic construct
  source 1..22: mol_type = other DNA
tatgttggcaaggtgtgttagg

SEQ ID NO: 9 - DNA, 15 bp, synthetic construct
  source 1..15: mol_type = other DNA
gacctcggaaattgc

SEQ ID NO: 10 - DNA, 21 bp, synthetic construct
  source 1..21: mol_type = other DNA
aacatgctatgcaacgtcctg

SEQ ID NO: 11 - DNA, 21 bp, synthetic construct
  source 1..21: mol_type = other DNA
aattacccgacagctcagatg

SEQ ID NO: 12 - DNA, 20 bp, synthetic construct
  source 1..20: mol_type = other DNA
ggcacacgattccaaatcac

SEQ ID NO: 13 - DNA, 20 bp, synthetic construct
  source 1..20: mol_type = other DNA
tactgttgtggaagggcaag

SEQ ID NO: 14 - DNA, 22 bp, synthetic construct
  source 1..22: mol_type = other DNA
tcccagcaaaggatatttcgtc

SEQ ID NO: 15 - DNA, 16 bp, synthetic construct
  source 1..16: mol_type = other DNA
gacagacagacagggg

SEQ ID NO: 16 - DNA, 18 bp, synthetic construct
  source 1..18: mol_type = other DNA
cccgctgtctggaagttc

SEQ ID NO: 17 - DNA, 15 bp, synthetic construct
  source 1..15: mol_type = other DNA
gcgtgtgtacttgta

SEQ ID NO: 18 - DNA, 19 bp, synthetic construct
  source 1..19: mol_type = other DNA
gcaaccgaaaacggttagg

SEQ ID NO: 19 - DNA, 18 bp, synthetic construct
  source 1..18: mol_type = other DNA
gggagtgaccgaaactgg

SEQ ID NO: 20 - DNA, 17 bp, synthetic construct
  source 1..17: mol_type = other DNA
cgtgtggtgtgtcgtcc

SEQ ID NO: 21 - DNA, 20 bp, synthetic construct
  source 1..20: mol_type = other DNA
ggtattggtattggtattgg

SEQ ID NO: 22 - DNA, 20 bp, synthetic construct
  source 1..20: mol_type = other DNA
acccatactaccatacctta

SEQ ID NO: 23 - DNA, 21 bp, synthetic construct
  source 1..21: mol_type = other DNA
ggcttgtaacagctacccttc

SEQ ID NO: 24 - DNA, 23 bp, synthetic construct
  source 1..23: mol_type = other DNA
cttctggattctggcttataggg

SEQ ID NO: 25 - DNA, 21 bp, synthetic construct
  source 1..21: mol_type = other DNA
cgtaggtgccctagttgggag

SEQ ID NO: 26 - DNA, 20 bp, synthetic construct
  source 1..20: mol_type = other DNA
tccattctcattcccaaacc

SEQ ID NO: 27 - DNA, 20 bp, synthetic construct
  source 1..20: mol_type = other DNA
aatgtggcatttccttctcg

SEQ ID NO: 28 - DNA, 20 bp, synthetic construct
  source 1..20: mol_type = other DNA
ccaattcggccaagactcta

SEQ ID NO: 29 - DNA, 20 bp, synthetic construct
  source 1..20: mol_type = other DNA
gaacgttcagcctcgttctc

SEQ ID NO: 30 - DNA, 20 bp, synthetic construct
  source 1..20: mol_type = other DNA
ggaaggtggaaggaaacaca

SEQ ID NO: 31 - DNA, 21 bp, synthetic construct
  source 1..21: mol_type = other DNA
aagtcttgccctagctgtgct

SEQ ID NO: 32 - DNA, 21 bp, synthetic construct
  source 1..21: mol_type = other DNA
gcctacaccgcttgtgctttt

SEQ ID NO: 33 - DNA, 20 bp, synthetic construct
  source 1..20: mol_type = other DNA
gccaaatgagtgaggatggt

SEQ ID NO: 34 - DNA, 18 bp, synthetic construct
  source 1..18: mol_type = other DNA
tcctctctgcctgactcc

SEQ ID NO: 35 - DNA, 21 bp, synthetic construct
  source 1..21: mol_type = other DNA
tgacctcaccttgacctctga

SEQ ID NO: 36 - DNA, 21 bp, synthetic construct
  source 1..21: mol_type = other DNA
agctgaacctgcatttcaacc

SEQ ID NO: 37 - DNA, 20 bp, synthetic construct
  source 1..20: mol_type = other DNA
acgaggtcaggagatcgaga

SEQ ID NO: 38 - DNA, 20 bp, synthetic construct
  source 1..20: mol_type = other DNA
ctcagcctcccaagtagctg

SEQ ID NO: 39 - DNA, 20 bp, synthetic construct
  source 1..20: mol_type = other DNA
ggtattggtattggtattgg

SEQ ID NO: 40 - DNA, 20 bp, synthetic construct
  source 1..20: mol_type = other DNA
acccatactaccatacctta

SEQ ID NO: 41 - DNA, 15 bp, synthetic construct
  source 1..15: mol_type = other DNA
gcgtgtgtacttgta

SEQ ID NO: 42 - DNA, 19 bp, synthetic construct
  source 1..19: mol_type = other DNA
gcaaccgaaaacggttagg

SEQ ID NO: 43 - DNA, 21 bp, synthetic construct
  source 1..21: mol_type = other DNA
gatgcagtagttctgcggttt

SEQ ID NO: 44 - DNA, 22 bp, synthetic construct
  source 1..22: mol_type = other DNA
tatgttggcaaggtgtgttagg